(19) United States
(12) Patent Application Publication
(10) Pub. No.: US 2024/0148235 A1
(43) Pub. Date: May 9, 2024

(54) INFORMATION PROCESSING APPARATUS, INFORMATION PROCESSING METHOD, ENDOSCOPE SYSTEM, AND REPORT CREATION SUPPORT DEVICE

(71) Applicant: FUJIFILM Corporation, Tokyo (JP)

(72) Inventors: Yuma HORI, Kanagawa (JP); Yuya Kimura, Kanagawa (JP); Eiichi Imamichi, Kanagawa (JP)

(73) Assignee: FUJIFILM Corporation, Tokyo (JP)

(21) Appl. No.: 18/402,767

(22) Filed: Jan. 3, 2024

Related U.S. Application Data
(63) Continuation of application No. PCT/JP2022/025954, filed on Jun. 29, 2022.

(30) Foreign Application Priority Data
Jul. 7, 2021 (JP) 2021-113090
Dec. 3, 2021 (JP) 2021-196903

(51) Int. Cl. A61B 1/00; A61B 1/31; A61B 34/00; A61B 34/20; G06V 20/50; G16H 15/00; G16H 40/63

(52) U.S. Cl. CPC A61B 1/000094; A61B 1/00006; A61B 1/0005; A61B 1/31; A61B 34/25; G06V 20/50; G16H 15/00; G16H 40/63; A61B 2034/2065; G06V 2201/031; G06V 2201/034

(57) ABSTRACT

There are provided an information processing apparatus, an information processing method, an endoscope system, and a report creation support device which can efficiently input information on a site. The information processing apparatus includes a first processor. The first processor acquires an image captured using an endoscope, displays the acquired image in a first region on a screen of a first display unit, detects a specific region in a hollow organ from the acquired image, displays a plurality of sites constituting the hollow organ to which the detected specific region belongs, in a second region on the screen of the first display unit, and accepts selection of one site from among the plurality of sites.

### CROSS-REFERENCE TO RELATED APPLICATIONS

The present application is a Continuation of PCT International Application No. PCT/JP2022/025954 filed on Jun. 29, 2022 claiming priorities under 35 U.S.C § 119(a) to Japanese Patent Application No. 2021-113090 filed on Jul. 7, 2021 and Japanese Patent Application No. 2021-196903 filed on Dec. 3, 2021. Each of the above applications is hereby expressly incorporated by reference, in its entirety, into the present application.

### BACKGROUND OF THE INVENTION

### 1. Field of the Invention

The present invention relates to an information processing apparatus, an information processing method, an endoscope system, and a report creation support device, and particularly relates to an information processing apparatus, an information processing method, an endoscope system, and a report creation support device which process information on an examination (including an observation) by an endoscope.

### 2. Description of the Related Art

In an examination using an endoscope, a report in which findings and the like are described after the examination has ended is created. JP2016-21216A discloses a technique of inputting information necessary for generating a report in real time during the examination. In JP2016-21216A, in a case where a site of a hollow organ is designated by a user during the examination, a disease name selection screen and a characteristic selection screen are displayed in order on a display unit of a tablet terminal that constitutes a findings input support device, and information on the disease name and information on the characteristic selected on each selection screen are recorded in a storage unit in association with information on the designated site of the hollow organ. In JP2016-21216A, the selection of a site is performed on a predetermined selection screen, and is displayed on the display unit according to an examination start instruction and a site selection instruction.

### SUMMARY OF THE INVENTION

However, in JP2016-21216A, it is necessary to call up a site selection screen each time information is input, and thus, there is a disadvantage in that it takes time and effort to acquire the information on the site.

The present invention is made in view of such circumstances, and an object thereof is to provide an information processing apparatus, an information processing method, an endoscope system, and a report creation support device which can efficiently input information on a site.

(1) An information processing apparatus including a first processor, in which the first processor is configured to acquire an image captured using an endoscope, display the acquired image in a first region on a screen of a first display unit, display a plurality of sites of a hollow organ as an observation target in a second region on the screen of the first display unit, and accept selection of one site from among the plurality of sites.

(2) The information processing apparatus described in (1), in which the first processor is configured to detect a specific region of the hollow organ from the acquired image, and display the plurality of sites in the first region in a case where the specific region is detected.

(3) The information processing apparatus described in (2), in which the first processor displays the plurality of sites in the second region in a state where a site to which the detected specific region belongs is selected in advance from among the plurality of sites.

(4) The information processing apparatus described in (1), in which, in a case where a display instruction of the plurality of sites is accepted, the first processor displays the plurality of sites in the first region in a state where one site is selected in advance from among the plurality of sites.

(5) The information processing apparatus described in any one of (1) to (4), in which the first processor displays the plurality of sites in the second region using a schema diagram.

(6) The information processing apparatus described in (5), in which the first processor displays the site being selected such that the site being selected is distinguishable from the other sites, in the schema diagram displayed in the second region.

(7) The information processing apparatus described in any one of (1) to (6), in which the second region is set in a vicinity of a position where a treatment tool appears within the image displayed in the first region.

(8) The information processing apparatus described in (7), in which the first processor displays the second region in an emphasized manner during a first time in a case where selection of the site is accepted.

(9) The information processing apparatus described in any one of (1) to (8), in which the first processor continuously accepts selection of the site after display of the plurality of sites is started.

(10) The information processing apparatus described in any one of (1) to (9), in which the first processor is configured to detect a plurality of specific regions from the acquired image, and execute processing of prompting selection of the site in a case where at least one of the plurality of specific regions is detected.

(11) The information processing apparatus described in any one of (1) to (10), in which the first processor is configured to detect a specific detection target from the acquired image, and execute processing of prompting selection of the site in a case where the detection target is detected.

(12) The information processing apparatus described in (11), in which the detection target is at least one of a lesion part or a treatment tool.

(13) The information processing apparatus described in (12), in which the first processor stops acceptance of the selection of the site during a second time after the detection target is detected.

(14) The information processing apparatus described in any one of (11) to (13), in which the first processor records information on the detection target in association with information on the selected site.

(15) The information processing apparatus described in any one of (9) to (14), in which the first processor displays the second region in an emphasized manner as processing of prompting selection of the site.

(16) The information processing apparatus described in any one of (1) to (15), in which the first processor is configured to detect a treatment tool from the acquired image, choose a plurality of treatment names corresponding to the detected treatment tool, display the plurality of chosen treatment names in a third region on the screen of the first display unit, accept selection of one treatment name from among the plurality of treatment names from start of the display until a third time elapses, and stop acceptance of selection of the site while selection of the treatment name is accepted.

(17) The information processing apparatus described in (16), in which the first processor records information on the selected treatment name in association with information on the selected site.

(18) The information processing apparatus described in any one of (1) to (17), in which the first processor is configured to perform recognition processing on the acquired image, and record a result of the recognition processing in association with information on the selected site.

(19) The information processing apparatus described in (18), in which the first processor performs the recognition processing on the image captured as a static image.

(20) The information processing apparatus described in (19), in which the first processor displays first information indicating the result of the recognition processing in a fourth region on the screen of the first display unit.

(21) The information processing apparatus described in (20), in which the first processor is configured to accept adoption or rejection of the result of the recognition processing indicated by the first information, and record the result of the recognition processing in a case where the result of the recognition processing is adopted.

(22) The information processing apparatus described in (21), in which the first processor accepts only a rejection instruction, and confirms adoption in a case where the rejection instruction is not accepted from start of the display of the first information until a fourth time elapses.

(23) The information processing apparatus described in any one of (18) to (22), in which the first processor is configured to generate second information indicating a result of the recognition processing for each of the sites, and display the second information on the first display unit.

(24) The information processing apparatus described in (23), in which the first processor is configured to divide a first site for which a plurality of the results of the recognition processing are recorded, among the plurality of sites, into a plurality of second sites, and generate the second information indicating the result of the recognition processing for each of the second sites, regarding the first site.

(25) The information processing apparatus described in (24), in which the first processor is configured to set the second sites by equally dividing the first site, and generate the second information by assigning the results of the recognition processing to the second sites in chronological order along an observation direction.

(26) The information processing apparatus described in any one of (23) to (25), in which the first processor generates the second information using a schema diagram.

(27) The information processing apparatus described in any one of (23) to (25), in which the first processor generates the second information using a strip graph divided into a plurality of regions.

(28) The information processing apparatus described in any one of (23) to (27), in which the first processor generates the second information indicating the result of the recognition processing using a color or a density.

(29) The information processing apparatus described in any one of (23) to (28), in which the first processor determines severity of ulcerative colitis using the recognition processing.

(30) The information processing apparatus described in (29), in which the first processor determines the severity of the ulcerative colitis using Mayo Endoscopic Subscore.

(31) The information processing apparatus described in any one of (1) to (30), in which the first processor accepts selection of the site after insertion of the endoscope is detected or after insertion of the endoscope is confirmed by a user input.

(32) The information processing apparatus described in any one of (1) to (31), in which the first processor accepts selection of the site until pulling-out of the endoscope is detected or until pulling-out of the endoscope is confirmed by a user input.

(33) The information processing apparatus described in any one of (1) to (15), in which the first processor is configured to detect a treatment tool from the acquired image, display a plurality of options regarding a treatment target in a fifth region on the screen of the first display unit in a case where the treatment tool is detected from the image, and accept selection of one of the plurality of options displayed in the fifth region.

(34) The information processing apparatus described in (33), in which the plurality of options regarding the treatment target are a plurality of options for a detailed site or a size of the treatment target.

(35) The information processing apparatus described in any one of (1) to (15), in which the first processor is configured to display a plurality of options regarding a region of interest in a fifth region on the screen of the first display unit in a case where a static image to be used in a report is acquired, and accept selection of one of the plurality of options displayed in the fifth region.

(36) The information processing apparatus described in (35), in which the plurality of options regarding the region of interest are a plurality of options for a detailed site or a size of the region of interest.

(37) The information processing apparatus described in any one of (1) to (36), in which the first processor records a captured static image in association with information on the selected site.

(38) The information processing apparatus described in (37), in which the first processor records, as a candidate for an image to be used in a report or a diagnosis, the captured static image in association with the information on the selected site.

(39) The information processing apparatus described in (38), in which the first processor acquires, as the candidate for the image to be used in the report or the diagnosis, a most recent static image in terms of time among static images captured before a time point when selection of the site is accepted, or an oldest static image in terms of time among static images captured after the time point when the selection of the site is accepted.

(40) A report creation support device that supports creation of a report, including a second processor, in which the second processor is configured to display a report creation screen with at least an input field for a site, on a second display unit, acquire information on the site selected in the information processing apparatus described in any one of (1) to (39), automatically input the acquired information on the site to the input field for the site, and accept correction of the automatically input information of the input field for the site.

(41) A report creation support device that supports creation of a report, including a second processor, in which the second processor is configured to display a report creation screen with at least input fields for a site and a static image, on a second display unit, acquire information on the site and the static image selected in the information processing apparatus described in any one of (37) to (39), automatically input the acquired information on the site to the input field for the site, automatically input the acquired static image to the input field for the static image, and accept correction of the automatically input information of the input field for the site and the automatically input static image of the input field for the static image.

(42) The report creation support device described in (39), in which the second processor displays the input field for the site such that the input field for the site is distinguishable from other input fields on the report creation screen.

(43) An endoscope system including an endoscope; the information processing apparatus described in any one of (1) to (39); and an input device.

(44) An information processing method including a step of acquiring an image captured using an endoscope; a step of displaying the acquired image in a first region on a screen of a first display unit; a step of detecting a specific region in a hollow organ from the acquired image; a step of displaying a plurality of sites constituting the hollow organ to which the detected specific region belongs, in a second region on the screen of the first display unit; and a step of accepting selection of one site from among the plurality of sites.

According to the present invention, it is possible to efficiently input information on a site.

### BRIEF DESCRIPTION OF THE DRAWINGS

FIG. 1 is a block diagram illustrating an example of a system configuration of an endoscopic image diagnosis support system.

FIG. 2 is a block diagram illustrating an example of a system configuration of an endoscope system.

FIG. 3 is a block diagram illustrating a schematic configuration of an endoscope.

FIG. 4 is a diagram illustrating an example of a configuration of an end face of a distal end portion.

FIG. 5 is a diagram illustrating an example of an endoscopic image in a case where a treatment tool is used.

FIG. 6 is a block diagram of main functions of a processor device.

FIG. 7 is a block diagram of main functions of an endoscopic image processing device.

FIG. 8 is a block diagram of main functions of an image recognition processing unit.

FIG. 9 is a diagram illustrating an example of display of a screen during an examination.

FIG. 10 is a diagram illustrating another example of display of a screen during an examination.

FIG. 11 is a diagram illustrating an example of a site selection box.

FIGS. 12A to 12C are diagrams illustrating examples of display of a site being selected.

FIG. 13 is a diagram illustrating an example of a display position of a site selection box.

FIG. 14 is a diagram illustrating an example of emphasized display of a site selection box.

FIGS. 15A and 15B are diagrams illustrating examples of a treatment tool detection icon.

FIG. 16 is a diagram illustrating an example of a display position of a treatment tool detection icon.

FIGS. 17A and 17B are diagrams illustrating examples of a treatment name selection box.

FIG. 18 is a diagram illustrating an example of a table.

FIG. 19 is a diagram illustrating an example of a display position of a treatment name selection box.

FIG. 20 is a diagram illustrating an example of a progress bar.

FIG. 21 is a diagram illustrating an example of display of a screen immediately after selection processing of a treatment name is performed.

FIG. 22 is a diagram illustrating an example of display of a screen immediately after acceptance of selection of a treatment name is ended.

FIG. 23 is a block diagram illustrating an example of a system configuration of an endoscope information management system.

FIG. 24 is a block diagram of main functions of an endoscope information management device.

FIG. 25 is a block diagram of main functions of a report creation support unit.

FIG. 26 is a diagram illustrating an example of a selection screen.

FIG. 27 is a diagram illustrating an example of a detailed input screen.

FIG. 28 is a diagram illustrating an example of display of a drop-down list.

FIG. 29 is a diagram illustrating an example of a detailed input screen which is automatically filled.

FIG. 30 is a diagram illustrating an example of a detailed input screen during correction.

FIG. 31 is a diagram illustrating an example of a detailed input screen after an input is completed.

FIG. 32 is a flowchart illustrating a procedure of processing of accepting an input of a site.

FIG. 33 is a flowchart illustrating a procedure of processing of accepting an input of a treatment name.

FIG. 34 is a flowchart illustrating a procedure of processing of accepting an input of a treatment name.

FIG. 35 is a diagram illustrating a modification example of a detailed input screen.

FIG. 36 is a diagram illustrating an example of display of a detailed site selection box.

FIG. 37 is a diagram illustrating an example of a detailed site selection box.

FIG. 38 is a diagram illustrating an example of a size selection box.

FIG. 39 is a block diagram of main functions of an image recognition processing unit.

FIG. 40 is a diagram illustrating an example of display of a screen before an endoscope is inserted.

FIG. 41 is a diagram illustrating an example of display of a screen in a case where insertion of an endoscope is detected.

FIG. 42 is a diagram illustrating an example of display of a screen in a case where detection of insertion of an endoscope is confirmed.

FIG. 43 is a diagram illustrating an example of display of a screen after detection of insertion of an endoscope is confirmed.

FIG. 44 is a diagram illustrating an example of display of a screen in a case where an ileocecum being reached is manually input.

FIG. 45 is a diagram illustrating an example of display of a screen in a case where the ileocecum being reached is confirmed.

FIG. 46 is a diagram illustrating an example of display of a screen in a case where pulling out an endoscope is detected.

FIG. 47 is a diagram illustrating an example of display of a screen in a case where detection of pulling out an endoscope is confirmed.

FIG. 48 is a diagram illustrating a list of icons displayed on a screen.

FIG. 49 is a diagram illustrating an example of switching information displayed at a display position of a site selection box.

FIG. 50 is a block diagram of functions of an endoscopic image processing device for recording and outputting a result of recognition processing.

FIG. 51 is a block diagram of main functions of an image recognition processing unit.

FIG. 52 is a diagram illustrating an example of a site selection box.

FIG. 53 is a diagram illustrating an example of map data.

FIG. 54 is a flowchart illustrating a procedure of selection processing of a site.

FIG. 55 is a diagram illustrating an outline of recording processing of Mayo score.

FIG. 56 is a flowchart illustrating a procedure of processing of determining Mayo score and adopting or rejecting results.

FIG. 57 is a diagram illustrating an example of display of a determination result of Mayo score.

FIG. 58 is a diagram illustrating a temporal change of display of a Mayo score display box.

FIG. 59 is a diagram illustrating an example of display of map data.

FIG. 60 is a diagram illustrating an example of map data in a case where a plurality of Mayo scores are recorded for one site.

FIG. 61 is a diagram illustrating another example of map data.

FIG. 62 is a diagram illustrating another example of map data.

FIG. 63 is a diagram illustrating another example of map data.

FIG. 64 is a diagram illustrating an example of presentation of map data.

### DESCRIPTION OF THE PREFERRED EMBODIMENTS

Hereinafter, preferred embodiments of the present invention will be described in detail with reference to the accompanying drawings.

### First Embodiment

[Endoscopic Image Diagnosis Support System]

Here, a case where the present invention is applied to an endoscopic image diagnosis support system will be described as an example. The endoscopic image diagnosis support system is a system that supports detection and discrimination of a lesion or the like in an endoscopy. In the following, an example of application to an endoscopic image diagnosis support system that supports detection and discrimination of a lesion and the like in a lower digestive tract endoscopy (large intestine examination) will be described.

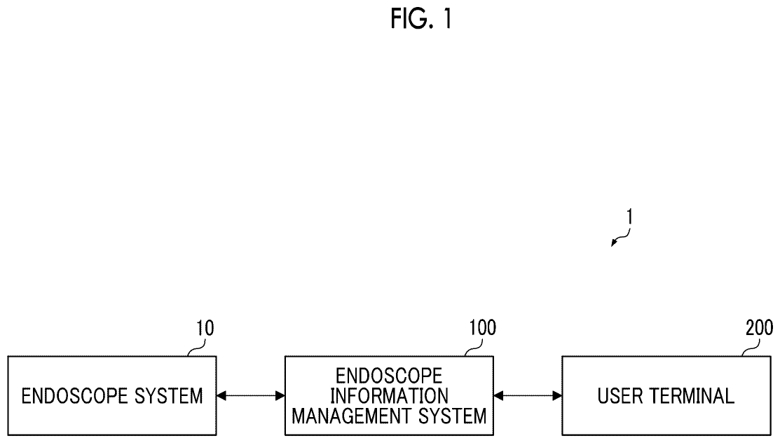

FIG. 1 is a block diagram illustrating an example of a system configuration of the endoscopic image diagnosis support system.

As illustrated in the figure, an endoscopic image diagnosis support system 1 of the present embodiment has an endoscope system 10, an endoscope information management system 100, and a user terminal 200.

[Endoscope System]

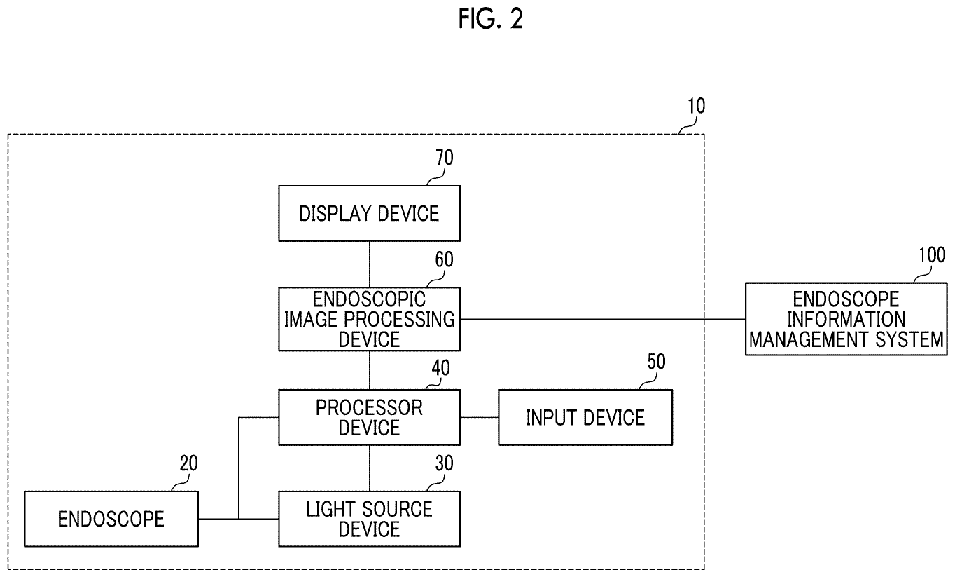

FIG. 2 is a block diagram illustrating an example of a system configuration of the endoscope system.

The endoscope system 10 of the present embodiment is configured as a system capable of an observation using special light (special light observation) in addition to an observation using white light (white light observation). In the special light observation, a narrow-band light observation is included. In the narrow-band light observation, a blue laser imaging observation (BLI observation), a narrow band imaging observation (NBI observation), a linked color imaging observation (LCI observation), and the like are included. Note that the special light observation itself is a well-known technique, so detailed description thereof will be omitted.

As illustrated in FIG. 2, the endoscope system 10 of the present embodiment has an endoscope 20, a light source device 30, a processor device 40, an input device 50, an endoscopic image processing device 60, a display device 70, and the like.

[Endoscope]

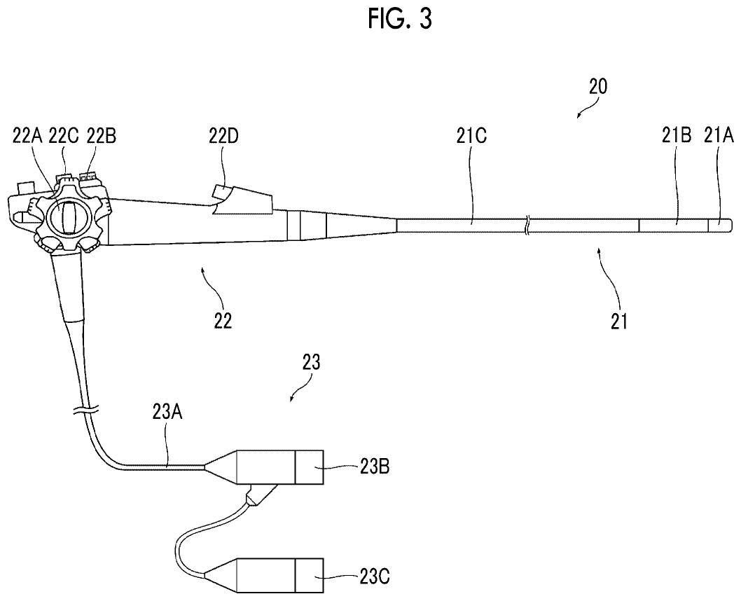

FIG. 3 is a diagram illustrating a schematic configuration of the endoscope.

The endoscope 20 of the present embodiment is an endoscope for a lower digestive organ. As illustrated in FIG. 3, the endoscope 20 is a flexible endoscope (electronic endoscope), and has an insertion part 21, an operation part 22, and a connection part 23.

The insertion part 21 is a part to be inserted into a hollow organ (large intestine in the present embodiment). The insertion part 21 includes a distal end portion 21A, a bendable portion 21B, and a soft portion 21C in order from a distal end.

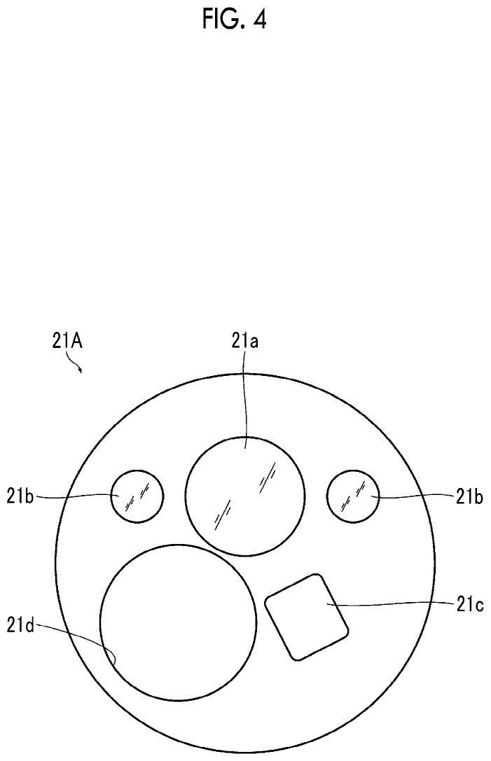

FIG. 4 is a diagram illustrating an example of a configuration of an end face of the distal end portion.

As illustrated in the figure, in the end face of the distal end portion 21A, an observation window 21a, illumination windows 21b, an air/water supply nozzle 21c, a forceps outlet 21d, and the like are provided. The observation window 21a is a window for an observation. The inside of the hollow organ is imaged through the observation window 21a. Imaging is performed via an optical system and an image sensor (not illustrated) built in the distal end portion 21A. As the image sensor, for example, a complementary metal-oxide-semiconductor image sensor (CMOS image sensor), a charge-coupled device image sensor (CCD image sensor), or the like is used. The illumination windows 21b are windows for illumination. The inside of the hollow organ is irradiated with illumination light via the illumination windows 21b. The air/water supply nozzle 21c is a nozzle for cleaning. A cleaning liquid and a drying gas are sprayed from the air/water supply nozzle 21c toward the observation window 21a. The forceps outlet 21d is an outlet for a treatment tool such as forceps. The forceps outlet 21d functions as a suction port for sucking body fluids and the like.

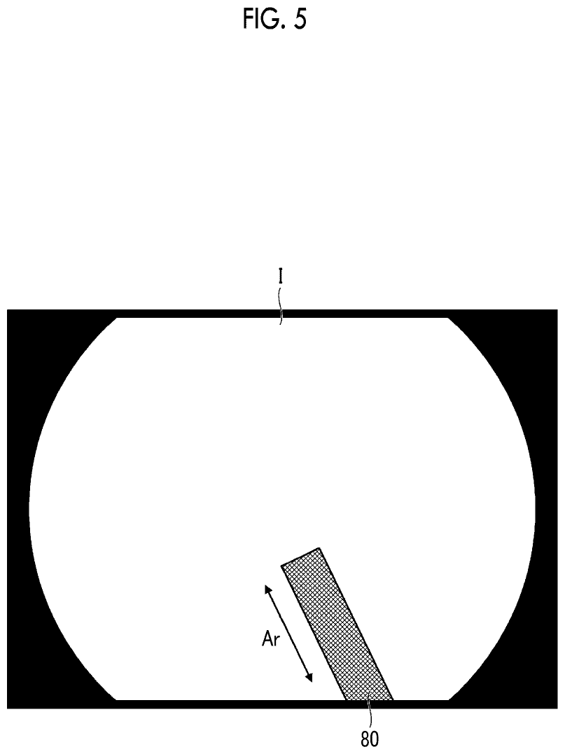

FIG. 5 is a diagram illustrating an example of an endoscopic image in a case where a treatment tool is used.

A position of the forceps outlet 21d is fixed with respect to a position of the observation window 21a. Therefore, in a case where a treatment tool is used, the treatment tool always appears from a certain position in the image, and is taken in and out along a certain direction. FIG. 5 illustrates an example of a case in which a treatment tool 80 appears from a lower right position in an endoscopic image I, and is moved along a direction (forceps direction) indicated by an arrow Ar.

The bendable portion 21B is a portion that is bent according to an operation of an angle knob 22A of the operation part 22. The bendable portion 21B is bent in four directions of up, down, left, and right.

The soft portion 21C is an elongated portion provided between the bendable portion 21B and the operation part 22. The soft portion 21C has flexibility.

The operation part 22 is a part that is held by a user (operator) to perform various operations. The operation part 22 includes various operation members. As an example, the operation part 22 includes the angle knob 22A for a bending operation of the bendable portion 21B, an air/water supply button 22B for performing an air/water supply operation, and a suction button 22C for performing a suction operation. In addition, the operation part 22 includes an operation member (shutter button) for imaging a static image, an operation member for switching an observation mode, an operation member for switching on and off of various support functions, and the like. Further, the operation part 22 includes a forceps insertion port 22D for inserting a treatment tool such as forceps. The treatment tool inserted from the forceps insertion port 22D is drawn out from the forceps outlet 21d (refer to FIG. 4) on the distal end of the insertion part 21. As an example, the treatment tool includes biopsy forceps, snares, and the like.

The connection part 23 is a part for connecting the endoscope 20 to the light source device 30, the processor device 40, and the like. The connection part 23 includes a cord 23A extending from the operation part 22, and a light guide connector 23B and a video connector 23C that are provided on the distal end of the cord 23A. The light guide connector 23B is a connector for connecting the endoscope 20 to the light source device 30. The video connector 23C is a connector for connecting the endoscope 20 to the processor device 40.

[Light Source Device]

The light source device 30 generates illumination light. As described above, the endoscope system 10 of the present embodiment is configured as a system capable of the special light observation in addition to the normal white light observation. Therefore, the light source device 30 is configured to be capable of generating light (for example, narrow-band light) corresponding to the special light observation in addition to the normal white light. Note that, as described above, the special light observation itself is a well-known technique, so the description for the light generation will be omitted.

[Processor Device]

The processor device 40 integrally controls the operation of the entire endoscope system. The processor device 40 includes, as a hardware configuration, a processor, a main storage unit, an auxiliary storage unit, a communication unit, an operation unit, and the like. That is, the processor device 40 has a so-called computer configuration as the hardware configuration. For example, the processor is configured by a central processing unit (CPU) and the like. For example, the main storage unit is configured by a random-access memory (RAM) and the like. For example, the auxiliary storage unit is configured by a flash memory and the like. For example, the operation unit is configured by an operation panel including an operation button and the like.

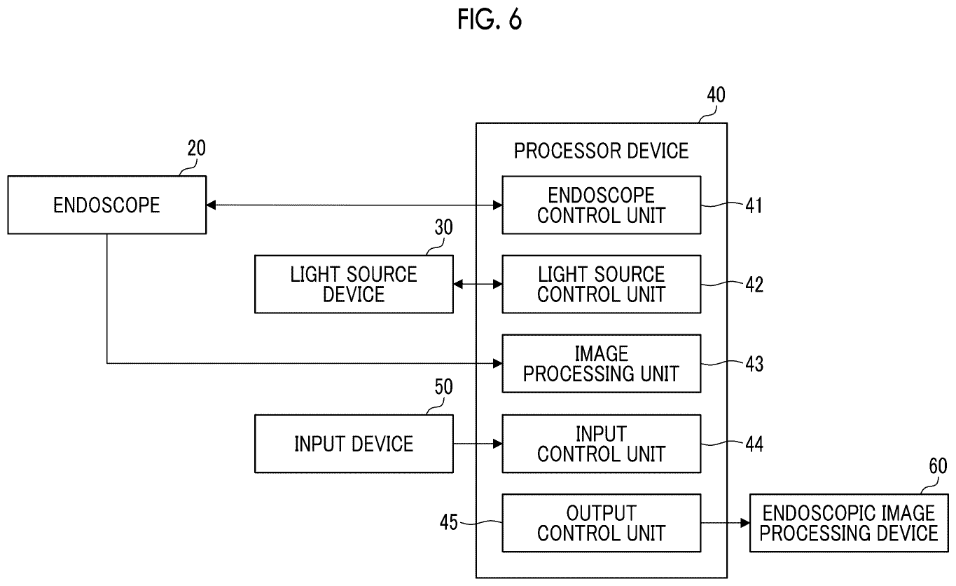

FIG. 6 is a block diagram of main functions of the processor device.

As illustrated in the figure, the processor device 40 has functions of an endoscope control unit 41, a light source control unit 42, an image processing unit 43, an input control unit 44, an output control unit 45, and the like. Each function is realized by the processor executing a predetermined program. The auxiliary storage unit stores various programs executed by the processor, various kinds of data necessary for control, and the like.

The endoscope control unit 41 controls the endoscope 20. The control for the endoscope 20 includes image sensor drive control, air/water supply control, suction control, and the like.

The light source control unit 42 controls the light source device 30. The control for the light source device 30 includes light emission control for a light source, and the like.

The image processing unit 43 performs various kinds of signal processing on signals output from the image sensor of the endoscope 20 to generate a captured image (endoscopic image).

The input control unit 44 accepts an input of an operation and an input of various kinds of information via the input device 50.

The output control unit 45 controls an output of information to the endoscopic image processing device 60. The information to be output to the endoscopic image processing device 60 includes various kinds of operation information input from the input device 50, and the like in addition to the endoscopic image obtained by imaging.

[Input Device]

The input device 50 constitutes a user interface in the endoscope system 10 together with the display device 70. For example, the input device 50 is configured by a keyboard, a mouse, a foot switch, and the like. The foot switch is an operation device that is placed at the feet of the user (operator) and that is operated with the foot. The foot switch outputs a predetermined operation signal in a case of stepping on a pedal. In addition, the input device 50 can include a known input device such as a touch panel, an audio input device, and a gaze input device. Further, the input device 50 can include an operation panel provided in the processor device.

[Endoscopic Image Processing Device]

The endoscopic image processing device 60 performs processing of outputting the endoscopic image to the display device 70. Further, the endoscopic image processing device 60 performs various kinds of recognition processing on the endoscopic image as necessary, and performs processing of outputting the result to the display device 70. The recognition processing includes processing of detecting a lesion part or the like, discrimination processing for the detected lesion part or the like, processing of detecting a specific region in a hollow organ, processing of detecting a treatment tool, and the like. Moreover, the endoscopic image processing device 60 performs processing of supporting an input of information necessary for creating a report during the examination. Further, the endoscopic image processing device 60 performs processing of communicating with the endoscope information management system 100, and outputting examination information or the like to the endoscope information management system 100. The endoscopic image processing device 60 is an example of an information processing apparatus.

The endoscopic image processing device 60 includes, as a hardware configuration, a processor, a main storage unit, an auxiliary storage unit, a communication unit, and the like. That is, the endoscopic image processing device 60 has a so-called computer configuration as the hardware configuration. For example, the processor is configured by a CPU and the like. The processor of the endoscopic image processing device 60 is an example of a first processor. For example, the main storage unit is configured by a RAM and the like. For example, the auxiliary storage unit is configured by a flash memory and the like. For example, the communication unit is configured by a communication interface connectable to a network. The endoscopic image processing device 60 is communicably connected to the endoscope information management system 100 via the communication unit.

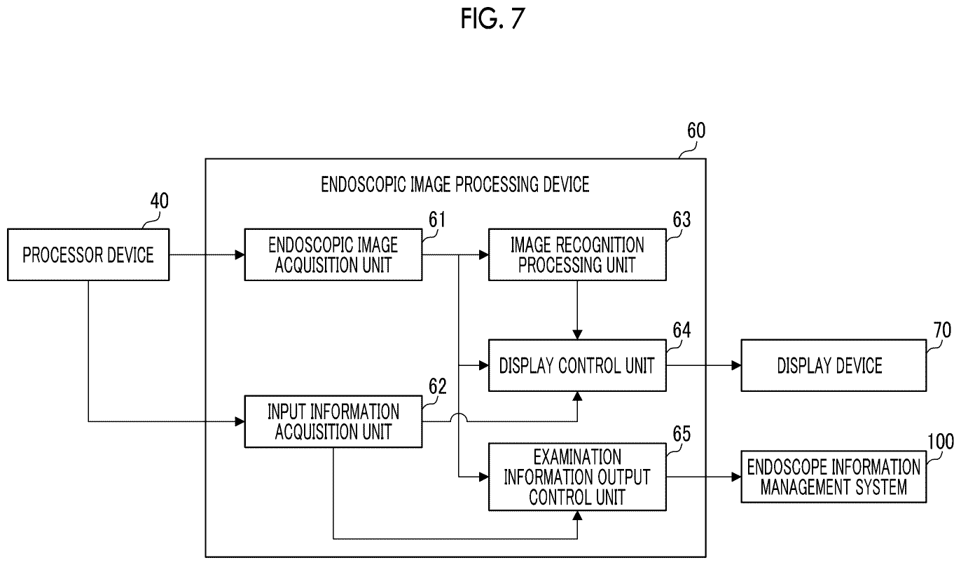

FIG. 7 is a block diagram of main functions of the endoscopic image processing device.

As illustrated in the figure, the endoscopic image processing device 60 mainly has functions of an endoscopic image acquisition unit 61, an input information acquisition unit 62, an image recognition processing unit 63, a display control unit 64, an examination information output control unit 65, and the like. Each function is realized by the processor executing a predetermined program. The auxiliary storage unit stores various programs executed by the processor, various kinds of data necessary for control, and the like.

[Endoscopic Image Acquisition Unit]

The endoscopic image acquisition unit 61 acquires an endoscopic image from the processor device 40. Acquisition of an image is performed in real time. That is, the image captured by the endoscope is acquired in real time.

[Input Information Acquisition Unit]

The input information acquisition unit 62 acquires information input via the input device 50 or the endoscope 20. The information input via the input device 50 includes information input via a keyboard, a mouse, a foot switch, or the like. Further, the information input via the endoscope 20 includes information such as an imaging instruction for a static image. As described below, in the present embodiment, a selection operation of a site and a selection operation of a treatment name are performed via the foot switch. The input information acquisition unit 62 acquires operation information of the foot switch via the processor device 40.

[Image Recognition Processing Unit]

The image recognition processing unit 63 performs various kinds of recognition processing on the endoscopic image acquired by the endoscopic image acquisition unit 61. The recognition processing is performed in real time. That is, the recognition processing is performed in real time on the image captured by the endoscope.

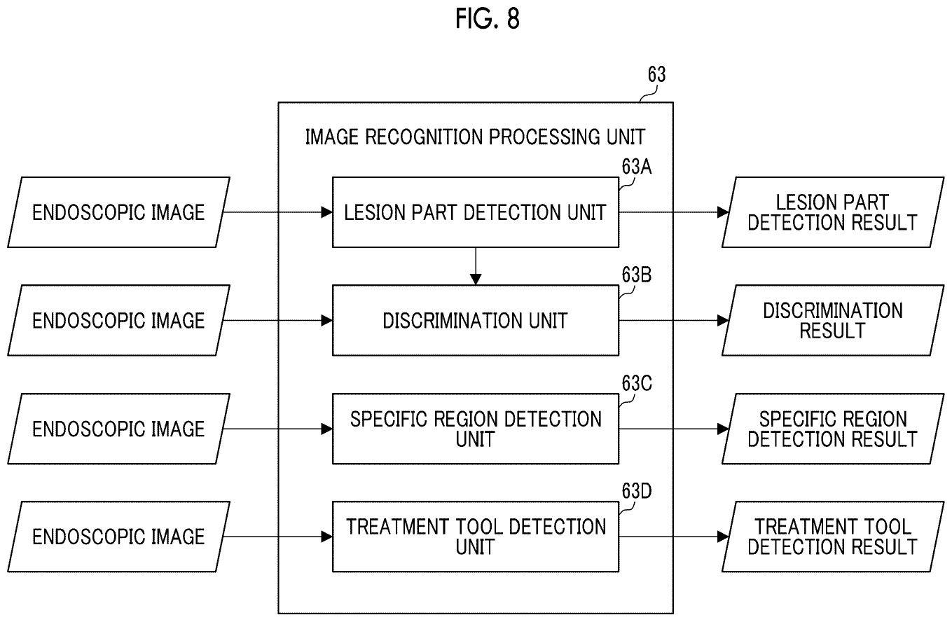

FIG. 8 is a block diagram of main functions of the image recognition processing unit.

As illustrated in the figure, the image recognition processing unit 63 has functions of a lesion part detection unit 63A, a discrimination unit 63B, a specific region detection unit 63C, a treatment tool detection unit 63D, and the like.

The lesion part detection unit 63A detects a lesion part such as a polyp from the endoscopic image. The processing of detecting the lesion part includes processing of detecting a part with a possibility of a lesion (benign tumor, dysplasia, or the like), processing of recognizing a part with features that may be directly or indirectly associated with a lesion (erythema or the like), and the like in addition to processing of detecting a part that is definitely a lesion part.

The discrimination unit 63B performs the discrimination processing on the lesion part detected by the lesion part detection unit 63A. As an example, in the present embodiment, neoplastic or non-neoplastic (hyperplastic) discrimination processing is performed on the lesion part such as a polyp detected by the lesion part detection unit 63A.

The specific region detection unit 63C performs processing of detecting a specific region in the hollow organ from the endoscopic image. For example, processing of detecting an ileocecum of the large intestine or the like is performed. The large intestine is an example of the hollow organ. The ileocecum is an example of the specific region. The specific region detection unit 63C may detect, as the specific region, a hepatic flexure (right colon), a splenic flexure (left colon), a rectosigmoid, and the like in addition to the ileocecum. Further, the specific region detection unit 63C may detect a plurality of specific regions.

The treatment tool detection unit 63D performs processing of detecting a treatment tool appearing in the image from the endoscopic image, and discriminating the type of the treatment tool. The treatment tool detection unit 63D can be configured to detect a plurality types of treatment tools such as biopsy forceps, snares, and hemostatic clips.

Each unit (the lesion part detection unit 63A, the discrimination unit 63B, the specific region detection unit 63C, the treatment tool detection unit 63D, and the like) constituting the image recognition processing unit 63 is configured by, for example, artificial intelligence (AI) having a learning function. Specifically, each unit is configured by AI or a trained model trained using deep learning or a machine learning algorithm such as a neural network (NN), a convolutional neural network (CNN), AdaBoost, and random forest.

Note that a part or all of the units constituting the image recognition processing unit 63 can be configured to calculate a feature amount from the image and to perform detection or the like using the calculated feature amount, instead of being configured by AI or the trained model.

[Display Control Unit]

The display control unit 64 controls display of the display device 70. In the following, main display control performed by the display control unit 64 will be described.

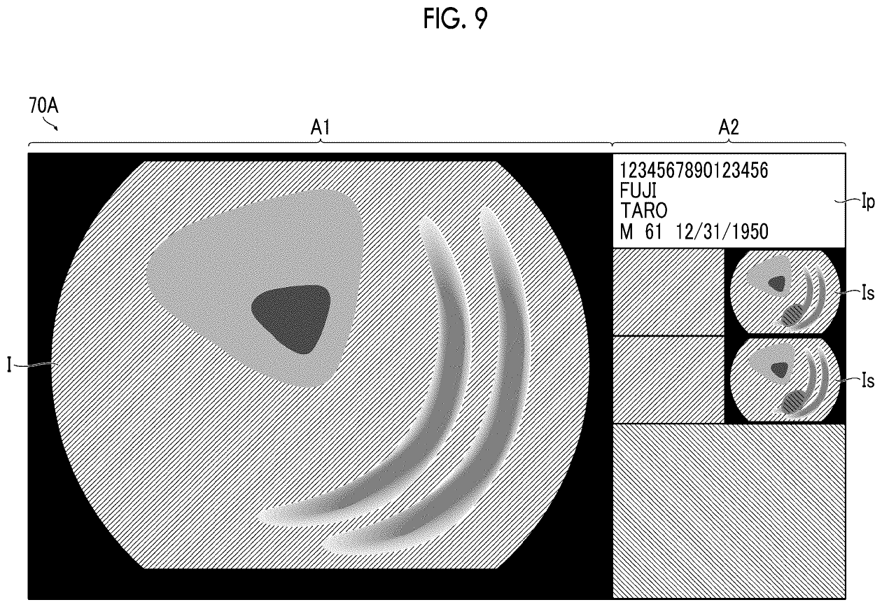

The display control unit 64 displays the image (endoscopic image) captured by the endoscope 20 on the display device 70 in real time during the examination. FIG. 9 is a diagram illustrating an example of display of a screen during the examination. As illustrated in the figure, the endoscopic image I (live view) is displayed in a main display region A1 set on a screen 70A. The main display region A1 is an example of a first region. A secondary display region A2 is further set on the screen 70A, and various kinds of information regarding the examination are displayed. In the example illustrated in FIG. 9, a case where information Ip regarding a patient and a static image Is of the endoscopic image captured during the examination are displayed in the secondary display region A2 is illustrated. For example, the static images Is are displayed in the captured order from top to bottom of the screen 70A.

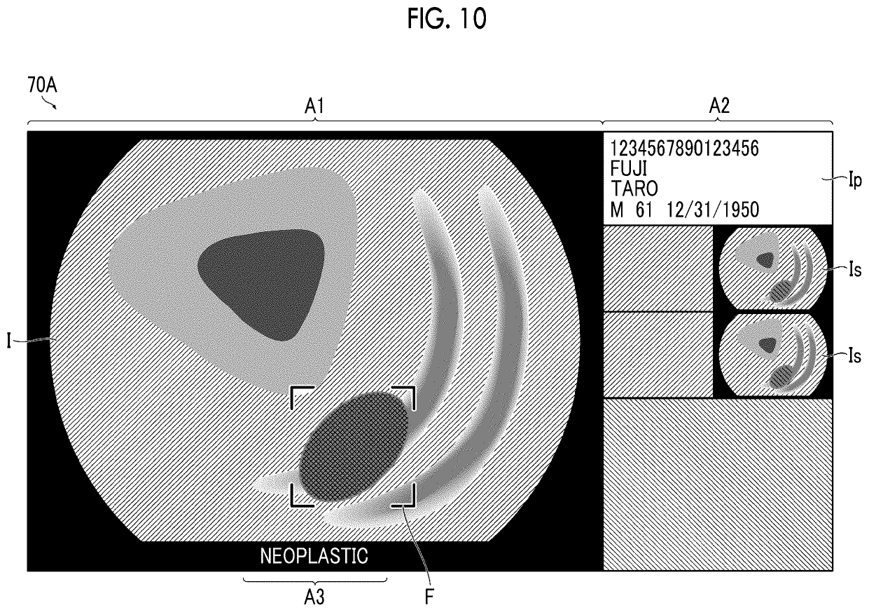

FIG. 10 is a diagram illustrating another example of display of the screen during the examination. The figure illustrates an example of display of a screen in a case where a detection support function for a lesion part is ON.

As illustrated in the figure, in a case where the detection support function for a lesion part is ON, in a case where a lesion part P is detected from the endoscopic image I being displayed, the display control unit 64 displays the endoscopic image I on the screen 70A by enclosing a target region (region of the lesion part P) with a frame F. Moreover, in a case where a discrimination support function is ON, the display control unit 64 displays a discrimination result in a discrimination result display region A3 set on the screen 70A in advance. In the example illustrated in FIG. 10, a case where the discrimination result is “NEOPLASTIC” is illustrated.

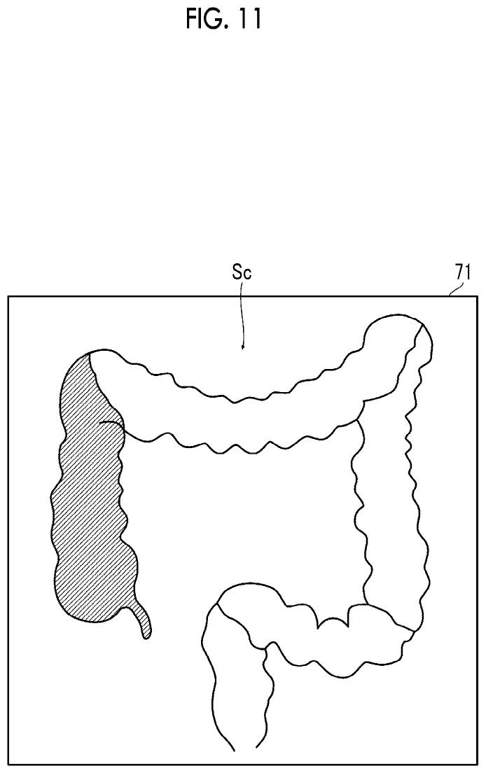

Further, in a case where a specific condition is satisfied, the display control unit 64 displays a site selection box 71 on the screen 70A. The site selection box 71 is a region for selecting a site of the hollow organ under the examination, on the screen. The user can select a site under observation (site being imaged by the endoscope) with the site selection box 71. The site selection box 71 constitutes an interface for inputting a site on the screen. In the present embodiment, as the site selection box, a box for selecting a site of the large intestine is displayed on the screen 70A. FIG. 11 is a diagram illustrating an example of the site selection box. As illustrated in the figure, in the present embodiment, a schema diagram Sc of the large intestine is displayed in a box defined by a frame with a rectangular outer shape, and the selection of a site is accepted on the schema diagram Sc. In the example illustrated in FIG. 11, a case where the large intestine is selected from three sites is illustrated. Specifically, a case where the large intestine is selected from three sites of “ASCENDING COLON”, “TRANSVERSE COLON”, and “DESCENDING COLON” is illustrated. In the example, the ascending colon is classified including a cecum. Note that FIG. 11 is an example of division of sites, and it is also possible to divide and select the sites in more detail.

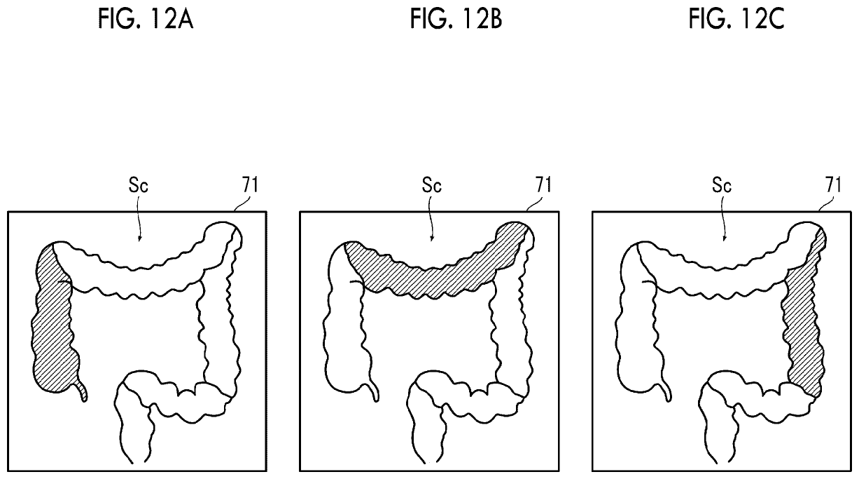

FIGS. 12A to 12C are diagrams illustrating examples of display of the site being selected. FIG. 12A illustrates an example of a case in which the “ascending colon” is selected. FIG. 12B illustrates an example of a case in which the “transverse colon” is selected. FIG. 12C illustrates an example of a case in which the “descending colon” is selected. As illustrated in each diagram of FIGS. 12A to 12C, in the schema diagram Sc, the selected site is displayed to be distinguishable from the other sites. In the example illustrated in FIGS. 12A to 12C, the selected site is displayed to be distinguishable from the other sites by changing the color of the selected site. In addition, the selected site may be distinguished from the other sites by making the selected site blink or the like.

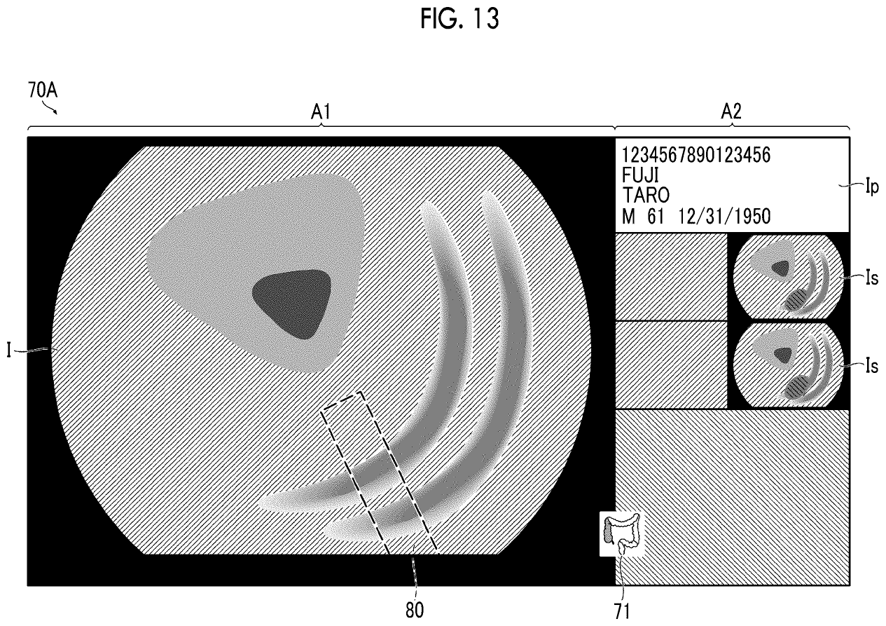

FIG. 13 is a diagram illustrating an example of a display position of the site selection box. The site selection box 71 is displayed at a fixed position on the screen 70A. The position where the site selection box 71 is displayed is set in the vicinity of the position where the treatment tool 80 appears in the endoscopic image displayed in the main display region A1. As an example, the position is set to a position that does not overlap the endoscopic image I displayed in the main display region A1 and that is adjacent to the position where the treatment tool 80 appears. The position is a position in substantially the same direction as the direction in which the treatment tool 80 appears, with respect to a center of the endoscopic image I displayed in the main display region A1. In the present embodiment, as illustrated in FIG. 13, the treatment tool 80 is displayed from the lower right position of the endoscopic image I displayed in the main display region A1. Thus, the position where the site selection box 71 is displayed is set to the lower right position with respect to the center of the endoscopic image I displayed in the main display region A1. The region where the site selection box 71 is displayed on the screen 70A is an example of a second region.

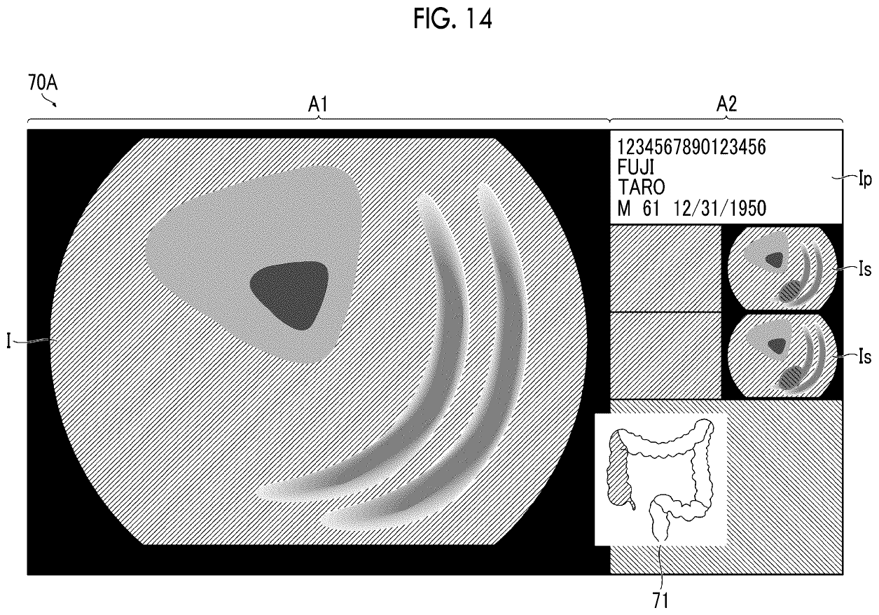

In a case where the site is selected, the display control unit 64 displays the site selection box 71 in an emphasized manner for a fixed time (time T1). FIG. 14 is a diagram illustrating an example of emphasized display of the site selection box. As illustrated in the figure, in the present embodiment, the site selection box 71 is displayed in an emphasized manner by being enlarged. In addition, as the emphasizing method, methods such as changing a color from a normal display form, enclosing with a frame, and blinking, and a method of a combination thereof can be adopted. The method of selecting the site will be described below.

Note that, in the endoscope system 10 of the present embodiment, in a case where the site selection box 71 is first displayed on the screen 70A, the site selection box 71 is displayed on the screen 70A in a state where one site is selected in advance.

Here, in the present embodiment, the condition for displaying the site selection box 71 on the screen 70A is a case where the specific region is detected by the specific region detection unit 63C. In the present embodiment, in a case where the ileocecum is detected as the specific region, the site selection box 71 is displayed on the screen 70A. In this case, the display control unit 64 displays the site selection box 71 on the screen 70A in a state where a site to which the specific region belongs is selected in advance. For example, in a case where the specific region is the ileocecum, the site selection box 71 is displayed on the screen in a state where the ascending colon is selected (refer to FIG. 12A). Further, for example, in a case where the specific region is the hepatic flexure, the site selection box 71 may be displayed on the screen in a state where the transverse colon is selected, and in a case where the specific region is the splenic flexure, the site selection box 71 may be displayed on the screen in a state where the descending colon is selected.

In this manner, in a case where the site selection box 71 is displayed on the screen 70A with the detection of the specific region as a trigger, by displaying the site selection box 71 on the screen 70A in a state where the site to which the specific region belongs is selected in advance, it is possible to save time and effort for selecting a site. Accordingly, it is possible to efficiently input information on the site.

In general, the user (operator) ascertains the position of the distal end portion 21A of the endoscope during the examination from an insertion length of the endoscope, the image during the examination, the feel during operation in the endoscope operation, and the like. With the endoscope system 10 of the present embodiment, in a case where the user determines that the site selected in advance is different from the actual site, the user can correct the selected site. On the other hand, in a case where the user determines that the site selected in advance is correct, the selection operation by the user is not necessary. Accordingly, it is possible to save the user's time and effort, and to accurately input information on the site. Further, information on an appropriate site can be associated with the endoscopic image, lesion information acquired during the examination, treatment information during the examination, and the like.

Moreover, it is possible to save the user's time and effort and to input information on an appropriate site by adopting a configuration in which, for a site including a region (for example, ileocecum) that can be detected with high accuracy by the specific region detection unit 63C, the site is selected in advance, and for a site (for example, transverse colon) not including a region that can be detected with high accuracy by the specific region detection unit 63C, the selection from the user is accepted without selecting the site in advance.

Note that, in a case where the site selection box 71 is displayed on the screen 70A in a state where a specific site is selected in advance, in a case where the site selection box 71 is first displayed on the screen 70A, the display control unit 64 displays the site selection box 71 in an emphasized manner for a fixed time (time T1) (refer to FIG. 14).

Time T1 for which the site selection box 71 is displayed in an emphasized manner is determined in advance. Time T1 may be arbitrarily set by the user. Time T1 for which the site selection box 71 is displayed in an emphasized manner is an example of a first time.

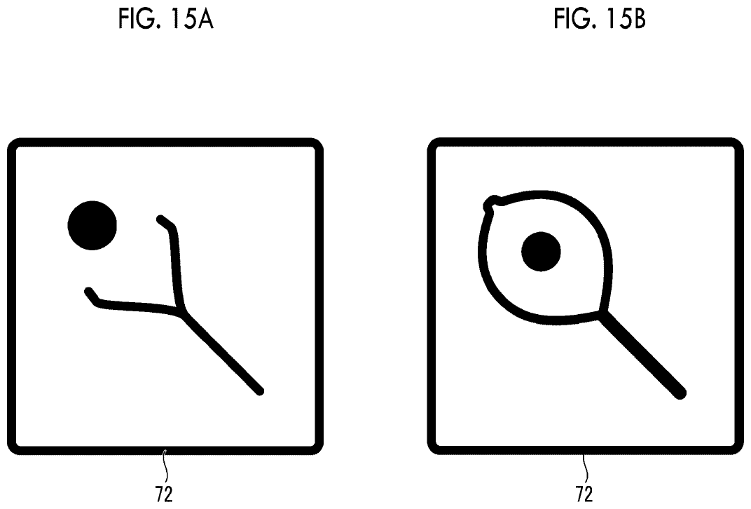

In a case where the treatment tool is detected, the display control unit 64 displays an icon indicating detection of the treatment tool (hereinafter, referred to as a “treatment tool detection icon”) on the screen 70A. FIGS. 15A and 15B are diagrams illustrating examples of the treatment tool detection icon. As illustrated in the figures, different icons are used for detected treatment tools, respectively. FIG. 15A is a diagram illustrating an example of a treatment tool detection icon 72 displayed in a case where biopsy forceps are detected. FIG. 15B is a diagram illustrating an example of the treatment tool detection icon 72 displayed in a case where a snare is detected. A symbol depicting the corresponding treatment tool is used as the treatment tool detection icon in each figure. In addition, the treatment tool detection icon can be represented graphically.

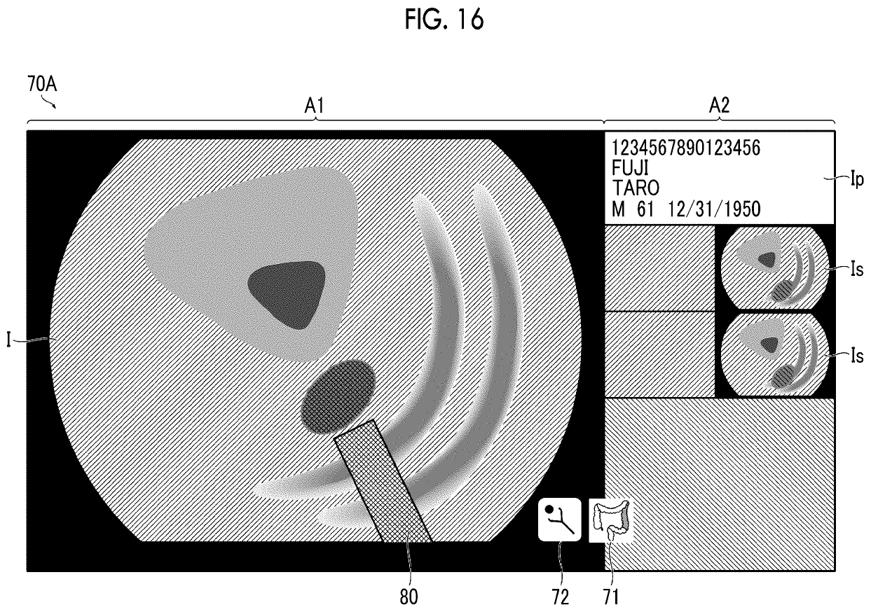

FIG. 16 is a diagram illustrating an example of the display position of the treatment tool detection icon. The treatment tool detection icon 72 is displayed at a fixed position on the screen 70A. The position where the treatment tool detection icon 72 is displayed is set in the vicinity of the position where the treatment tool 80 appears in the endoscopic image I displayed in the main display region A1. As an example, the position is set to a position that does not overlap the endoscopic image I displayed in the main display region A1 and that is adjacent to the position where the treatment tool 80 appears. The position is a position in substantially the same direction as the direction in which the treatment tool 80 appears, with respect to the center of the endoscopic image I displayed in the main display region A1. In the present embodiment, as illustrated in FIG. 16, the treatment tool 80 is displayed from the lower right position of the endoscopic image I displayed in the main display region A1. Thus, the position where the treatment tool detection icon 72 is displayed is set to the lower right position with respect to the center of the endoscopic image I displayed in the main display region A1. In a case where the site selection box 71 is simultaneously displayed, the treatment tool detection icon 72 is displayed side by side with the site selection box 71. In this case, the treatment tool detection icon 72 is displayed at a position closer to the treatment tool 80 than the site selection box 71.

In this manner, by displaying the treatment tool detection icon 72 in the vicinity of the position where the treatment tool 80 appears in the endoscopic image I, the user can easily recognize that the treatment tool 80 has been detected (recognized) from the endoscopic image I. That is, it is possible to improve visibility.

Moreover, in a case where a specific condition is satisfied, the display control unit 64 displays a treatment name selection box 73 on the screen 70A. The treatment name selection box 73 is a region for selecting one treatment name from among a plurality of treatment names (specimen collection methods in a case of specimen collection) on the screen. The treatment name selection box 73 constitutes an interface for inputting the treatment name on the screen. In the present embodiment, the treatment name selection box 73 is displayed after the treatment has ended. The end of the treatment is determined on the basis of the detection result of the treatment tool detection unit 63D. Specifically, in a case where the treatment tool 80 appearing in the endoscopic image I disappears from the endoscopic image I, and a fixed time (time T2) has elapsed from the disappearance, it is determined that the treatment has ended. For example, time T2 is 15 seconds. Time T2 may be arbitrarily set by the user. Time T2 is an example of the first time. By displaying the treatment name selection box 73 on the screen 70A at a timing when it is determined that the treatment by the user (operator) has ended, an input of the treatment name can be accepted without hindering the treatment work of the user (operator). The timing when the treatment name selection box 73 is displayed can be set to a timing when the treatment tool detection unit 63D has detected the treatment tool, a timing when a fixed time has elapsed after the treatment tool detection unit 63D has detected the treatment tool, and a timing when the end of the treatment name is determined by other image recognition. Further, the timing when the treatment name selection box 73 is displayed may be set according to the detected treatment tool.

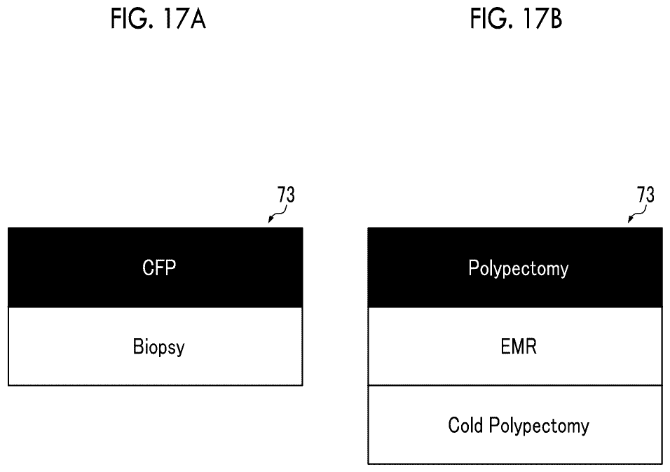

FIGS. 17A and 17B are diagrams illustrating examples of the treatment name selection box.

As illustrated in the figures, the treatment name selection box 73 is configured by a so-called list box, and selectable treatment names are displayed in a list. In the example illustrated in FIGS. 17A and 17B, a case in which a list of selectable treatment names is displayed in a vertical line is illustrated.

In the treatment name selection box 73, the name corresponding to the treatment tool 80 detected from the endoscopic image I is displayed. FIG. 17A illustrates an example of the treatment name selection box 73 displayed on the screen in a case where the treatment tool 80 detected from the endoscopic image I is the “biopsy forceps”. As illustrated in the figure, in a case where the detected treatment tool is the “biopsy forceps”, “cold forceps polypectomy (CFP)” and “Biopsy” are displayed as the selectable treatment names. FIG. 17B illustrates an example of the treatment name selection box 73 displayed on the screen in a case where the treatment tool 80 detected from the endoscopic image I is the “snare”. As illustrated in the figure, in a case where the detected treatment tool is the “snare”, “Polypectomy”, “endoscopic mucosal resection (EMR)”, and “Cold Polypectomy” are displayed as the selectable treatment names.

In FIGS. 17A and 17B, a treatment name displayed in white characters on a black background indicates the treatment name being selected. In the example illustrated in FIG. 17A, a case where “CFP” is selected is illustrated. Further, in the example illustrated in FIG. 17B, a case where “Polypectomy” is selected is illustrated.

In a case where the treatment name selection box 73 is displayed on the screen, the display control unit 64 displays the treatment name selection box 73 on the screen in a state where one treatment name is selected in advance. Further, in a case where the treatment name selection box 73 is displayed on the screen, the display control unit 64 displays the treatment names in a predetermined arrangement in the treatment name selection box 73. Therefore, the display control unit 64 controls the display of the treatment name selection box 73 by referring to the table.

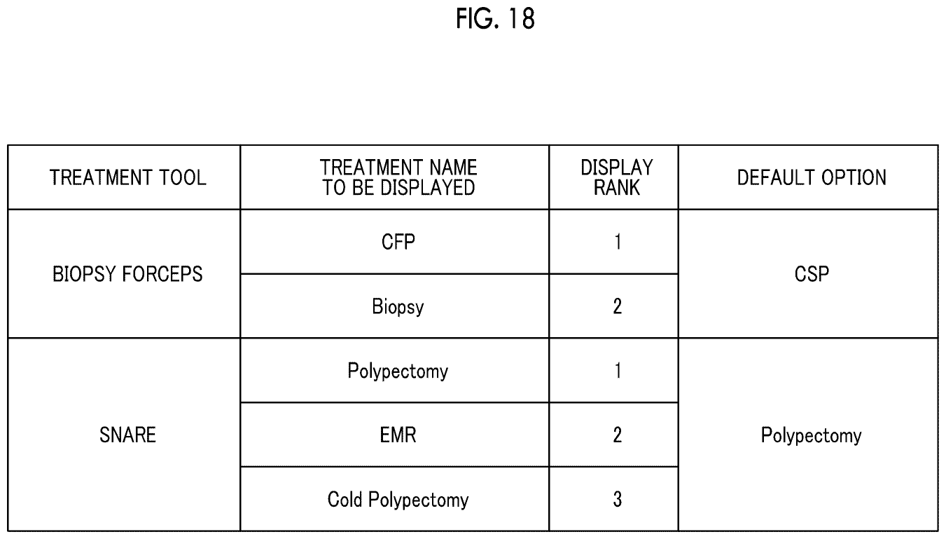

FIG. 18 is a diagram illustrating an example of the table.

As illustrated in the figure, in the table, pieces of information on “treatment tool”, “treatment name to be displayed”, “display rank”, and “default option” are registered in association with each other. Here, the “treatment tool” in the same table is the type of the treatment tool to be detected from the endoscopic image I. The “treatment name to be displayed” is the treatment name to be displayed corresponding to the treatment tool. The “display rank” is a display order of each treatment name to be displayed. In a case where the treatment names are displayed in a vertical line, the treatment names are ranked 1, 2, 3, and the like from the top. The “default option” is the treatment name that is first selected.

The “treatment name to be displayed” may not necessarily be the treatment names of all the treatments executable by the corresponding treatment tool. It is preferable to limit the number of treatment names to a smaller number. That is, it is preferable to limit the number to a specified number or less. In this case, in a case where the number of types of treatments executable by a certain treatment tool exceeds a specified number, the number of treatment names to be registered in the table (treatment names displayed in the treatment name selection box) is limited to a specified number or less.

In a case where the number of treatment names to be displayed is limited, a treatment name with a high execution frequency is chosen from among the treatment names of the executable treatments. For example, in a case where the “treatment tool” is the “snare”, (1) “Polypectomy”, (2) “EMR”, (3) “Cold Polypectomy”, (4) “EMR [en bloc]”, (5) “EMR [piecemeal: <5 pieces]”, (6) “EMR [piecemeal: ≥5 pieces]”, (7) “endoscopic submucosal resection with a ligation device (ESMR-L)”, (8) “endoscopic mucosal resection using a cap-fitted endoscope (EMR-C)”, and the like are exemplified as the treatment names of executable treatments. It is assumed that (1) “Polypectomy”, (2) “EMR”, (3) “Cold Polypectomy”, (4) “EMR [en bloc]”, (5) “EMR [piecemeal: <5 pieces]”, (6) “EMR [piecemeal: ≥5 pieces]”, (7) “ESMR-L”, and (8) “EMR-C” are arranged in the descending order of the execution frequency, and the specified number is three. In this case, three of (1) Polypectomy, (2) EMR, and (3) Cold Polypectomy are registered in the table as the “treatment name to be displayed”. Note that each of (4) “EMR [en bloc]”, (5) “EMR [piecemeal: <5 pieces]”, and (6) “EMR [piecemeal: ≥5 pieces]” is a treatment name in a case of inputting a detailed treatment name by EMR. (4) EMR [en bloc] is a treatment name in a case of the en bloc resection by EMR. (5) EMR [piecemeal: <5 pieces] is a treatment name in a case of the piecemeal resection by EMR with less than 5 pieces. (6) EMR [piecemeal: ≥5 pieces] is a treatment name in a case of the piecemeal resection by EMR with 5 pieces or more.

The specified number can be determined for each treatment tool. For example, the number (specified number) of treatment names to be displayed for each treatment tool can be determined such that the specified number is two for the “biopsy forceps” and the specified number is three for the “snare”. For the “biopsy forceps”, for example, “Hot Biopsy” is exemplified as the executable treatment in addition to the “CFP” and the “Biopsy”.

In this manner, by narrowing down the treatment names with a high execution frequency (treatment names having a high probability of being selected) and displaying the options (selectable treatment names) in the treatment name selection box 73, the user can efficiently select the treatment name. In a case where a plurality of treatments can be executed by the same treatment tool, the detection of the treatment (treatment name) executed by the treatment tool may be more difficult than the detection of the type of the treatment tool (image recognition). By associating the treatment name that may be executed with the treatment tool in advance and allowing the user to select the treatment name, it is possible to select an appropriate treatment name with a small number of operations.

The “display rank” is ranked 1, 2, 3, and the like in the descending order of the execution frequency. Normally, the higher the execution frequency is, the higher the selection frequency is, so the descending order of the execution frequency is synonymous with the descending order of the selection frequency.

In the “default option”, the treatment name with the highest execution frequency among the treatment names to be displayed is selected. The highest execution frequency is synonymous with the highest selection frequency.

In the example illustrated in FIG. 18, in a case where the “treatment tool” is the “biopsy forceps”, the “treatment name to be displayed” is “CFP” and “Biopsy”. Then, the “display rank” is in the order of “CFP” and “Biopsy” from the top, and the “default option” is “CFP” (refer to FIG. 17A).

Further, in a case where the “treatment tool” is the “snare”, the “treatment name to be displayed” is “Polypectomy”, “EMR”, and “Cold Polypectomy”. Then, the “display rank” is in the order of “Polypectomy”, “EMR”, and “Cold Polypectomy” from the top, and the “default option” is “Polypectomy” (refer to FIG. 17B).

The display control unit 64 chooses treatment names to be displayed in the treatment name selection box 73 by referring to the table on the basis of the information on the treatment tool detected by the treatment tool detection unit 63D. Then, the chosen treatment names are arranged according to the information on the display rank registered in the table, and the treatment name selection box 73 is displayed on the screen. The treatment name selection box 73 is displayed on the screen in a state where one treatment name is selected according to the information on the default option registered in the table. In this manner, by displaying the treatment name selection box 73 in a state where one treatment name is selected in advance, in a case where there is no need to change, it is possible to save time and effort for the selection, and to efficiently input the information on the treatment name. Further, by setting the treatment name selected in advance as the treatment name of the treatment with a high execution frequency (=treatment with a high selection frequency), it is possible to save time and effort for the change. Further, by arranging the treatment names to be displayed in the treatment name selection box 73 in the descending order of the execution frequency (=descending order of selection frequency), the user can efficiently select the treatment name. Moreover, by narrowing down and displaying the options, the user can efficiently select the treatment name. The display content and the display order of the treatment name can be set for each hospital (including examination facility) and for each device. Further, the default option may be set to the previously executed treatment name during the examination. Since the same treatment may be repeated during the examination, it is possible to save time and effort for the change by selecting the previous treatment name as the default.

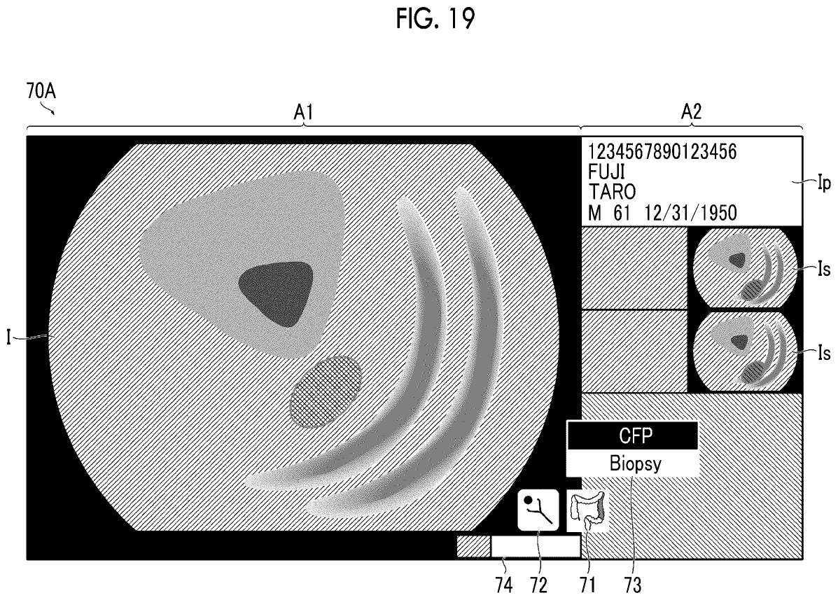

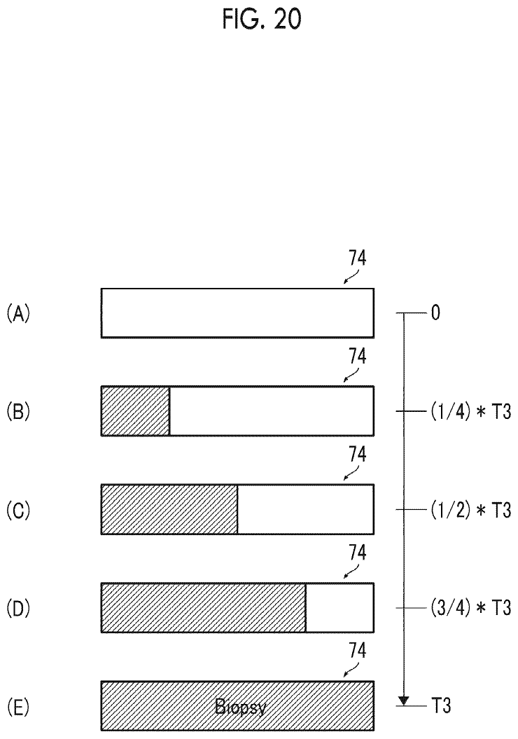

FIG. 19 is a diagram illustrating an example of the display position of the treatment name selection box. The treatment name selection box 73 is displayed at a fixed position on the screen 70A. More specifically, the treatment name selection box 73 is displayed as a pop-up at a fixed position. In the present embodiment, the treatment name selection box 73 is displayed in the vicinity of the treatment tool detection icon 72. More specifically, the treatment name selection box 73 is displayed adjacent to the treatment tool detection icon 72. In the example illustrated in FIG. 19, a case in which the treatment name selection box 73 is displayed adjacent to an upper right portion of the treatment tool detection icon 72 is illustrated. Since the treatment name selection box 73 is displayed adjacent to the treatment tool detection icon 72, the treatment name selection box 73 is displayed in the vicinity of the position where the treatment tool appears in the endoscopic image I. In this manner, by displaying the treatment name selection box 73 in the vicinity of the position where the treatment tool 80 appears in the endoscopic image I, it is easier for the user to recognize the presence of the treatment name selection box 73. That is, it is possible to improve visibility.

Note that FIG. 19 illustrates a display example in a case where the “biopsy forceps” are detected as the treatment tool. In this case, the treatment name selection box 73 corresponding to the “biopsy forceps” is displayed (refer to FIG. 17A). The region where the treatment name selection box 73 is displayed on the screen is an example of a third region.

The display control unit 64 displays the treatment name selection box 73 on the screen 70A for a fixed time (time T3). For example, time T3 is 15 seconds. Time T3 may be arbitrarily set by the user. Time T3 is an example of a second time. The display time for the treatment name selection box 73 may be decided according to the detected treatment tool. Further, the display time for the treatment name selection box 73 may be set by the user.

The user can select the treatment name while the treatment name selection box 73 is displayed on the screen. The selection method will be described later.

As described above, the treatment name selection box 73 is displayed on the screen in a state where one treatment name is selected in advance. The user performs selection processing in a case where the treatment name selected by default is different from the actual treatment name. For example, in a case where the treatment tool used is the “biopsy forceps”, the treatment name selection box 73 is displayed on the screen 70A in a state where “CFP” is selected, but in a case where the treatment actually performed is “Biopsy”, the user performs the selection processing.

In a case where a fixed time (time T3) has elapsed from the display start of the treatment name selection box 73, and the treatment name selection box 73 disappears from the screen 70A, the selection is confirmed. That is, in the endoscope system 10 of the present embodiment, the selection can be automatically confirmed without performing selection confirmation processing separately. Therefore, for example, in a case where the treatment name selected by default is correct, the treatment name can be input without performing any input operation. Accordingly, it is possible to greatly reduce time and effort for inputting the treatment name.

Since the time for which the selection operation of the treatment name is possible is limited, in the endoscope system 10 of the present embodiment, the remaining time until the acceptance of the selection is ended is displayed on the screen. In the present embodiment, the remaining time until the acceptance of the selection is ended is displayed by displaying a progress bar 74 at a fixed position on the screen. FIG. 20 is a diagram illustrating an example of the progress bar. The figure illustrates a temporal change of the display of the progress bar 74. (A) of FIG. 20 illustrates the display of the progress bar 74 in a case where the display of the treatment name selection box 73 is started. Further, (B) to (D) of FIG. 20 respectively illustrate the display of the progress bar 74 after a time of (1/4)*T3 has elapsed from the start of the display of the treatment name selection box 73, after a time of (2/4)*T3 has elapsed from the start of the display of the treatment name selection box 73, and after a time of (3/4)*T3 has elapsed from the start of the display of the treatment name selection box 73. Further, (E) of FIG. 20 illustrates the display of the progress bar 74 in a case where a time of T3 has elapsed from the start of the display of the treatment name selection box 73. That is, the display of the progress bar 74 in a case where the acceptance of the selection is ended is illustrated. As illustrated in the figure, in the progress bar 74 in this example, the remaining time is indicated by a bar in a horizontal direction in which the bar is filled from left to right. In this case, a white portion indicates the remaining time. The display of the remaining time can be displayed by a numerical value instead of the progress bar or in addition to the progress bar. That is, the remaining time can also be counted down and displayed in seconds.

As described above, in the endoscope system 10 of the present embodiment, the selection is automatically confirmed by the end of the acceptance of the selection of the treatment name. In a case where the acceptance of the selection of the treatment name is ended, the treatment name for which the selection is confirmed is displayed at the display position of the progress bar 74 as illustrated in (E) of FIG. 20. The user can check the treatment name selected by himself/herself by viewing the display of the progress bar 74. (E) of FIG. 20 illustrates an example of a case where “Biopsy” is selected.

As illustrated in FIG. 19, the progress bar 74 is displayed in the vicinity of the display position of the treatment tool detection icon 72. Specifically, the progress bar 74 is displayed adjacent to the treatment tool detection icon 72. In the example illustrated in FIG. 19, a case in which the progress bar 74 is displayed adjacent to a lower portion of the treatment tool detection icon 72 is illustrated. Since the progress bar 74 is displayed adjacent to the treatment tool detection icon 72, the progress bar 74 is displayed in the vicinity of the position where the treatment tool appears in the endoscopic image I. In this manner, by displaying the progress bar 74 in the vicinity of the position where the treatment tool 80 appears in the endoscopic image I, it is easier for the user to recognize the presence of the progress bar 74.

The time (time T3) for which the treatment name selection box 73 is displayed extends under certain conditions. Specifically, the time extends in a case where the selection processing of the treatment name is performed. The extension of the time is performed by resetting the countdown. Therefore, the time extends by the difference between time T3 and the remaining time at a time point when the selection processing is performed. For example, in a case where the remaining time at the time point when the selection processing is performed is ΔT, the display time extends by (T3−ΔT). In other words, the selection is possible again for time T3 from the time point when the selection processing is performed.

The extension of the display time is executed each time the selection processing is performed. That is, the countdown is reset each time the selection processing is performed, so that the display time extends. Further, accordingly, the period for the acceptance of the selection of the treatment name extends.

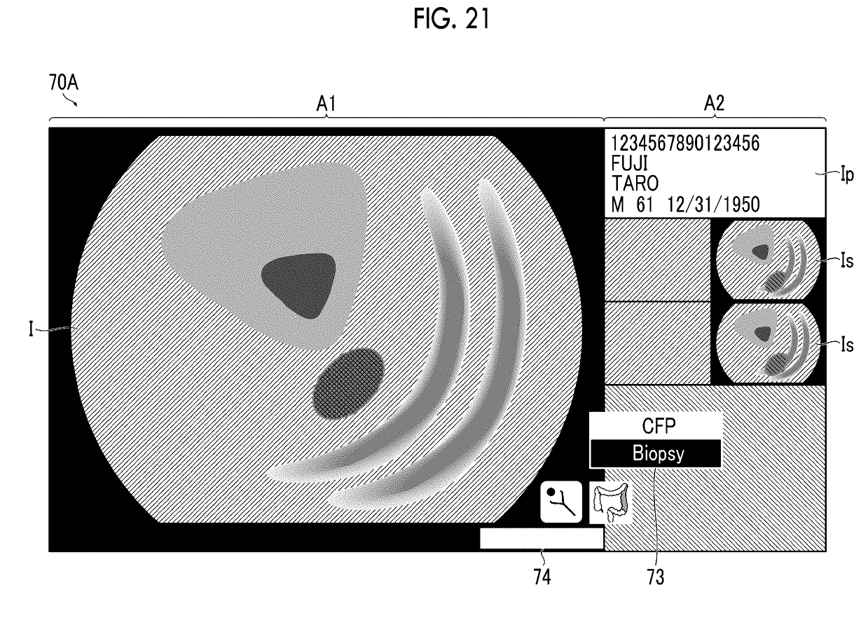

FIG. 21 is a diagram illustrating an example of the display of the screen immediately after the selection processing of the treatment name is performed.

As illustrated in the figure, in a case where the user performs the selection processing of the treatment name, the display of the progress bar 74 is reset.

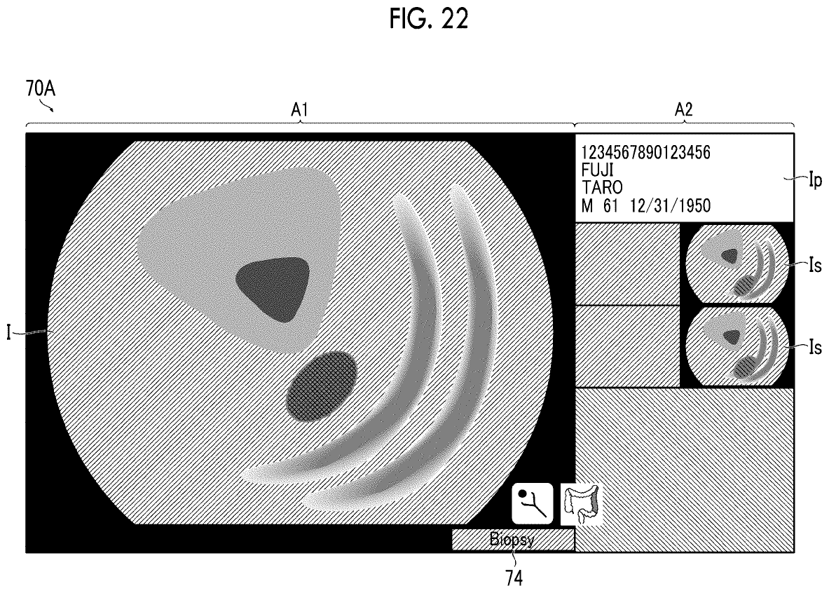

FIG. 22 is a diagram illustrating an example of the display of the screen immediately after the acceptance of the selection of the treatment name is ended.

As illustrated in the figure, in a case where the acceptance of the selection of the treatment name is ended, the display of the treatment name selection box 73 disappears. Meanwhile, the treatment name for which the selection is confirmed is displayed in the progress bar 74. FIG. 22 illustrates an example of a case where “Biopsy” is selected.

Information on the treatment name for which the selection is confirmed is displayed at the display position of the progress bar 74 for a fixed time (time T4). Then, after a fixed time has elapsed, the display disappears. In this case, the display of the treatment tool detection icon 72 also disappears.

Here, a method of selecting a site in a case where the site selection box 71 is displayed, and a method of selecting a treatment name in a case where the treatment name selection box 73 is displayed will be described.

The selection of the site and the selection of the treatment name are both performed using the input device 50. In particular, in the present embodiment, the selection is performed using the foot switch constituting the input device 50. Each time the foot switch is stepped on, the operation signal is output.

First, the method of selecting a site will be described. In principle, the selection of the site is always accepted after the start of the display of the site selection box 71 until the examination ends. As an exception, the acceptance of the selection of the site is stopped while the selection of the treatment name is being accepted. That is, the acceptance of the selection of the site is stopped while the treatment name selection box 73 is being displayed. The time for which the selection of the treatment name is accepted (=time for which the acceptance of the selection of the site is stopped) is an example of the second time and the third time.

In a case where the foot switch is operated in a state where the selection of the site is being accepted, the site being selected is switched in order. In the present embodiment, (1) ascending colon, (2) transverse colon, and (3) descending colon are looped and switched in this order. Therefore, for example, in a case where the foot switch is operated once in a state where the “ascending colon” is being selected, the selected site is switched from the “ascending colon” to the “transverse colon”. Similarly, in a case where the foot switch is operated once in a state where the “transverse colon” is being selected, the selected site is switched from the “transverse colon” to the “descending colon”. Moreover, in a case where the foot switch is operated once in a state where the “descending colon” is being selected, the selected site is switched from the “descending colon” to the “ascending colon”. In this manner, the selected site is switched in order each time the foot switch is operated once. The information on the selected site is stored in the main storage unit or the auxiliary storage unit. The information on the selected site can be used as information for specifying the site under observation. For example, in a case where the static image is captured during the examination, the site where the static image is captured can be specified after the examination by recording (storing) the captured static image and the information on the site being selected in association with each other. The information on the site being selected may be recorded in association with the time information during the examination or the elapsed time from the examination start. Accordingly, for example, in a case where the image captured by the endoscope is recorded as a video, the site can be specified from the time point or elapsed time. Further, the information on the site being selected may be recorded in association with the information on a lesion or the like detected by the image recognition processing unit 63. For example, in a case where the lesion part or the like is detected, the information on the lesion part or the like can be recorded in association with the information on the site being selected in a case where the lesion part or the like is detected.

Next, the method of selecting the treatment name will be described. As described above, the selection of the treatment name is accepted only while the treatment name selection box 73 is displayed. Similar to the case of the selection of the site, in a case where the foot switch is operated, the treatment name being selected is switched in order. The switching is performed according to the display rank. Therefore, the treatment names are switched in order from the top. Further, the treatment names are looped and switched. For example, in a case of the treatment name selection box 73 illustrated in FIG. 17A, a selection target is alternately switched between “CFP” and “Biopsy” each time the foot switch is operated once. That is, in a case where the foot switch is operated once in a state where “CFP” is being selected, the selection target is switched to “Biopsy”, and in a case where the foot switch is operated once in a state where “Biopsy” is being selected, the selection target is switched to “CFP”. Further, for example, in a case of the treatment name selection box 73 illustrated in FIG. 17B, the selection target is looped and switched in the order of (1) “Polypectomy”, (2) “EMR”, and (3) “Cold Polypectomy” each time the foot switch is operated once. Specifically, in a case where the foot switch is operated once in a state where the “Polypectomy” is being selected, the selection target is switched to “EMR”. Further, in a case where the foot switch is operated once in a state where “EMR” is being selected, the selection target is switched to “Cold Polypectomy”. Further, in a case where the foot switch is operated once in a state where the “Cold Polypectomy” is being selected, the selection target is switched to “Polypectomy”. The information on the selected treatment name is recorded together with the information on the detected treatment tool in the main storage unit or the auxiliary storage unit in association with the information on the site being selected.

[Examination Information Output Control Unit]

The examination information output control unit 65 outputs the examination information to the endoscope information management system 100. In the examination information, the endoscopic image captured during the examination, the information on the site input during the examination, the information on the treatment name input during the examination, the information on the treatment tool detected during the examination, and the like are included. For example, the examination information is output for each lesion or each time a specimen is collected. In this case, respective pieces of information are output in association with each other. For example, the endoscopic image in which the lesion part or the like is imaged is output in association with the information on the site being selected. Further, in a case where the treatment is performed, the information on the selected treatment name and the information on the detected treatment tool are output in association with the endoscopic image and the information on the site. Further, the endoscopic image captured separately from the lesion part or the like is always output to the endoscope information management system 100. The endoscopic image is output with the information of imaging date and time added.

[Display Device]

The display device 70 is an example of a display unit. For example, the display device 70 includes a liquid-crystal display (LCD), an organic electroluminescence (EL) display (OLED), or the like. In addition, the display device 70 includes a projector, a head-mounted display, and the like. The display device 70 is an example of a first display unit.

[Endoscope Information Management System]

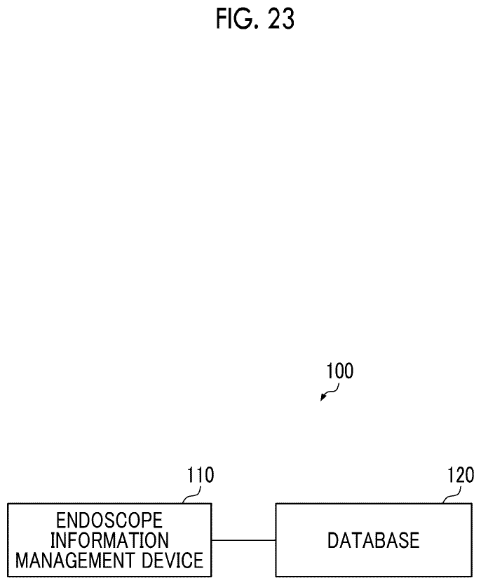

FIG. 23 is a block diagram illustrating an example of a system configuration of the endoscope information management system.

As illustrated in the figure, the endoscope information management system 100 mainly includes an endoscope information management device 110 and a database 120.

The endoscope information management device 110 collects a series of information (examination information) related to the endoscopy, and integrally manages the series of information. Further, the creation of an examination report is supported via the user terminal 200.

The endoscope information management device 110 includes, as a hardware configuration, a processor, a main storage unit, an auxiliary storage unit, a display unit, an operation unit, a communication unit, and the like. That is, the endoscope information management device 110 has a so-called computer configuration as the hardware configuration. For example, the processor is configured by a CPU. The processor of the endoscope information management device 110 is an example of a second processor. For example, the main storage unit is configured by a RAM. For example, the auxiliary storage unit is configured by a hard disk drive (HDD), a solid-state drive (SSD), a flash memory, or the like. The display unit is configured by a liquid-crystal display, an organic EL display, or the like. The operation unit is configured by a keyboard, a mouse, a touch panel, or the like. For example, the communication unit is configured by a communication interface connectable to a network. The endoscope information management device 110 is communicably connected to the endoscope system 10 via the communication unit. More specifically, the endoscope information management device 110 is communicably connected to the endoscopic image processing device 60.

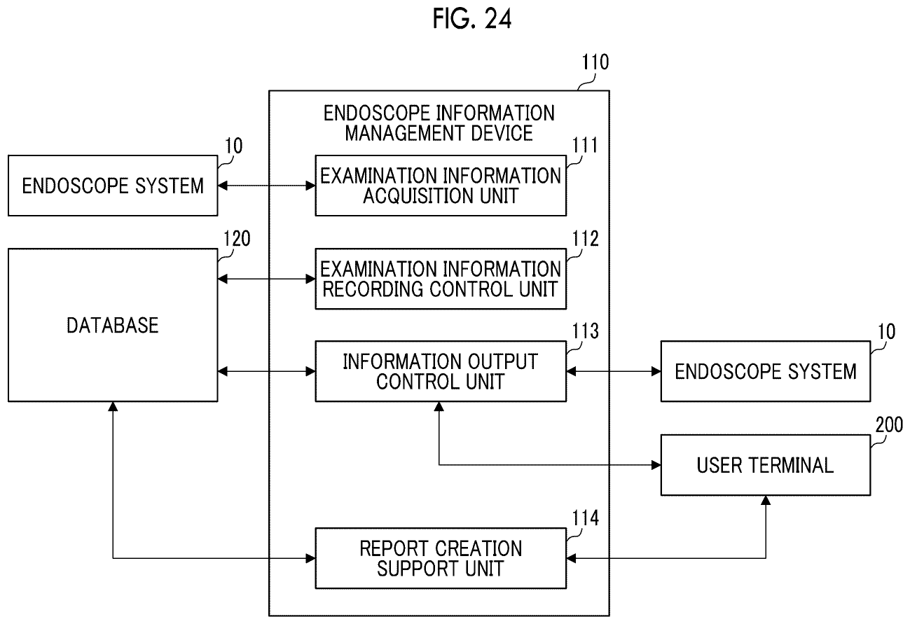

FIG. 24 is a block diagram of main functions of the endoscope information management device.

As illustrated in the figure, the endoscope information management device 110 has functions of an examination information acquisition unit 111, an examination information recording control unit 112, an information output control unit 113, a report creation support unit 114, and the like. Each function is realized by the processor executing a predetermined program. The auxiliary storage unit stores various programs executed by the processor, various kinds of data necessary for processing, and the like.

The examination information acquisition unit 111 acquires the series of information (examination information) related to the endoscopy from the endoscope system 10. In the information to be acquired, the endoscopic image captured during the examination, the information on the site input during the examination, the information on the treatment name, the information on the treatment tool, and the like are included. In the endoscopic image, a video and a static image are included.

The examination information recording control unit 112 records the examination information acquired from the endoscope system 10 in the database 120.

The information output control unit 113 controls the output of the information recorded in the database 120. For example, the information recorded in the database 120 is output to a request source in response to a request from the user terminal 200, the endoscope system 10, and the like.

The report creation support unit 114 supports the creation of the report on the endoscopy via the user terminal 200. Specifically, a report creation screen is provided to the user terminal 200 to support the input on the screen.

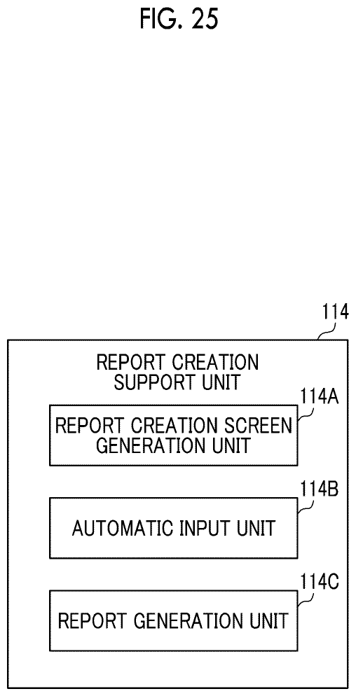

FIG. 25 is a block diagram of main functions of the report creation support unit.

As illustrated in the figure, the report creation support unit 114 has functions of a report creation screen generation unit 114A, an automatic input unit 114B, a report generation unit 114C, and the like.

In response to the request from the user terminal 200, the report creation screen generation unit 114A generates a screen necessary for creating a report (report creation screen), and provides the screen to the user terminal 200.

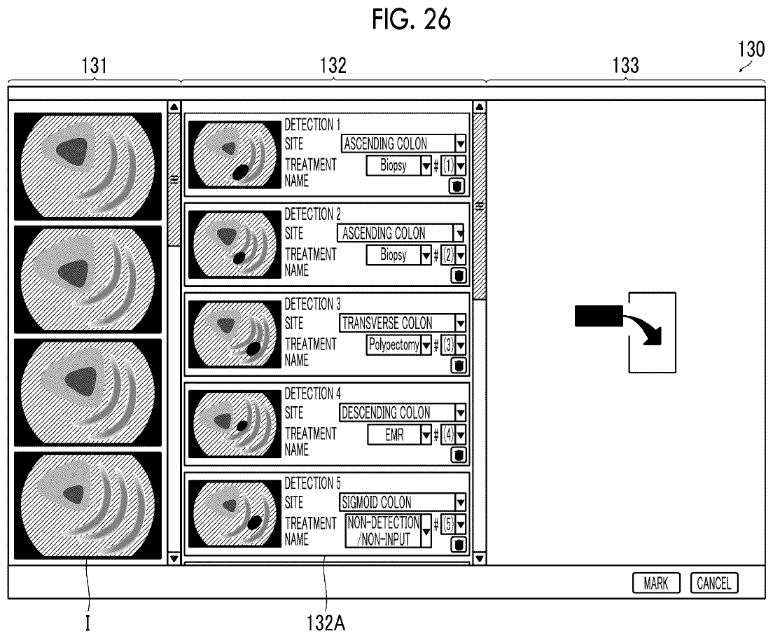

FIG. 26 is a diagram illustrating an example of a selection screen.

A selection screen 130 is one of the report creation screens, and is a screen for selecting a report creation target or the like. As illustrated in the figure, the selection screen 130 has a captured image display region 131, a detection list display region 132, a merge processing region 133, and the like.

The captured image display region 131 is a region in which the static images Is captured during the examination in one endoscopy are displayed. The captured static images Is are displayed in chronological order.

The detection list display region 132 is a region in which the detected lesion or the like is displayed in a list. The detected lesion or the like is displayed by a card 132A in a list in the detection list display region 132. On the card 132A, in addition to the endoscopic image in which the lesion or the like is imaged, the information on the site, the information on the treatment name (information on a specimen collection method in a case of specimen collection), and the like are displayed. The information on the site, the information on the treatment name, and the like can be corrected on the card. In the example illustrated in FIG. 26, by pressing a drop-down button provided in a display column of each piece of information, a drop-down list is displayed, and the information can be corrected. The card 132A is displayed in a detection order from top to bottom in the detection list display region 132.

The merge processing region 133 is a region in which merge processing is performed on the card 132A. The merge processing is performed by dragging the card 132A to be merged to the merge processing region 133.

On the selection screen 130, the user designates the card 132A displayed in the detection list display region 132, and selects the lesion or the like as the report creation target.

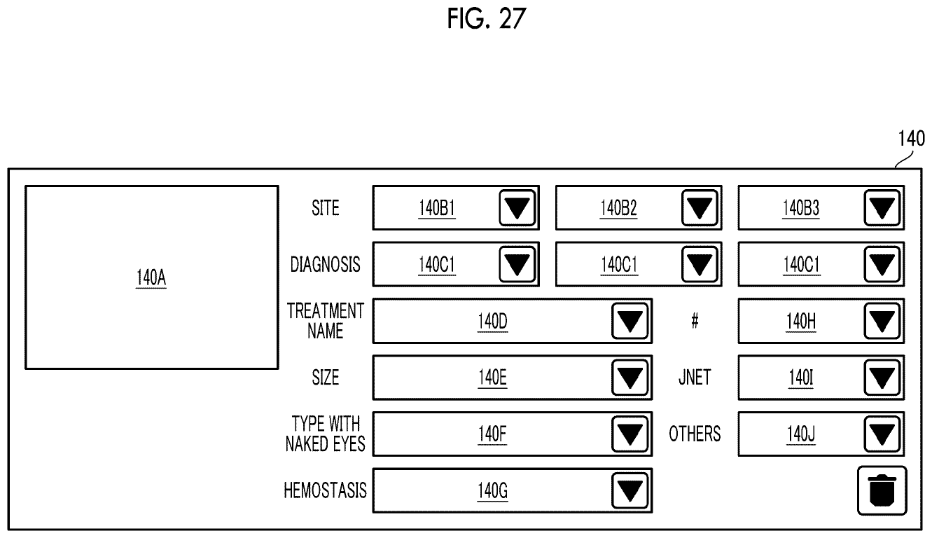

FIG. 27 is a diagram illustrating an example of a detailed input screen.

A detailed input screen 140 is one of the report creation screens, and is a screen for inputting various kinds of information necessary for generating a report. As illustrated in the figure, the detailed input screen 140 has a plurality of input fields 140A to 140J for inputting various kinds of information necessary for generating a report.

The input field 140A is an input field for an endoscopic image (static image). The endoscopic image (static image) to be attached to the report is input to the input field 140A.

The input fields 140B1 to 140B3 are input fields for information on a site. A plurality of input fields are prepared for the site so that the information thereof can be input hierarchically. In the example illustrated in FIG. 27, three input fields are prepared such that information on the site can be input in three hierarchies. The input is performed by selecting one option from the drop-down list. The drop-down list is displayed by pressing (clicking or touching) the drop-down button provided in each of the input fields 140B1 to 140B3.

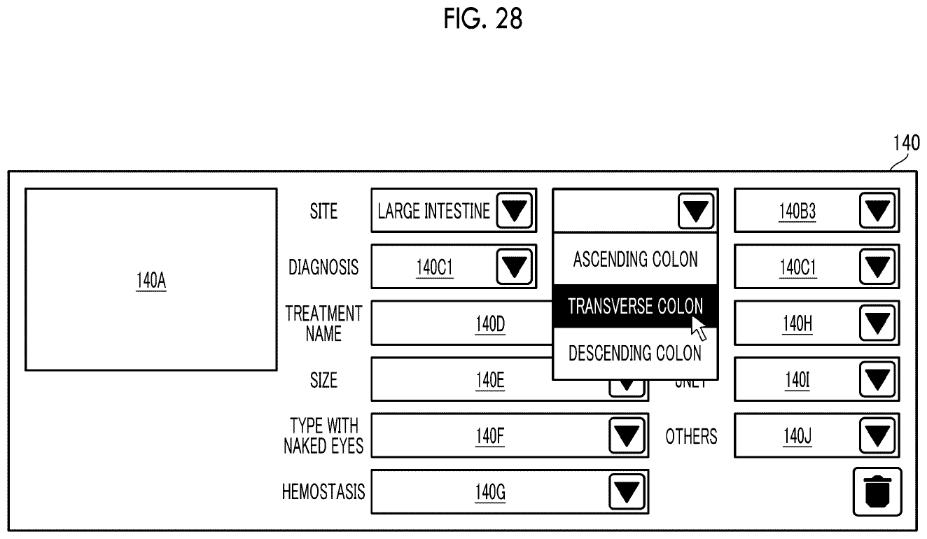

FIG. 28 is a diagram illustrating an example of the display of the drop-down list. FIG. 28 illustrates an example of the drop-down list displayed in the input field 140B2 of the second hierarchy for a site.

As illustrated in the figure, in the drop-down list, options are displayed in a list for the designated input field. The user selects one option from among the options displayed in a list, and inputs the one option in a target input field. In the example illustrated in the figure, a case where there are three options of “ascending colon”, “transverse colon”, and “descending colon” is illustrated.

The input fields 140C1 to 140C3 are input fields for information on the diagnosis result. Similarly, a plurality of input fields are prepared for the diagnosis result so that the information thereof can be input hierarchically. In the example illustrated in FIG. 28, three input fields are prepared such that information on the diagnosis result can be input in three hierarchies. The input is performed by selecting one option from the drop-down list. The drop-down list is displayed by pressing the drop-down button provided in each of the input fields 140C1 to 140C3. Selectable diagnosis names are displayed in a list in the drop-down list.

The input field 140D is an input field for information on the treatment name. The input is performed by selecting one option from the drop-down list. The drop-down list is displayed by pressing the drop-down button provided in the input field 140D. Selectable treatment names are displayed in a list in the drop-down list.

The input field 140E is an input field for information on the size of the lesion or the like. The input is performed by selecting one option from the drop-down list. The drop-down list is displayed by pressing the drop-down button provided in the input field 140E. Selectable numerical values are displayed in a list in the drop-down list.

The input field 140F is an input field for information on classification by a naked eye. The input is performed by selecting one option from the drop-down list. The drop-down list is displayed by pressing the drop-down button provided in the input field 140F. Selectable classifications are displayed in a list in the drop-down list.

The input field 140G is an input field for information on hemostatic methods. The input is performed by selecting one option from the drop-down list. The drop-down list is displayed by pressing the drop-down button provided in the input field 140G. Selectable hemostatic methods are displayed in a list in the drop-down list.

The input field 140H is an input field for information on specimen number. The input is performed by selecting one option from the drop-down list. The drop-down list is displayed by pressing the drop-down button provided in the input field 140H. Selectable numerical values are displayed in a list in the drop-down list.

The input field 140I is an input field for information on Japan NBI Expert Team (JNET) classification. The input is performed by selecting one option from the drop-down list. The drop-down list is displayed by pressing the drop-down button provided in the input field 140I. Selectable classifications are displayed in a list in the drop-down list.

The input field 140J is an input field for other information. The input is performed by selecting one option from the drop-down list. The drop-down list is displayed by pressing the drop-down button provided in the input field 140J. Pieces of information that can be input are displayed in a list in the drop-down list.

The automatic input unit 114B automatically inputs the information of the predetermined input fields of the detailed input screen 140 on the basis of the information recorded in the database 120. As described above, in the endoscope system 10 of the present embodiment, the information on the site and the information on the treatment name are input during the examination. The input information is recorded in the database 120. Thus, the information on the site and on the treatment name can be automatically input. The automatic input unit 114B acquires the information on the site and the information on the treatment name for the lesion or the like as the report creation target, from the database 120, and automatically inputs the information to the input fields 140B1 to 140B3 for the site and to the input field 140D for the treatment name of the detailed input screen 140. Further, the endoscopic image (static image) captured for the lesion or the like as the report creation target is acquired from the database 120, and is automatically input to the input field 140A for the image.

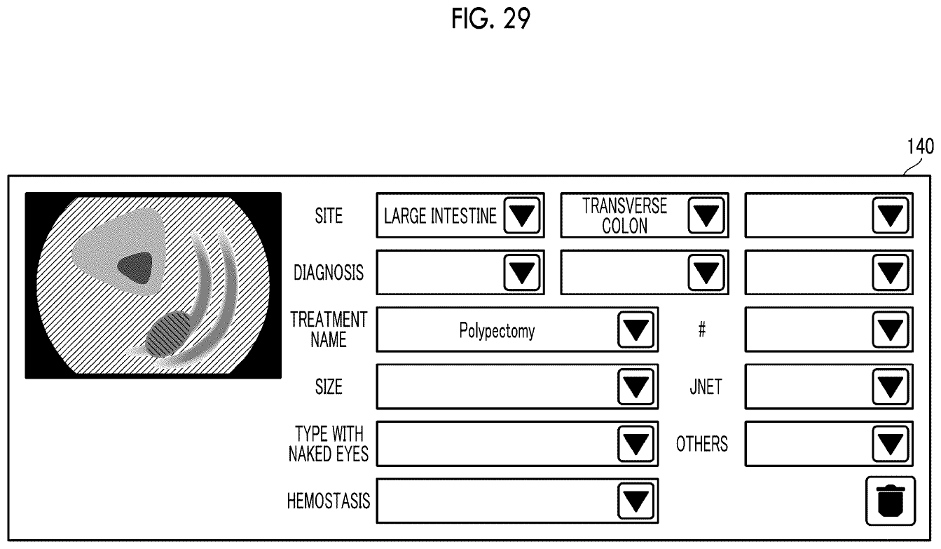

FIG. 29 is a diagram illustrating an example of the detailed input screen which is automatically filled.

As illustrated in the figure, the input field for the endoscopic image, the input field for the information on the site, and the input field for the information on the treatment name are automatically filled. As an initial screen of the detailed input screen 140, a screen in which the input field for the endoscopic image, the input field for the information on the site, and the input field for the information on the treatment name are automatically filled is provided to the user terminal 200. The user corrects the input field that is automatically filled, as necessary. For other input fields, in a case where the information to be input can be acquired, it is preferable to automatically input the information.

For example, correcting the input field for the endoscopic image is performed by dragging a target thumbnail image to the input field 140A from a thumbnail list of endoscopic images opened in a separate window.

Correcting the input field for the information on the site and the input field for the information on the treatment name is performed by selecting one option from the drop-down list.

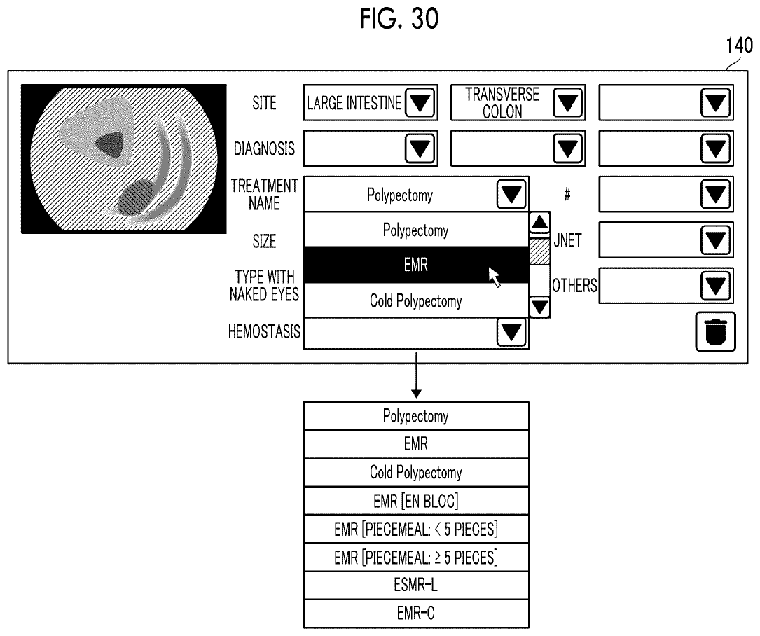

FIG. 30 is a diagram illustrating an example of the detailed input screen during correction. FIG. 30 illustrates an example of a case where the information of the input field for the treatment name is corrected.

As illustrated in the figure, the correction of the information is performed by selecting one option from the options displayed in the drop-down list.

Here, it is preferable that the number of options displayed in the drop-down list is set to be larger than the number of options displayed during the examination. For example, in a case where the treatment tool is the snare, the options of the treatment name displayed during the examination are three of “Polypectomy”, “EMR”, and “Cold Polypectomy”, as illustrated in FIG. 17B. On the other hand, the treatment names selectable in the detailed input screen 140 are eight of “Polypectomy”, “EMR”, “Cold Polypectomy”, “EMR [en bloc]”, “EMR [piecemeal: <5 pieces]”, “EMR [piecemeal: ≥5 pieces]”, “ESMR-L”, and “EMR-C”, as illustrated in FIG. 30. In this manner, in a case of creating a report, it is possible to easily correct target information by presenting more options. Meanwhile, during the examination, by narrowing down the options, it is possible for the user to efficiently select the treatment name.

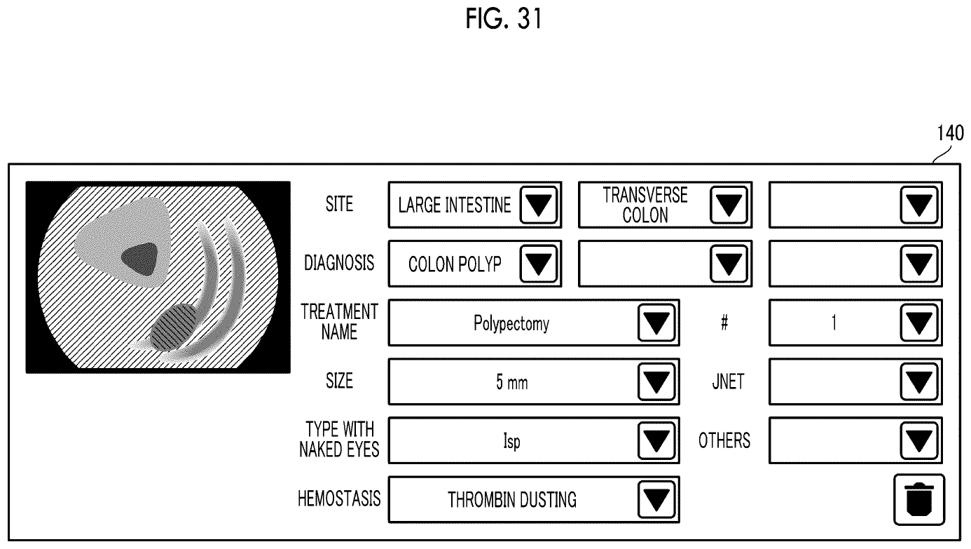

FIG. 31 is a diagram illustrating an example of the detailed input screen after the input is completed. As illustrated in the figure, the information to be entered in the report is input to each input field.

The report generation unit 114C automatically generates a report in a predetermined format, for the lesion or the like selected as the report creation target, on the basis of the information input on the detailed input screen 140. The generated report is presented on the user terminal 200.

[User Terminal]

The user terminal 200 is used for viewing various kinds of information related to the endoscopy, creating a report, and the like. The user terminal 200 includes, as a hardware configuration, a processor, a main storage unit, an auxiliary storage unit, a display unit, an operation unit, a communication unit, and the like. That is, the user terminal 200 has a so-called computer (for example, personal computer, tablet computer, or the like) configuration as the hardware configuration. For example, the processor is configured by a CPU. For example, the main storage unit is configured by a RAM. For example, the auxiliary storage unit is configured by a hard disk drive, a solid-state drive, a flash memory, or the like. The display unit is configured by a liquid-crystal display, an organic EL display, or the like. The operation unit is configured by a keyboard, a mouse, a touch panel, or the like. For example, the communication unit is configured by a communication interface connectable to a network. The user terminal 200 is communicably connected to the endoscope information management system 100 via the communication unit. More specifically, the user terminal 200 is communicably connected to the endoscope information management device 110.

In the endoscopic image diagnosis support system 1 of the present embodiment, the user terminal 200 constitutes the report creation support device together with the endoscope information management system 100. The display unit of the user terminal 200 is an example of a second display unit.

[Operation of Endoscopic Image Diagnosis Support System]

[Operation of Endoscope System During Examination]

In the following, the operation (information processing method) of the endoscope system 10 during the examination will be described focusing on an input operation of a site and an input operation of a treatment name during the examination.

[Input Operation of Site]

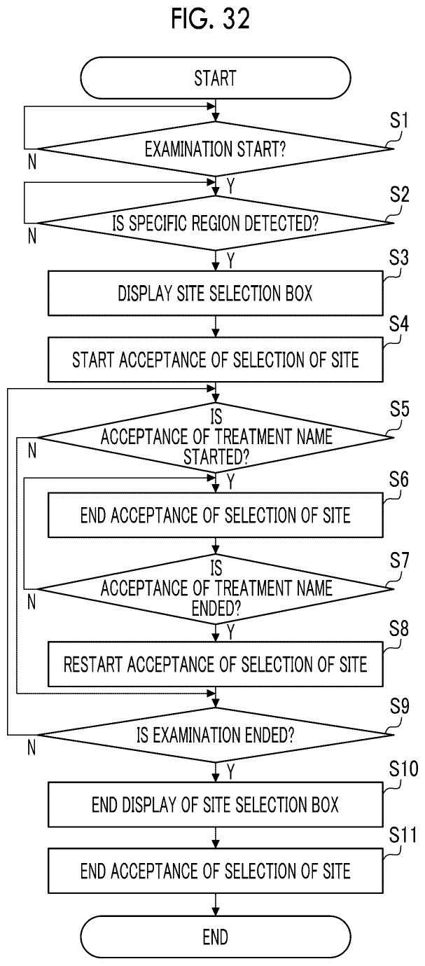

FIG. 32 is a flowchart illustrating a procedure of processing of accepting an input of a site.

First, it is determined whether or not the examination has started (Step S1). In a case where the examination has started, it is determined whether or not a specific region is detected from an image (endoscopic image) captured by the endoscope (Step S2). In the present embodiment, it is determined whether or not the ileocecum is detected as the specific region.

In a case where the specific region is detected from the endoscopic image, the site selection box 71 is displayed on the screen 70A of the display device 70 where the endoscopic image is being displayed (refer to FIG. 14) (Step S3). Further, the acceptance of the selection of the site is started (Step S4).

Here, the site selection box 71 is displayed in a state where a specific site is automatically selected in advance. Specifically, the site selection box 71 is displayed in a state where the site to which the specific region belongs is selected. In the present embodiment, the site selection box 71 is displayed in a state where the ascending colon is selected. In this manner, by displaying the site selection box 71 in a state where the site to which the specific region belongs is selected, it is possible to omit the user's initial selection operation. Accordingly, it is possible to efficiently input the information on the site. Further, accordingly, the user can concentrate on the examination.

In a case of starting the display, the site selection box 71 is displayed in an emphasized manner for a fixed time (time T1). In the present embodiment, as illustrated in FIG. 14, the site selection box 71 is displayed by being enlarged. In this manner, by displaying the site selection box 71 in an emphasized manner in a case of starting the display, it is easier for the user to recognize that the acceptance of the selection of the site is started. Further, it is easier for the user to recognize the site being selected.

In a case where a fixed time has elapsed from the start of the display, the site selection box 71 is displayed in a normal display state (refer to FIG. 13). Note that the acceptance of the selection is continued even in the normal display state.

Here, the selection of the site is performed by the foot switch. Specifically, the site being selected is switched in order each time the user operates the foot switch. Then, the display of the site selection box 71 is also switched according to the switching operation. That is, the display of the site being selected is switched.

Further, in a case where the selection operation of the site is performed, the site selection box 71 is displayed in an emphasized manner for a fixed time (time T1).

The information on the selected site is recorded in the main storage unit or the auxiliary storage unit. Therefore, in the initial state, the ascending colon is recorded as the information on the site being selected.

In a case where the site selection box 71 is displayed on the screen, and the acceptance of the selection of the site is started, it is determined whether or not the acceptance of the treatment name is started (Step S5).

In a case where it is determined that the acceptance of the selection of the treatment name is started, the acceptance of the selection of the site is stopped (Step S6). Note that the display of the site selection box 71 is continued. After that, it is determined whether or not the acceptance of the selection of the treatment name is ended (Step S7). In a case where it is determined that the acceptance of the selection of the treatment name is ended, the acceptance of the selection of the site is restarted (Step S8).

In a case where the acceptance of the selection of the site is restarted, it is determined whether or not the examination has ended (Step S9). Also in a case where it is determined in Step S5 that the acceptance of the treatment name is not started, it is determined whether or not the examination has ended (Step S9). The end of the examination is performed by the user inputting an instruction to end the examination. In addition, for example, the end of the examination can be detected from the image by using the AI or the trained model. For example, the end of the examination can be detected by detecting from the image that the distal end of the insertion part of the endoscope is pulled out of the body. Further, for example, the end of the examination can be detected by detecting an anus from the image.

In a case where it is determined that the examination has ended, the display of the site selection box 71 is ended (Step S10). That is, the display of the site selection box 71 disappears from the screen. Further, the acceptance of the selection of the site is ended (Step S11). Accordingly, the processing of accepting the input of the site is ended.

On the other hand, in a case where it is determined that the examination has not ended, the processing returns to Step S5, and processing of Step S5 and subsequent steps is executed again.

As described above, in the endoscope system 10 of the present embodiment, in a case where the specific region is detected from the endoscopic image, the site selection box 71 is displayed on the screen 70A, and the selection of the site is possible. The site selection box 71 is displayed on the screen 70A in a state where the site to which the specific region belongs is selected in advance. Accordingly, it is possible to omit the user's initial selection operation.

In principle, in a case where the site selection box 71 is displayed, the acceptance of the selection of the site is continued until the examination ends. However, in a case where the acceptance of the selection of the treatment name is started during the acceptance of the selection of the site, the acceptance of the site is stopped. Accordingly, it is possible to prevent the confliction of the input operations. The stopped acceptance of the selection of the site is restarted in a case where the acceptance of the selection of the treatment name is ended.

[Input Operation of Treatment Name]

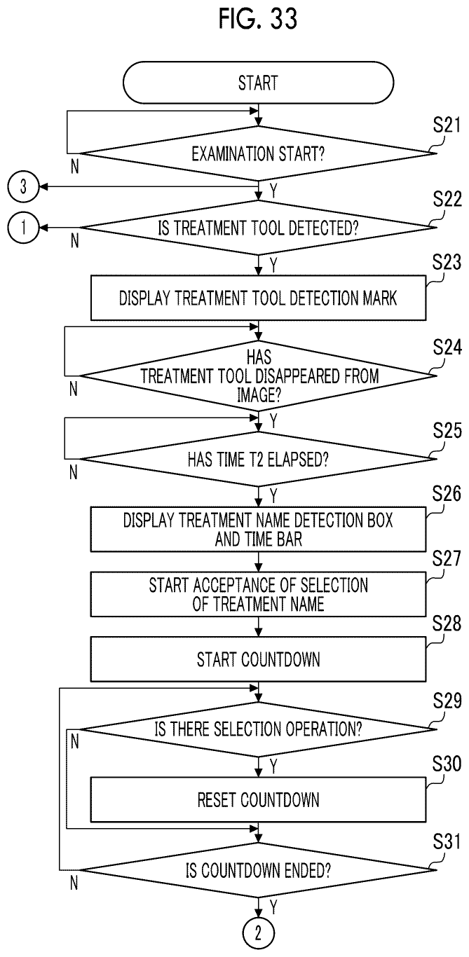

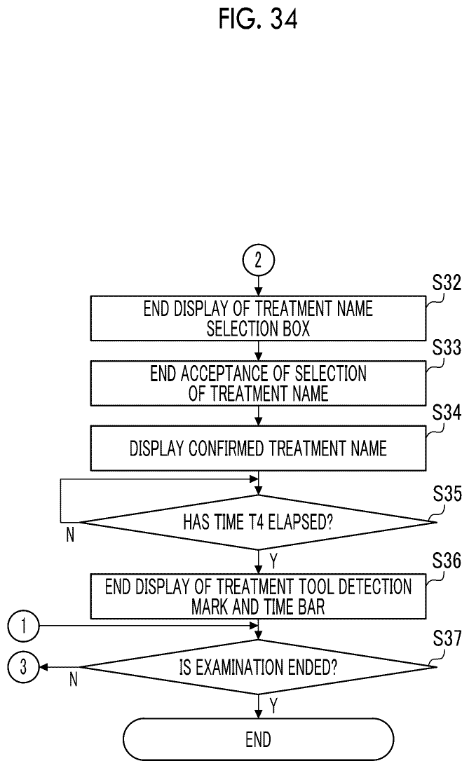

FIGS. 33 and 34 are flowcharts illustrating a procedure of processing of accepting an input of the treatment name.

First, it is determined whether or not the examination has started (Step S21). In a case where the examination has started, it is determined whether or not a treatment tool is detected from an image (endoscopic image) captured by the endoscope (Step S22).

In a case where the treatment tool is detected, the treatment tool detection icon 72 is displayed on the screen 70A of the display device 70 where the endoscopic image is being displayed (refer to FIG. 16) (Step S23). Thereafter, it is determined whether or not the treatment tool has disappeared from the endoscopic image (Step S24).

In a case where it is determined that the treatment tool has disappeared from the endoscopic image, it is determined whether or not a fixed time (time T2) has elapsed from the disappearance of the treatment tool (Step S25). In a case where a fixed time has elapsed from the disappearance of the treatment tool, the treatment is considered to have ended, and thus, the treatment name selection box 73 is displayed on the screen 70A of the display device 70. Further, at the same time, the progress bar 74 is displayed on the screen 70A of the display device 70 (refer to FIG. 19) (Step S26). In the treatment name selection box 73, the name corresponding to the detected treatment tool is displayed. For example, in a case where the detected treatment tool is the biopsy forceps, the treatment name selection box 73 for biopsy forceps is displayed (refer to FIG. 17A). Further, for example, in a case where the detected treatment tool is the snare, the treatment name selection box 73 for snare is displayed (refer to FIG. 17B). Further, the treatment names as the options to be displayed in the treatment name selection box 73 are displayed in a predetermined arrangement. Moreover, the treatment name selection box 73 is displayed in a state where one treatment name is automatically selected in advance. In this manner, by displaying the treatment name selection box 73 in a state where one treatment name is automatically selected in advance, it is possible to omit the user's initial selection operation in a case where the automatically selected treatment name is correct. Accordingly, it is possible to efficiently input the treatment name. Further, accordingly, the user can concentrate on the examination. The automatically selected treatment name is a treatment name with high execution frequency (treatment name with high selection frequency).

In a case where the treatment name selection box 73 is displayed on the screen 70A, the acceptance of the selection of the treatment name is started (Step S27). Further, the countdown of the display of the treatment name selection box 73 is started (Step S28).

As described above, in a case where the acceptance of the selection of the treatment name is started, the acceptance of the selection of the site is stopped. The acceptance of the selection of the site is stopped until the acceptance of the selection of the treatment name is ended.

In a case where the acceptance of the selection of the treatment name is started, it is determined whether or not there is a selection operation (Step S29). Here, the selection of the treatment name is performed by the foot switch. Specifically, the treatment name being selected is switched in order each time the user operates the foot switch. Then, the display of the treatment name selection box 73 is also switched according to the switching operation. That is, the display of the treatment name being selected is switched.

In a case where the selection operation of the treatment name is performed, the countdown of the display of the treatment name selection box 73 is reset (Step S30). Accordingly, the time for which the selection operation can be performed extends.

After that, it is determined whether or not the countdown has ended (Step S31). Also in a case where it is determined in Step S29 that there is no selection operation, it is determined whether or not the countdown has ended (Step S31).

In a case where the countdown has ended, the selected treatment name is confirmed. In a case where the user does not perform the selection operation of the treatment name during the countdown, the treatment name selected by default is confirmed. In this manner, since the treatment name is confirmed when the countdown has ended, it is possible to eliminate the need for a separate confirmation operation. Accordingly, it is possible to efficiently input information on the treatment name. Further, accordingly, the user can concentrate on the examination.

In a case where it is determined that the countdown has ended, the display of the treatment name selection box 73 is ended (Step S32). That is, the display of the treatment name selection box 73 disappears from the screen. Further, the acceptance of the selection of the treatment name is ended (Step S33).

Meanwhile, when the countdown has ended, the information on the confirmed treatment name is displayed at the display position of the progress bar 74 (refer to FIG. 22) (Step S34). The information on the confirmed treatment name is continuously displayed on the screen 70A for a fixed time (time T4). Therefore, in a case where the information on the confirmed treatment name is displayed at the display position of the progress bar 74, it is determined whether or not time T4 has elapsed from the start of the display (Step S35). In a case where it is determined that time T4 has elapsed, the display of the treatment tool detection icon 72 and of the progress bar 74 is ended (Step S36). That is, the display of the treatment tool detection icon 72 and of the progress bar 74 disappears from the screen 70A. The information on the confirmed treatment name also disappears with the disappearance of the display of the progress bar 74.

After that, it is determined whether or not the examination has ended (Step S37). The processing of accepting the input of the treatment name is ended by the end of the examination.

On the other hand, in a case where it is determined that the examination has not ended, the processing returns to Step S22, and processing of Step S22 and subsequent steps is executed again.

As described above, in the endoscope system 10 of the present embodiment, in a case where the treatment tool detected from the endoscopic image disappears from the endoscopic image, after a fixed time has elapsed, the display of the treatment name selection box 73 is displayed on the screen 70A, and thus, the selection of the treatment name is possible. The treatment name selection box 73 is displayed on the screen 70A in a state where one treatment name is selected in advance. Accordingly, it is possible to omit the user's initial selection operation.

In principle, the treatment name selection box 73 displayed on the screen 70A disappears from the screen 70A after a fixed time elapses. Then, the selection of the treatment name is confirmed when the treatment name selection box 73 disappears from the screen 70A. Accordingly, a separate operation of confirming the selection is not required, and thus, it is possible to efficiently input information on the treatment name.

[Report Creation Support]

Creating a report is performed using the user terminal 200. In a case where the report creation support is requested from the user terminal 200 to the endoscope information management system 100, processing of supporting the report creation is started.

First, the examination as the report creation target is selected. The examination as the report creation target is selected on the basis of patient information or the like.

In a case where the examination as the report creation target is selected, the lesion or the like as the report creation target is selected. In this case, the selection screen 130 is provided to the user terminal 200 (refer to FIG. 26). On the selection screen 130, the user designates the card 132A displayed in the detection list display region 132, and selects the lesion or the like as the report creation target.

In a case where the lesion or the like as the report creation target is selected, the detailed input screen 140 is provided to the user terminal 200 (refer to FIG. 27). In this case, the detailed input screen 140 is provided to the user terminal 200 in a state where information is automatically input to the predetermined input fields in advance. Specifically, the detailed input screen 140 is provided in a state where information acquired during the examination is input to the input field for the endoscopic image, the input field for the site, and the input field for the treatment name in advance (refer to FIG. 29). These pieces of information are automatically input on the basis of the information recorded in the database 120. The user corrects the automatically input information as necessary. Further, the user inputs information to other input fields.

In a case where predetermined information is input and the generation of the report is requested, the report is generated in a predetermined format on the basis of the input information. The report generation unit 114C automatically generates the report in a predetermined format, for the lesion or the like selected as the report creation target, on the basis of the information input on the detailed input screen 140. The generated report is provided to the user terminal 200.

### MODIFICATION EXAMPLES

[Modification Example of Display of Site Selection Box]

In the embodiment described above, the site selection box 71 is displayed on the screen 70A with the detection of the specific region as a trigger, but a configuration can be adopted in which the site selection box 71 is displayed on the screen 70A in response to an instruction to start the display from the user. In this case, it is preferable that the site selection box 71 is displayed on the screen 70A in a state where a specific site is selected in advance. Accordingly, it is possible to save the user's time and effort for the site selection, and it is possible to efficiently input the information on the site. For example, as the site to be selected in advance, a site where the examination (observation) is started is set. As described above, in the examination for the large intestine, since the examination is usually started from the ileocecum, the site selection box 71 can be displayed on the screen 70A with the site to which the ileocecum belongs selected in advance.

Further, the instruction method is not particularly limited. For example, the instruction can be given by an operation using a button provided on the operation part 22 of the endoscope 20, an operation using the input device 50 (including foot switch, audio input device, and the like), and the like.

[Modification Example of Site Selection Box]

In the embodiment described above, the site is selected by displaying the schema diagram of the hollow organ as the examination target, but the method of selecting the site in the site selection box 71 is not limited thereto. In addition, for example, options written in text may be displayed in a list, and the user may select the option. For example, in the example of the embodiment described above, a configuration can be adopted in which three of “ascending colon”, “transverse colon”, and “descending colon” are written in text, and are displayed in the site selection box 71 in a list, and the user selects one. Further, for example, a configuration can be adopted in which the text notation and the schema diagram are combined and displayed. Moreover, the site being selected may be separately displayed as text. Accordingly, it is possible to clarify the site being selected.

Further, the method of dividing the sites as the options can be appropriately set according to the type of the hollow organ as the examination target, the purpose of the examination, and the like. For example, in the embodiment described above, the large intestine is divided into three sites, but can be divided into more detailed sites. For example, in addition to “ascending colon”, “transverse colon”, and “descending colon”, “sigmoid colon” and “rectum” can be added as the options. Moreover, each of “ascending colon”, “transverse colon”, and “descending colon” may be classified in more detail, and a more detailed site can be selected.

[Emphasized Display]

It is preferable that the emphasized display of the site selection box 71 is executed at a timing when it is necessary to input the information on the site. For example, as described above, the information on the site is recorded in association with the treatment name. Therefore, it is preferable to select the site according to the input of the treatment name. As described above, the acceptance of the selection of the site is stopped while the selection of the treatment name is being accepted. Therefore, it is preferable that, before the selection of the treatment name is accepted or after the selection of the treatment name is accepted, the site selection box 71 is displayed in an emphasized manner to prompt the selection of the site. Note that, since a plurality of lesion parts are detected in the same site in some cases, it is more preferable to select the site in advance before the treatment. Therefore, for example, it is preferable that the site selection box 71 is displayed in an emphasized manner at a timing when the treatment tool is detected from the image or at a timing when the lesion part is detected from the image, to prompt the selection of the site. The treatment tool and the lesion part are examples of a detection target different from the specific region.

Further, the site selection box 71 may be displayed in an emphasized manner at the timing of switching the site to prompt the selection of the site. In this case, for example, the site switching is detected from the image by using the AI or the trained model. As in the embodiment described above, in the examination for the large intestine, in a case where the site is selected by divided the large intestine into the ascending colon, the transverse colon, and the descending colon, the site switching can be detected by detecting the hepatic flexure (right colon), the splenic flexure (left colon), and the like from the image. For example, switching from the ascending colon to the transverse colon or switching from the transverse colon to the ascending colon can be detected by detecting the hepatic flexure. Further, switching from the transverse colon to the descending colon or switching from the descending colon to the transverse colon can be detected by detecting the splenic flexure.

As described above, as the method of the emphasized display, in addition to the method of displaying the site selection box 71 in an enlarged manner, methods of changing a color from the normal display form, enclosing with a frame, blinking, and the like can be adopted. Further, a method of appropriately combining the methods can be adopted.

Further, instead of or in addition to the method of prompting the selection of the site via the emphasized display, processing of prompting the selection of the site may be performed using an audio guide or the like. Alternatively, the display of prompting the selection of the site on the screen (for example, message, icon, or the like) may be separately performed.

[Other Uses of Information on Site]

In the embodiment described above, a case where the information on the selected site is recorded in association with the information on the treatment name has been described, but the use of the information on the site is not limited thereto. For example, a configuration can be adopted in which the information on the site being selected is recorded in association with the captured endoscopic image. Accordingly, it can be easily discriminated from which site the acquired endoscopic image is captured. Further, classification or the like of the endoscopic image can be performed for each site by using the associated information on the site.

[Selection Operation of Site]

In the embodiment described above, the selection operation of the site is performed by the foot switch, but the selection operation of the site is not limited thereto. In addition, a configuration can be adopted in which the selection operation is performed by an audio input, a gaze input, a button operation, a touch operation on a touch panel, or the like.

[Modification Example of Treatment Name Selection Box]

The treatment names to be displayed as the selectable treatment names in the treatment name selection box 73 may be arbitrarily set by the user. That is, the user may arbitrarily set or edit the table. In this case, it is preferable that the user can arbitrarily set and edit the number, the order, and the default option of treatment names to be displayed. Accordingly, it is possible to build a user-friendly environment for each user.

Further, a selection history may be recorded, and the table may be automatically corrected on the basis of the recorded selection history. For example, on the basis of the history, the display order may be corrected to the descending order of the selection frequency, or the default option may be corrected. Further, for example, the display order may be corrected to an order of newest selection on the basis of the history. In this case, the display is made in the order of the last selected option (previous selected option) displayed at the top, followed by the second-to-last selected option, the third-to-last selected option, and so on. Similarly, on the basis of the history, the last selected option may be corrected to be the default option.

Further, in the options to be displayed in the treatment name selection box 73, items such as “no treatment” and/or “post-selection” can be included in addition to the treatment name. Accordingly, for example, even in a case where the treatment is not performed, information thereof can be recorded. Further, it is possible to cope with a case where an input of the treatment name is performed after the examination, a case where the performed treatment is not included in the options, or the like.

Further, in the embodiment described above, the treatment name selection box 73 is displayed by associating the treatment tools with the treatment name selection boxes in a one-to-one manner, but the treatment name selection box 73 may be displayed by associating one treatment name selection box with a plurality of treatment tools. That is, in a case where a plurality of treatment tools are detected from the image, the treatment name selection box 73 in which the option of the treatment name corresponding to the combination of the plurality of treatment names is displayed is displayed on the screen 70A.

[Display Timing of Treatment Name Selection Box]

In the embodiment described above, after a fixed time has elapsed from the detection of the disappearance of the treatment tool from the image, the treatment name selection box 73 is displayed on the screen 70A, but the timing when the treatment name selection box 73 is displayed is not limited thereto. For example, a configuration can be adopted in which the treatment name selection box 73 is displayed immediately after the disappearance of the treatment tool from the image is detected.

Further, for example, the end of the treatment is detected from the image by using the AI or the trained model, and the treatment name selection box 73 may be displayed on the screen 70A immediately after the detection or after a fixed time has elapsed from the detection.

By displaying the treatment name selection box 73 after treatment rather than during the treatment, it is possible to concentrate on the treatment during the treatment.

[Display of Treatment Name Selection Box]

There are a plurality of types of treatment tools, but it is preferable that, only in a case where a specific treatment tool is detected, the treatment name selection box 73 corresponding to the detected specific treatment tool is displayed on the screen to accept the selection.

For example, depending on the treatment tool, there may be only one executable treatment. For example, regarding a hemostatic pin as one of the treatment tools, there is no executable treatment other than the hemostasis. Therefore, in this case, since there is no room for selection, the display of the treatment name selection box is not necessary.

Note that, for the treatment tool for which there is only one executable treatment, the treatment name may be automatically input when the treatment tool is detected. In this case, instead of displaying the treatment name selection box 73, the treatment name corresponding to the detected treatment tool may be displayed on the screen 70A, and the display of the treatment name disappears after a fixed time has elapsed, thereby confirming the input. Alternatively, a configuration can be adopted in which the treatment name selection box 73 is displayed in combination with the items of “no treatment” and/or “post-selection” to prompt the user to perform the selection.

[Manually Calling Up Treatment Name Selection Box]

The treatment name selection box may be manually called up. Accordingly, the treatment name selection box can be called up at any timing. The instruction method is not particularly limited. For example, a call instruction can be given by an operation using the button provided on the operation part 22 of the endoscope 20, an operation using the input device 50 (including foot switch, audio input device, and the like), and the like. As an example, a configuration can be adopted in which the treatment name selection box is called up by pressing the foot switch for a long time.

Note that, in a case where the treatment name selection box is manually called up, an option determined in advance is displayed. The option to be displayed may be arbitrarily set by the user.

[Detailed Input Screen for Report Creation Support]

In the detailed input screen 140 for the report creation support, it is preferable that the automatically filled input fields are distinguishable from other input fields. For example, the automatically filled input fields are distinguishable from other input fields by being displayed in an emphasized manner. Accordingly, it is possible to clarify that the items are automatically filled, and to call attention to the user.

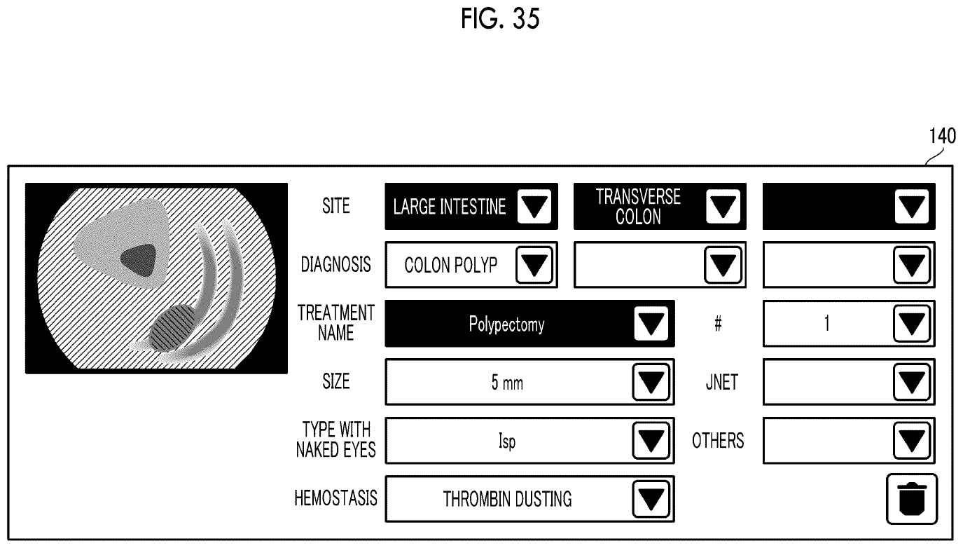

FIG. 35 is a diagram illustrating a modification example of the detailed input screen.

In the example illustrated in the figure, the input field for the site and the input field for the treatment name are displayed in a reversed manner so that the input fields are distinguishable from other input fields. More specifically, a background color and a character color are displayed in a reversed manner so that the input fields are distinguishable from other input fields.

In addition, by making the automatically filled input fields blink, enclosing the automatically filled input fields with a frame, or attaching a caution symbol to the automatically filled input fields, it may be possible to make the automatically filled input fields distinguishable from other input fields.

[Automatic Input]

In the embodiment described above, the information on the site and the information on the treatment name for the lesion or the like as the report creation target are acquired from the database 120, and corresponding input fields are automatically filled, but the method of automatic input is not limited thereto. For example, during the examination, a method can be adopted which records information on the selected site and on the selected treatment name over time (a so-called time log) during the examination, and automatically inputs information on the site, the treatment name, the endoscopic image, and the like by checking with the imaging date and time of the endoscopic image (static image) acquired during the examination. Alternatively, a method can be adopted which records the information on the site and the information on the treatment name in association with the endoscopic image, and automatically inputs the information on the site, the treatment name, the endoscopic image, and the like. In addition, in a case where the endoscopic image is recorded as a video, a method can be adopted which automatically inputs information on the site and on the treatment name from the time information of the video and the information on the time log of the site and the treatment name.

### Second Embodiment

As described above, it is preferable that items to be entered in the report can be input without any time and effort during the examination. The endoscopic image diagnosis support system of the present embodiment is configured such that the information regarding a treatment target (lesion part or the like) can be input during the examination. Specifically, the endoscopic image diagnosis support system is configured such that a specific event related to the treatment is detected, a predetermined selection box is displayed on the screen, and information on a detailed site (position) of the treatment target, information on a size of the treatment target, and the like can be input.

Note that the functions are provided as functions of the endoscopic image processing device. Thus, only the functions in the endoscopic image processing device will be described here.

[Endoscopic Image Processing Device]

As described above, the endoscopic image processing device of the present embodiment is configured such that a specific event is detected, a predetermined selection box is displayed on the screen, and information on a detailed site of the treatment target, information on a size of the treatment target (lesion part or the like), and the like can be input. For example, the specific event is an end of the treatment, a detection of the treatment tool, or the like. In the present embodiment, the detailed site selection box is displayed on the screen in accordance with the detection of the treatment tool. Further, the size selection box is displayed on the screen after the detailed site is selected using the detailed site selection box.

[Detailed Site Selection Box]

The display control unit 64 displays a detailed site selection box 90 on the screen in a case where the treatment tool is detected from the endoscopic image by the treatment tool detection unit 63D.

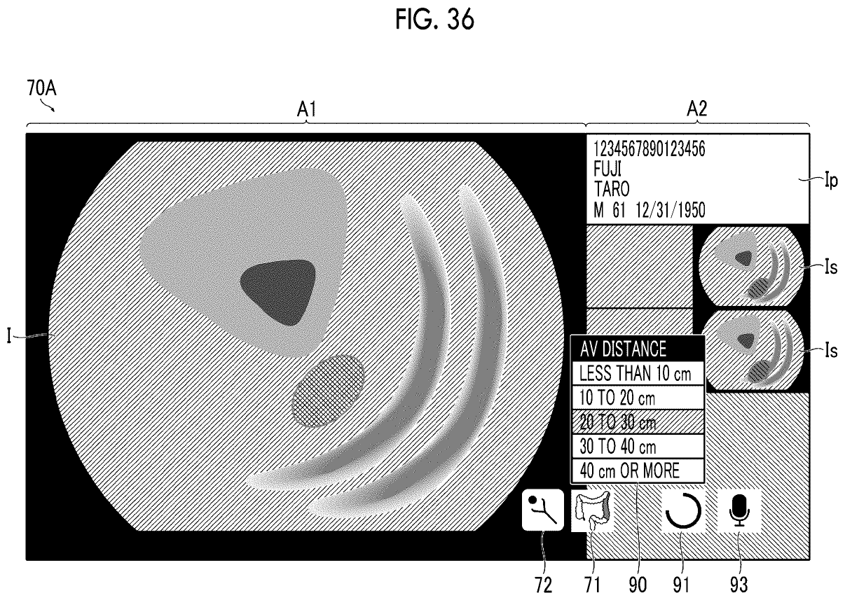

FIG. 36 is a diagram illustrating an example of display of the detailed site selection box.

The detailed site selection box 90 is a region for selecting a detailed site of the treatment target on the screen. The detailed site selection box 90 constitutes an interface for inputting the detailed site of the treatment target on the screen. In the present embodiment, the detailed site selection box 90 is displayed at a predetermined position on the screen 70A in accordance with the detection of the treatment tool. The display position is preferably in the vicinity of the treatment tool detection icon 72. The display control unit 64 displays the detailed site selection box 90 in a pop-up. The region where the detailed site selection box 90 is displayed on the screen is an example of a fifth region.

For example, the detailed site is specified by a distance from an insertion end. Therefore, for example, in a case where the hollow organ of the examination target is the large intestine, the detailed site is specified by a distance from an anal verge. The distance from the anal verge is referred to as an “AV distance”. The AV distance is essentially synonymous with the insertion length.

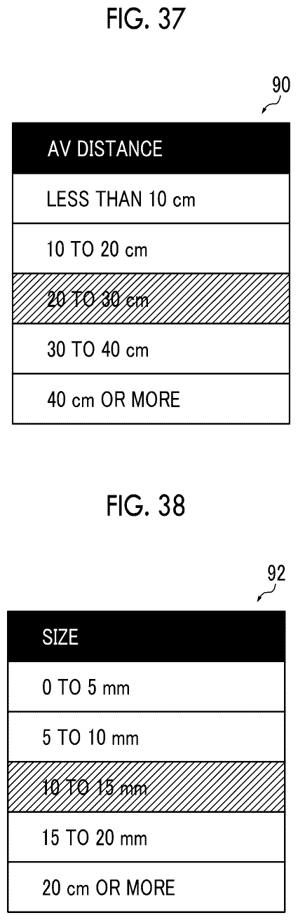

FIG. 37 is a diagram illustrating an example of the detailed site selection box.

As illustrated in the figure, the detailed site selection box 90 is configured by a so-called list box, and selectable AV distances are displayed in a list. In the example illustrated in FIG. 37, a case in which a list of selectable AV distances is displayed in a vertical line is illustrated. A plurality of options regarding the AV distances of the processing target are examples of a plurality of options regarding the treatment target.

For example, the selectable AV distances are displayed in predetermined distance divisions. In the example illustrated in FIG. 37, a case of selecting one option from five distance divisions is illustrated. Specifically, a case of selecting one option from five distance divisions of “less than 10 cm”, “10 to 20 cm (10 cm or more and less than 20 cm)”, “20 to 30 cm (20 cm or more and less than 30 cm)”, “30 to 40 cm (30 cm or more and less than 40 cm)”, and “40 cm or more” is illustrated.

In FIG. 37, an option with a hatched background portion indicates the option being selected. In the example illustrated in FIG. 37, a case where “20 to 30 cm” is selected is illustrated.

In a case where the detailed site selection box 90 is displayed on the screen, the display control unit 64 displays the detailed site selection box 90 on the screen in a state where one option is selected in advance. In the present embodiment, the detailed site selection box is displayed in a state where the option positioned at the top of the list is selected in advance. That is, the option positioned at the top of the list is selected and displayed as the default option. In the example illustrated in FIG. 37, “less than 10 cm” is the default option.

Selection is performed using the input device 50. In the present embodiment, the selection is performed using the foot switch. The selection target is switched in order from the top to the bottom of the list each time the user steps on the foot switch. Moreover, in a case where the foot switch is stepped on after the selection target has reached the bottom of the list, the selection target returns to the top of the list again.

Selection is accepted for a fixed time (T5) from the start of the display of the detailed site selection box 90. In a case where the selection operation (operation of foot switch) is performed within a fixed time from the start of the display, selection is further accepted for a fixed time (T5). That is, the time for which the selection is possible extends. In a case where a state of no operation is continued for a fixed time (T5), the selection is confirmed. That is, the option that is selected at a stage where a fixed time (T5) has elapsed in the state of no operation is confirmed as the option selected by the user. Therefore, for example, in a case where a fixed time (T5) has elapsed in a state of no operation (no selection) after the start of the display of the detailed site selection box 90, the option selected by default is confirmed as the option selected by the user.

As illustrated in FIG. 36, a countdown timer 91 is displayed on the screen 70A such that the remaining time for the selection operation can be known. In FIG. 36, as an example, a case where the countdown timer 91 is displayed as a circle is illustrated. In this case, the color of the circumference is changed over time. The countdown is ended at a stage where the color change has completed one cycle. FIG. 36 illustrates a state where the remaining time is ¼ of time T5. The countdown timer 91 is displayed adjacent to the detailed site selection box 90. The form of the countdown timer 91 is not limited thereto, and may be configured such that, for example, seconds of the remaining time are displayed in numerical values.

The information on the selected (input) detailed site (information on the AV distance) is stored in association with the information on the site being selected, the information on the treatment name to be input (selected) later, and the like. The stored information is used for creating a report. For example, in a case where a report is created by the report creation support unit 114, corresponding input fields are automatically filled.

[Size Selection Box]

In a case where the selection of the detailed site is confirmed, the display control unit 64 displays a size selection box 92 instead of the detailed site selection box 90 on the screen. The region where the size selection box 92 is displayed on the screen is an example of the fifth region. The size selection box 92 is a region for selecting a size of the treatment target (lesion part or the like) on the screen. The size selection box 92 constitutes an interface for inputting the size of the treatment target on the screen.

FIG. 38 is a diagram illustrating an example of the size selection box.

As illustrated in the figure, the size selection box 92 is configured by a so-called list box, and selectable sizes are displayed in a list. In the example illustrated in FIG. 38, a case in which a list of selectable sizes is displayed in a vertical line is illustrated. A plurality of options regarding the size of the processing target are other examples of a plurality of options regarding the treatment target.

For example, the selectable sizes are displayed in predetermined size divisions. In the example illustrated in FIG. 38, a case of selecting one option from five size divisions is illustrated. Specifically, a case of selecting one option from five size divisions of “0 to 5 mm (0 mm or more and less than 5 mm)”, “5 to 10 mm (5 mm or more and less than 10 mm)”, “10 to 15 mm (10 mm or more and less than 15 mm)”, “15 to 20 mm (15 mm or more and less than 20 mm)”, and “20 mm or more” is illustrated.

In FIG. 38, an option with a hatched background portion indicates the option being selected. In the example illustrated in FIG. 38, a case where “10 to 15 mm” is selected is illustrated.

In a case where the size selection box 92 is displayed on the screen, the display control unit 64 displays the size selection box 92 on the screen in a state where one option is selected in advance. In the present embodiment, the detailed site selection box is displayed in a state where the option positioned at the top of the list is selected in advance. That is, the option positioned at the top of the list is selected and displayed as the default option. In the example illustrated in FIG. 38, “0 to 5 mm” is the default option.

Selection is performed using the input device 50. In the present embodiment, the selection is performed using the foot switch. The selection target is switched in order from the top to the bottom of the list each time the user steps on the foot switch. Moreover, in a case where the foot switch is stepped on after the selection target has reached the bottom of the list, the selection target returns to the top of the list again.

Selection is accepted for a fixed time (T6) from the start of the display of the size selection box 92. In a case where the selection operation (operation of foot switch) is performed within a fixed time from the start of the display, selection is further accepted for a fixed time (T6). In a case where a state of no operation is continued for a fixed time (T6), the selection is confirmed.

Similar to the selection of the detailed site, the countdown timer 91 is displayed on the screen 70A such that the remaining time for the selection operation can be known (refer to FIG. 36).

The information on the selected (input) detailed site (information on the AV distance) is stored in association with the information on the site being selected, the information of the detailed site previously input (selected), the information on the treatment name to be input (selected) later, and the like. The stored information is used for creating a report. For example, in a case where a report is created by the report creation support unit 114, corresponding input fields are automatically filled.

In this manner, with the endoscopic image processing device of the present embodiment, the detailed site selection box 90 and the size selection box 92 are displayed on the screen in accordance with a specific event (detection of treatment tool), and the information on the detailed site and the information on the size can be input for the treatment target. Accordingly, it is possible to save time and effort for creating a report.

### Modification Example

[Display Condition]

In the embodiment described above, the detailed site selection box 90 is displayed on the screen with the detection of the treatment tool as a trigger, but the condition of a trigger for the display is not limited thereto. The detailed site selection box 90 may be displayed on the screen with the detection of the end of the treatment as a trigger. Further, the detailed site selection box 90 may be displayed on the screen after a fixed time has elapsed from the detection of the treatment tool or after a fixed time has elapsed from the detection of the end of the treatment.

Further, in the embodiment described above, the size selection box 92 is displayed after the detailed site selection box 90 is displayed, but the order of displaying selection boxes is not particularly limited.

Further, a configuration can be adopted in which the detailed site selection box 90, the size selection box 92, and the treatment name selection box 73 are consecutively displayed in a predetermined order. For example, a configuration can be adopted in which in a case where the treatment end is detected, or in a case where the treatment tool is detected, selection boxes are displayed in order of the detailed site selection box 90, the size selection box 92, and the treatment name selection box 73.

Further, a configuration can be adopted in which each selection box is displayed on the screen with a display instruction via an audio input as a trigger. In this case, a configuration can be adopted in which each selection box is displayed on the screen after waiting for the display instruction via an audio input after the treatment tool is detected. For example, a configuration can be adopted in which in a state where the treatment tool is detected in the image (during recognition of treatment tool), in a case where audio is input, a corresponding selection box is displayed. For example, a configuration can be adopted in which in a case where “AV” is input by audio in a state where the treatment tool is being detected, the detailed site selection box 90 is displayed on the screen, and in a case where “size” is input by audio, the size selection box 92 is displayed on the screen.

In the configuration in which audio can be input, it is preferable that, for example, a predetermined icon is displayed on the screen to indicate to the user that audio can be input. Reference numeral 93 illustrated in FIG. 36 is an example of an icon. In a case where this icon (audio input icon) 93 is displayed on the screen, audio can be input. Therefore, for example, in the example described above, in a case where the treatment tool is detected, the audio input icon 93 is displayed on the screen.

Note that the audio input technique including audio recognition is a well-known technique, so detailed description thereof will be omitted.

[Default Option]

In the embodiment described above, the option positioned at the top of the list is used as the default option, but a configuration can be adopted in which the default option is dynamically changed on the basis of various kinds of information. For example, for the detailed site, the default option can be changed according to the site being selected. Further, for example, a configuration can be adopted in which in a case where an insertion length is separately measured, information on the measured insertion length is acquired, and the default option is set on the basis of the acquired information on the insertion length. In this case, a measurement unit for the insertion length is separately provided. Further, for the size, for example, a configuration can be adopted in which a size is measured by image measurement, information on the measured size is acquired, and the default option is set on the basis of the acquired information on the size. In this case, a function of an image measurement unit is separately provided.

[Selection Method]

In the embodiment described above, the option is selected using the foot switch, but the method of selecting the option is not limited thereto. For example, a configuration can be adopted in which the option is selected by an audio input device instead of the foot switch, or in combination with the foot switch.

In a case of the selection via the audio input, for example, the selection can be confirmed at the same time as the selection. That is, a configuration can be adopted in which the selection is confirmed without any waiting time. In this case, selection of an option via an audio input is confirmed at the same time as completion of the audio input.

Note that a configuration can be adopted in which in a case where selection via an audio input is adopted, the display of the selection box is performed by the audio input. In this case, a configuration can be adopted in which selection of the option is performed at the same time as a display instruction for each selection box. For example, a configuration can be adopted in which in a case where “AV 30 cm” is input by audio in a state where the treatment tool is being detected, the detailed site selection box 90 is displayed on the screen, and “30 to 40 cm” is selected as the option. Accordingly, the user can check the input information on the screen. In a case of correction, audio for the option to be corrected is input while the detailed site selection box 90 is displayed. Further, in a case of combination with the foot switch, the option can be switched by the foot switch.

### Third Embodiment

In the second embodiment described above, a configuration is adopted in which an event regarding the treatment is detected, a predetermined selection box is displayed on the screen, and predetermined information regarding the treatment target can be input. It is preferable that, regardless of the presence or absence of the treatment, items to be entered in the report can be input without any time and effort during the examination. The endoscopic image diagnosis support system of the present embodiment is configured such that the information regarding a region of interest such as a lesion part can be appropriately input during the examination.

Note that the functions are provided as functions of the endoscopic image processing device. Thus, only the functions in the endoscopic image processing device will be described here.

[Endoscopic Image Processing Device]

The endoscopic image processing device of the present embodiment is configured such that, during the examination, a predetermined selection box is displayed on the screen with the detection of a specific event as a trigger, and information regarding a region of interest such as a lesion part can be selectively input. Specifically, the detailed site selection box or the size selection box is displayed on the screen according to the acquisition of a key image. Here, the key image is an image that can be used for diagnosis after examination, or an image that can be used (attached) for a report to be created after examination. That is, the key image is an image (candidate image) as a candidate for the image to be used in diagnosis, a report, or the like. Therefore, the endoscope information management device 110 acquires a static image regarded as a key image, as a static image to be used in a report. Therefore, the static image acquired as the key image is automatically input to the input field 140A (in a case of one key image). The static image acquired as the key image is recorded with predetermined identification information (information indicating that the static image is a key image) added thereto in order to distinguish the static image from other static images.

[Display of Detailed Site Selection Box and Size Selection Box]

As described above, in the endoscopic image processing device of the present embodiment, the detailed site selection box or the size selection box is displayed on the screen according to the acquisition of a key image.

In the present embodiment, in a case where “key image” is input by audio immediately after the static image is captured, the static image obtained by imaging is designated as the key image, and the key image is acquired.

In a case where the key image is acquired, the display control unit 64 displays the detailed site selection box 90 on the screen (refer to FIG. 36). The detailed site selection box 90 is displayed on the screen in a state where one option is selected in advance. The user performs a selection operation via the foot switch or the audio input. In a case where a state of no operation (no selection) is continued for a fixed time (T5), the selection is confirmed. Note that, in the present embodiment, a plurality of options for the AV distance displayed in the detailed site selection box 90 are examples of a plurality of options regarding a region of interest.

In a case where the selection of the detailed site is confirmed, the display control unit 64 displays a size selection box 92 instead of the detailed site selection box 90 on the screen. The size selection box 92 is displayed on the screen in a state where one option is selected in advance. The user performs a selection operation via the foot switch or the audio input. In a case where a state of no operation (no selection) is continued for a fixed time (T6), the selection is confirmed. Note that, in the present embodiment, a plurality of options for the size displayed in the size selection box 92 are examples of a plurality of options regarding a region of interest.

In this manner, with the endoscopic image processing device of the present embodiment, the detailed site selection box 90 and the size selection box 92 are displayed on the screen in accordance with the acquisition of the key image, and regardless of the presence or absence of the treatment, the information on the detailed site and the information on the size can be input for the region of interest such as a lesion part. Accordingly, it is possible to save time and effort for creating a report.

The information input (selected) using each selection box is stored in association with the information on the site being selected and the information on the key image. The stored information is used for creating a report. For example, in a case where a report is created by the report creation support unit 114, corresponding input fields are automatically filled.

Note that the modification example illustrated in the second embodiment is also applied to the present embodiment.

### Modification Example

[Method of Acquiring Key Image]

In the embodiment described above, the key image is acquired in a case where “key image” is input by audio immediately after the static image is captured, but the method of acquiring the key image is not limited thereto.

For example, a configuration can be adopted in which a key image is acquired in a case where a static image is captured by performing a predetermined operation. For example, a configuration can be adopted in which a key image is acquired in a case where a static image is captured by pressing a specific button provided on the operation part 22 of the endoscope 20. Alternatively, a configuration can be adopted in which a key image is acquired in a case where a static image is captured by inputting a predetermined keyword using audio. For example, a configuration can be adopted in which a key image is acquired in a case where a static image is captured by inputting “key image” using audio before imaging.

Further, for example, a configuration can be adopted in which a key image is acquired by performing a predetermined operation after a static image is captured. For example, a configuration can be adopted in which a static image obtained by imaging is acquired as a key image by pressing a specific button provided on the operation part 22 of the endoscope 20 immediately after the static image is captured. Alternatively, a configuration can be adopted in which a static image obtained by imaging is acquired as a key image by an operation of stepping on the foot switch for a fixed time (pressing for a long time) immediately after the static image is captured. Alternatively, a configuration can be adopted in which a key image is acquired in a case where a predetermined keyword is input by audio after a static image is captured. For example, a configuration can be adopted in which in a case where “key image” is input by audio immediately after a static image is captured, the static image obtained by imaging is acquired as the key image.

Further, a configuration may be adopted in which, after a static image is captured, whether or not to adopt the captured image as a key image can be selected. For example, a configuration can be adopted in which in a case where a predetermined operation is performed after a static image is captured, a menu for selecting the use of the image is displayed on the screen, and a key image can be selected as one of options in the menu. As the predetermined operation, for example, an operation of stepping on the foot switch for a fixed time or longer is exemplified. In this case, in a case where the foot switch is stepped on for a fixed time or longer immediately after a static image is captured, a menu for the use of the image is displayed, and an option is displayed by the foot switch or the audio input. For example, a configuration can be adopted in which the menu is displayed each time the static image is captured. In this case, acceptance of selection is performed for a fixed time, and in a case where the selection operation is not performed, the display of the menu disappears.

The acquired key image is recorded in association with the information on the site being selected. Further, the key image acquired at the time of the treatment (key image acquired during the treatment, within a certain period before the treatment, or within a certain period after the treatment) is recorded in association with the input treatment name. In this case, the key image is also recorded in association with the information on the site being selected.

Further, a configuration can be adopted in which the key image is automatically acquired with a predetermined event as a trigger. For example, a configuration can be adopted in which a key image is automatically acquired in accordance with an input of a site and/or an input of a treatment name. Specifically, the key image is acquired as follows.

(1) Case of Acquiring Key Image According to Input of Site

In this case, the most recently captured static image is acquired as a key image according to an input of a site. That is, the most recent static image in terms of time is selected as a key image from among the static images captured before a time point when the site is input.

As another form, the oldest static image in terms of time can be selected as a key image from among the static images captured after a time point when the site is input. That is, the first captured static image after the site is input is selected as a key image.

As still another form, a configuration can be adopted in which an image (one frame of video) captured at a time point when an input of a site is performed is automatically acquired as a key image. In this case, a configuration can be adopted in which a plurality of frames before and after the time point when the input of the site is performed is acquired as a plurality of key images. Further, a configuration can be adopted in which an image with the best image quality from among the images is automatically extracted, and is automatically acquired as a key image. The image with a good image quality is an image with no blurriness and blurs and with proper exposure. Therefore, for example, an image with exposure in a proper range and with high sharpness (image with no blurriness and blurs) is automatically extracted as an image with good image quality.

The key image acquired according to the input of the site is recorded in association with the information on the site being selected.

(2) Case of Acquiring Key Image According to Input of Treatment Name

Also in this case, the most recently captured static image is acquired as a key image according to an input of a treatment name. That is, the most recent static image in terms of time is selected as a key image from among the static images captured before a time point when the treatment name is input.

As another form, the oldest static image in terms of time can be selected as a key image from among the static images captured after a time point when the treatment name is input. That is, the first captured static image after the treatment name is input is selected as a key image.

As still another form, a configuration can be adopted in which an image captured at a time point when an input of a treatment name is performed is automatically acquired as a key image. In this case, a configuration can be adopted in which a plurality of frames before and after the time point when the input of the treatment name is performed are acquired as a plurality of key images. Further, a configuration can be adopted in which an image with the best image quality from among the images is automatically extracted, and is automatically acquired as a key image.

The key image acquired according to the input of the treatment name is recorded in association with the information on the treatment name. In this case, the key image is also recorded in association with the information on the site being selected.

[Use of Key Image]

As described above, the report creation support unit 114 of the endoscope information management device 110 automatically inputs the key image to the input field 140A. A plurality of key images may be acquired. That is, a plurality of key images may be acquired as candidates for use in the report. In this case, for example, the report creation support unit 114 displays a plurality of acquired key images in a list on the screen, and accepts selection of the key image to be used in the report. Then, the selected key image is automatically input to the input field 140A.

Further, a video may be attached to the report. In this case, for example, a static image (one frame) constituting one scene of the video can be used as a key image. As the scene (one frame) to be used as the key image, for example, the first scene (first frame) of the video can be used.

Further, in a case where a video is attached to the report, for example, a configuration can be adopted in which in a case where “key image” is input by audio immediately after the video is captured, a key image is automatically acquired from the video. In addition, for example, as described in the modification example, a configuration can be adopted in which in a case where a predetermined operation is performed before the start of the imaging or after the end of the imaging, a key image is automatically acquired from the video.

### Fourth Embodiment

In the endoscopic image diagnosis support system of the present embodiment, insertion of an endoscope into a body cavity and pulling-out of an endoscope from the body cavity are detected from an image, and notification thereof is given. Further, the detected information is included in the examination information, and managed.

The functions are provided as functions of the endoscopic image processing device. Thus, only the functions in the endoscopic image processing device will be described here.

[Detection of Insertion and Pulling-Out of Endoscope]

As described above, insertion and pulling-out of the endoscope are detected from the image. This processing is performed by the image recognition processing unit 63.

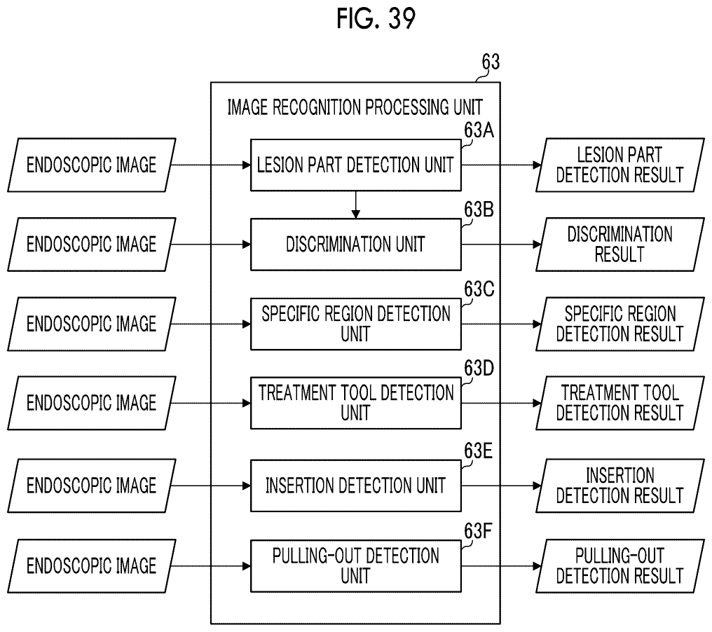

FIG. 39 is a block diagram of main functions of the image recognition processing unit.

As illustrated in the figure, the image recognition processing unit 63 of the present embodiment further includes functions of an insertion detection unit 63E and a pulling-out detection unit 63F.

The insertion detection unit 63E detects insertion of the endoscope into the body cavity, from the endoscopic image. In the present embodiment, insertion into the large intestine via the anus is detected.

The pulling-out detection unit 63F detects pulling-out of the endoscope from the body cavity, from the endoscopic image. In the present embodiment, pulling-out from the body cavity via the anus is detected.

The insertion detection unit 63E and the pulling-out detection unit 63F are configured by the AI or the trained model trained using a machine learning algorithm or deep learning.

Specifically, the insertion detection unit 63E is configured by the AI or the trained model trained to detect the insertion of the endoscope into the body cavity, from the endoscopic image. The pulling-out detection unit 63F is configured by the AI or the trained model trained to detect the pulling-out of the endoscope from the body cavity, from the endoscopic image.

[Notification and Management of Detection]

In a case where the insertion of the endoscope is detected by the insertion detection unit 63E, a predetermined icon is displayed on the screen of the display device 70, and notification of the detection of the insertion is given. Similarly, in a case where the pulling-out of the endoscope is detected by the pulling-out detection unit 63F, a predetermined icon is displayed on the screen of the display device, and notification of the detection of the pulling-out is given.

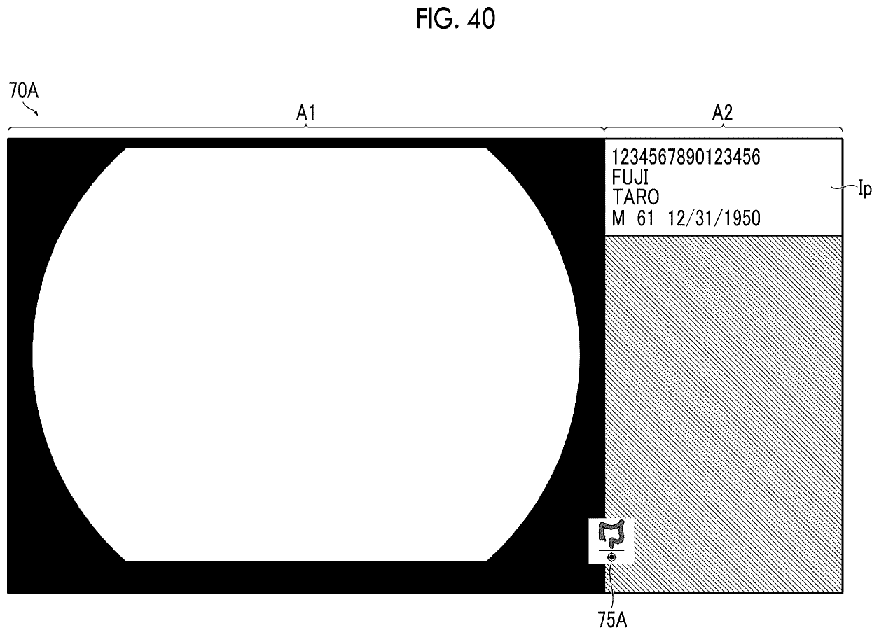

FIG. 40 is a diagram illustrating an example of display of a screen before the endoscope is inserted.

As illustrated in the figure, before the insertion of the endoscope, an icon (hereinafter, referred to as “outside-body icon”) 75A indicating that the endoscope is outside of the body (before insertion) is displayed on the screen 70A. The outside-body icon 75A is displayed at the same position as the position where the site selection box is displayed.

The user can check that the endoscope is not inserted by visually recognizing the outside-body icon 75A.

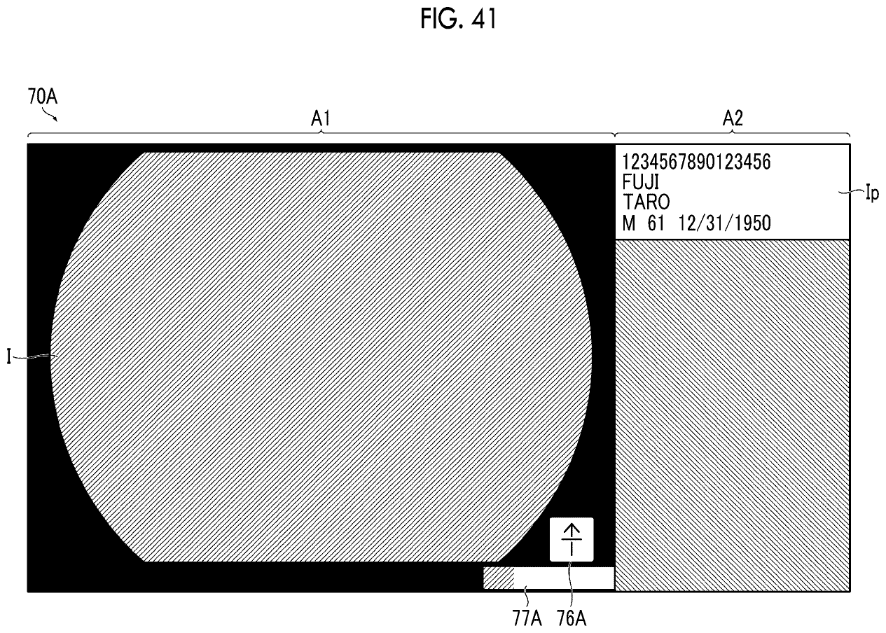

FIG. 41 is a diagram illustrating an example of display of a screen in a case where the insertion of the endoscope is detected.

As illustrated in the figure, in a case where insertion of the endoscope is detected, an icon (hereinafter, referred to as “insertion detection icon”) 76A indicating that the endoscope is inserted is displayed on the screen 70A. The insertion detection icon 76A is displayed at the same position as the position where the treatment tool detection icon 72 is displayed.

Further, a progress bar 77A is displayed on the screen at the same time as the insertion detection icon 76A is displayed. The progress bar 77A indicates the remaining time until the insertion is confirmed. In a case of canceling the detection of the insertion, the user performs a predetermined cancel operation before the progress bar 77A extends to the end. For example, an operation of pressing the foot switch for a long time is performed. Note that “pressing for a long time” is an operation of continuously pressing the foot switch for a fixed time or longer (for example, two seconds or longer).

In this manner, in the endoscopic image diagnosis support system of the present embodiment, an automatically detected result can be canceled. The cancellation is accepted only for a certain period, and is automatically confirmed after the period has elapsed. Accordingly, the user's time and effort for confirming the detection of the insertion can be saved.

The progress bar 77A is displayed at the same position as that of the progress bar 74 displayed in a case of selecting the treatment name.

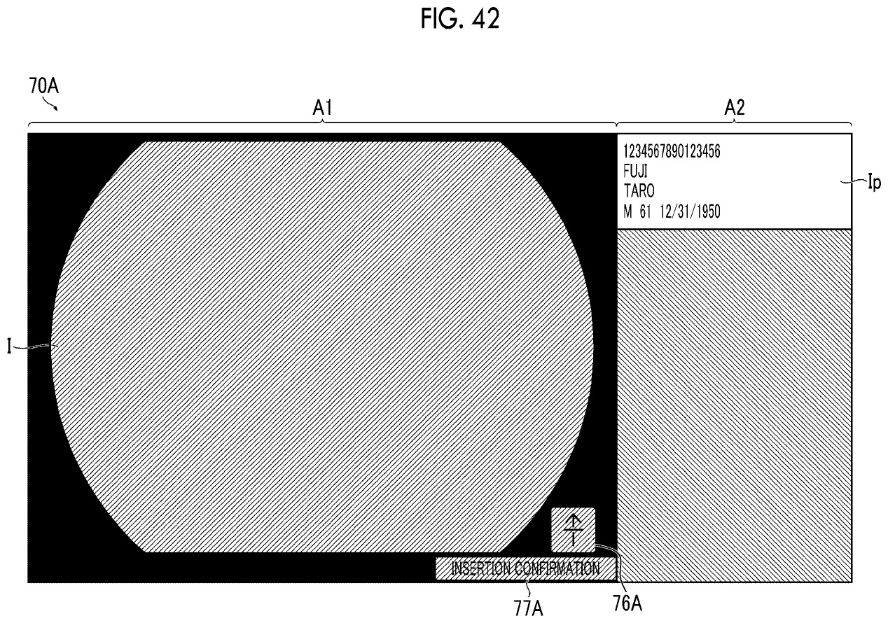

FIG. 42 is a diagram illustrating an example of display of a screen in a case where the detection of the insertion of the endoscope is confirmed.

As illustrated in the figure, text “insertion confirmation” is displayed at the display position of the progress bar 77A, and indicates that the insertion is confirmed. Further, the color (background color) of the insertion detection icon 76A is also changed, and indicates that the insertion is confirmed.

Even after the insertion is confirmed, the insertion detection icon 76A and the progress bar 77A are continuously displayed on the screen for a fixed time. Then, after a fixed time has elapsed from the confirmation, the display disappears from the screen.

In a case where the detection of the insertion is confirmed, information indicating that the detection of the insertion is confirmed is output to the endoscope information management system 100.

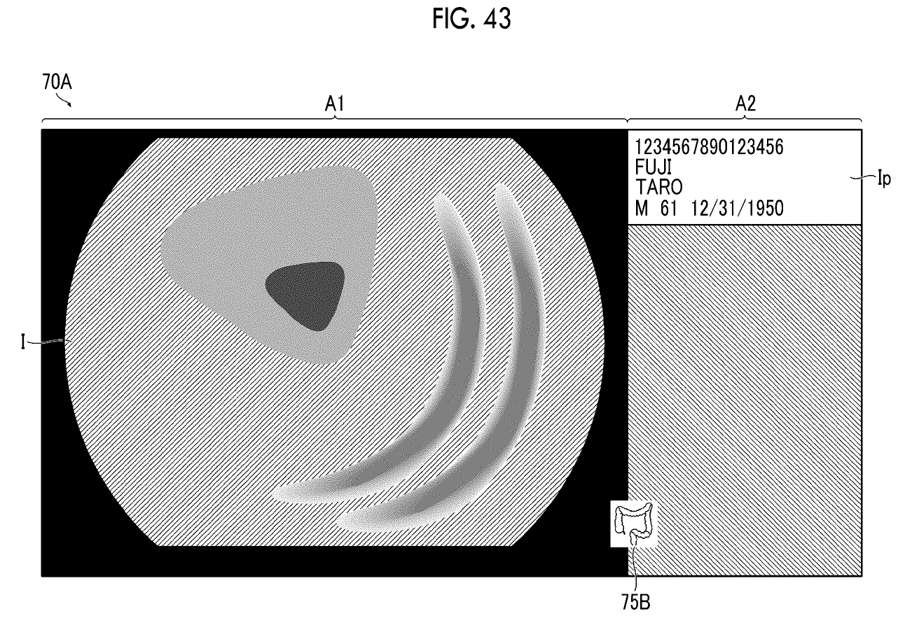

FIG. 43 is a diagram illustrating an example of display of a screen after the detection of the insertion of the endoscope is confirmed.

As illustrated in the figure, in a case where the insertion of the endoscope is confirmed, an icon (hereinafter, referred to as “inside-body icon”) 75B indicating that the endoscope is inserted into the body is displayed on the screen 70A. The inside-body icon 75B has, for example, the same design as the display of the site selection box in a state where no site is selected. The inside-body icon 75B is displayed at the same position as the position where the outside-body icon 75A is displayed (position where the site selection box is displayed).

The user can check that the endoscope is in a state of being inserted into the body by visually recognizing the inside-body icon 75B.

After that, the site selection box 71 is displayed on the screen due to the detection of the ileocecum (refer to FIG. 13).

Note that a configuration can be adopted in which the site selection box 71 is displayed manually. In a case where the display is manually performed, for example, a configuration can be adopted in which the site selection box 71 is displayed by the next operation. That is, a configuration can be adopted in which in a case where the user manually inputs that the endoscope has reached the ileocecum, the site selection box 71 is displayed. Note that a manual input of various kinds of information by the user is referred to as a user input.

The manual input of the ileocecum being reached is performed by, for example, an operation via a button provided on the operation part 22 of the endoscope 20, an operation via the input device 50 (including the foot switch), and the like.

In a case where the user manually inputs that the endoscope has reached the ileocecum, it is preferable to notify of the manual input. Further, it is preferable to adopt a configuration in which the input can be canceled.

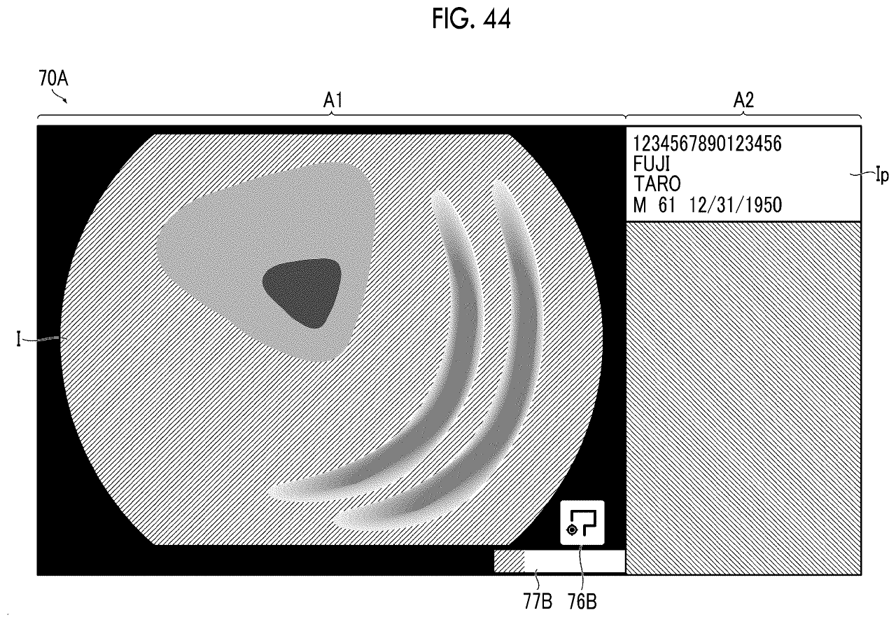

FIG. 44 is a diagram illustrating an example of display of a screen in a case where the ileocecum being reached is manually input.

As illustrated in the figure, in a case where the user manually inputs that the endoscope has reached the ileocecum, an icon (hereinafter, referred to as “ileocecum reached icon”) 76B indicating that the ileocecum being reached is manually input is displayed. The ileocecum reached icon 76B is displayed at the same position as the position where the treatment tool detection icon 72 is displayed.

Further, a progress bar 77B is displayed on the screen at the same time as the ileocecum reached icon 76B is displayed. The progress bar 77B indicates the remaining time until the ileocecum being reached is confirmed. In a case of canceling the manual input of the ileocecum being reached, the user performs a predetermined cancel operation before the progress bar 77B extends to the end. For example, an operation of pressing the foot switch for a long time is performed.

The progress bar 77B is displayed at the same position as that of the progress bar 74 displayed in a case of selecting the treatment name.

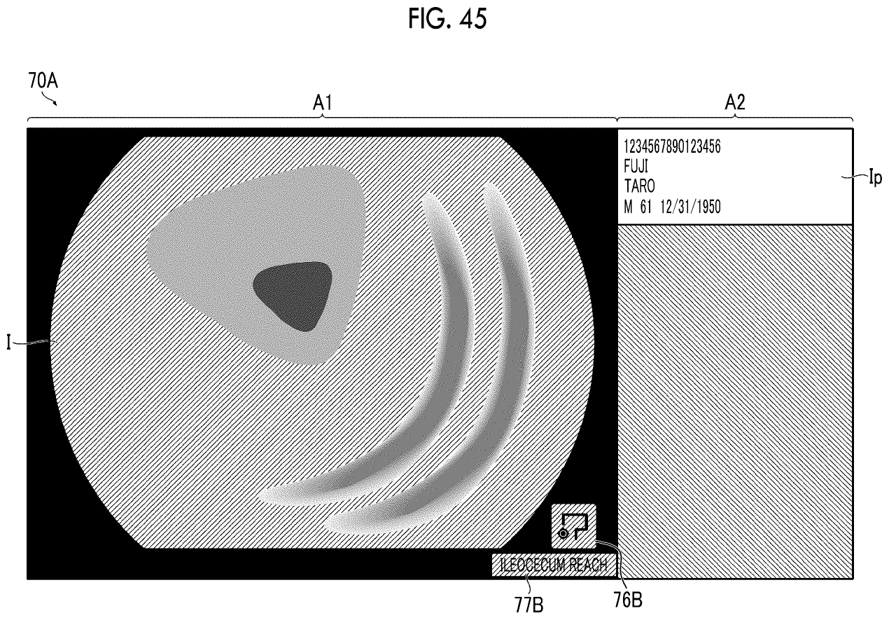

FIG. 45 is a diagram illustrating an example of display of a screen in a case where the ileocecum being reached is confirmed.

As illustrated in the figure, text “ileocecum being reached” is displayed at the display position of the progress bar 77B, and indicates that the ileocecum being reached is confirmed. Further, the color (background color) of the ileocecum reached icon 76B is also changed, and indicates that the ileocecum being reached is confirmed.

Even after the confirmation, the ileocecum reached icon 76B and the progress bar 77B are continuously displayed on the screen for a fixed time, and then disappear from the screen.

In a case where the ileocecum reached icon 76B and the progress bar 77B disappear from the screen, the site selection box 71 is displayed on the screen (refer to FIG. 13).

In this manner, in the present embodiment, the ileocecum being reached can be manually input. Then, the site selection box 71 is displayed on the screen according to the manual input of the ileocecum being reached. Therefore, in the present embodiment, the operation of manually inputting the ileocecum being reached corresponds to an operation of instructing to display the site selection box 71.

As in the present embodiment, in addition to the automatic detection of the ileocecum, by manually inputting the ileocecum being reached, it is possible to cope with a case where the ileocecum cannot be automatically detected. Moreover, by adopting a configuration in which the input can be canceled, an erroneous input can also be suppressed.

Note that it is preferable that the manual input of the ileocecum being reached is accepted after the insertion of the endoscope is confirmed. That is, it is preferable that the ileocecum being reached cannot be manually input until the insertion of the endoscope is confirmed. Accordingly, an erroneous input can be suppressed. Also in a case where the ileocecum is automatically detected, it is preferable to start the detection after the insertion of the endoscope is confirmed. Accordingly, erroneous detection can be suppressed.

In a case where the ileocecum being reached is confirmed, information indicating that the ileocecum being reached is confirmed is output to the endoscope information management system 100.

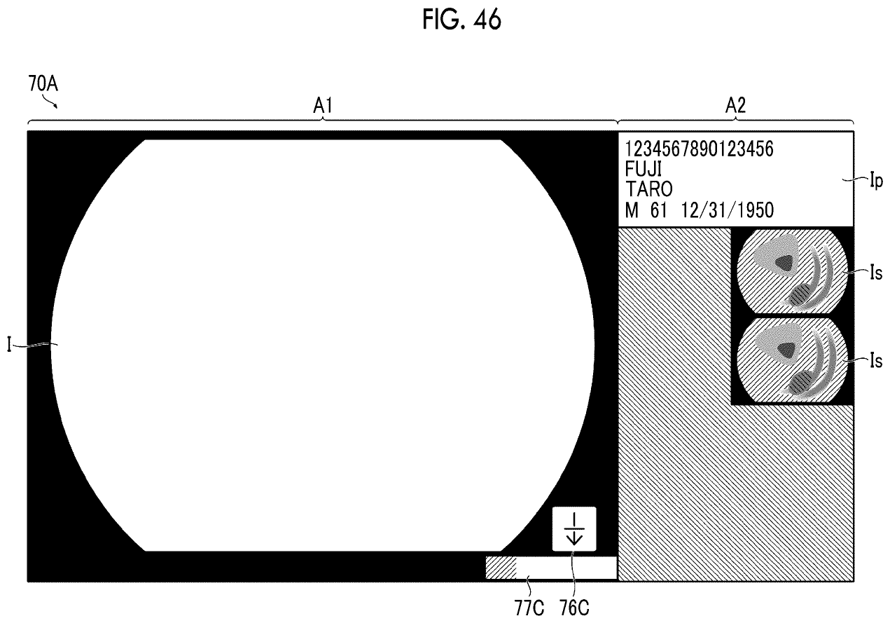

FIG. 46 is a diagram illustrating an example of display of a screen in a case where the pulling-out of the endoscope is detected.

As illustrated in the figure, in a case where the pulling-out of the endoscope from the body is detected, an icon (hereinafter, referred to as “pulling-out detection icon”) 76C indicating that the endoscope is pulled out is displayed on the screen 70A. The pulling-out detection icon 76C is displayed at the same position as the position where the insertion detection icon 76A is displayed (position where the treatment tool detection icon 72 is displayed).

Further, a progress bar 77C is displayed on the screen at the same time as the pulling-out detection icon 76C is displayed. The progress bar 77C indicates the remaining time until the pulling-out is confirmed. In a case of canceling the detection of the pulling-out, the user performs a predetermined cancel operation before the progress bar 77C extends to the end. For example, an operation of pressing the foot switch for a long time is performed.

In this manner, in the endoscopic image diagnosis support system of the present embodiment, an automatically detected result can be canceled. The cancellation is accepted only for a certain period, and is automatically confirmed after the period has elapsed. Accordingly, the user's time and effort for confirming the detection of the pulling-out can be saved.

The progress bar 77C is displayed at the same position as that of the progress bar 74 displayed in a case of selecting the treatment name.

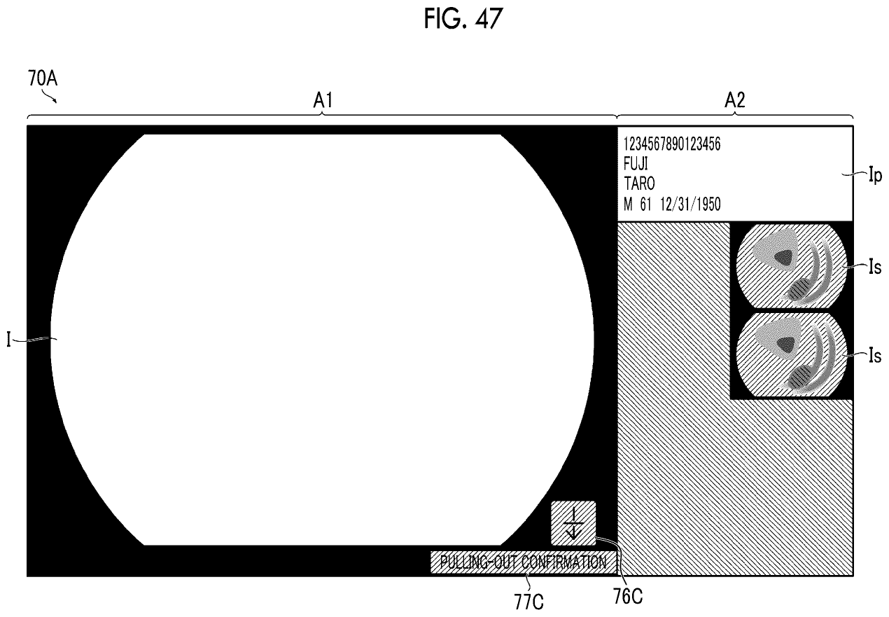

FIG. 47 is a diagram illustrating an example of display of a screen in a case where the detection of the pulling-out of the endoscope is confirmed.

As illustrated in the figure, text “pulling-out confirmation” is displayed at the display position of the progress bar 77C, and indicates that the pulling-out is confirmed. Further, the color (background color) of the pulling-out detection icon 76C is also changed, and indicates that the pulling-out is confirmed.

Even after the pulling-out is confirmed, the pulling-out detection icon 76C and the progress bar 77C are continuously displayed on the screen for a fixed time. Then, after a fixed time has elapsed from the confirmation, the display disappears from the screen.

In a case where the pulling-out detection icon 76C and the progress bar 77C disappear from the screen, the outside-body icon 75A is displayed on the screen (refer to FIG. 40). The user can check that the endoscope is in a state of being pulled out of the body (state of non-insertion) by visually recognizing the outside-body icon 75A.

In a case where the detection of the pulling-out is confirmed, information indicating that the detection of the pulling-out is confirmed is output to the endoscope information management system 100.

In this manner, with the endoscopic image diagnosis support system of the present embodiment, insertion of the endoscope into the body cavity and the pulling-out of the endoscope from the body cavity are automatically detected from the image, and notification thereof is given on the screen.

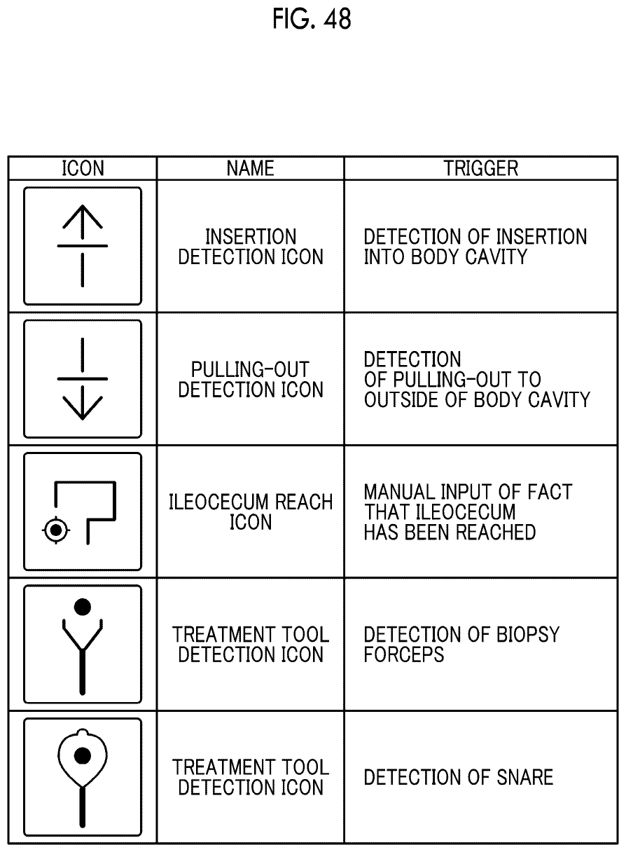

FIG. 48 is a diagram illustrating a list of icons displayed on the screen.

Each icon is displayed at the same position on the screen. That is, each icon is displayed in the vicinity of the position where the treatment tool 80 appears in the endoscopic image I displayed in the main display region A1. Note that, in each figure, another example of the treatment tool detection icon is illustrated.

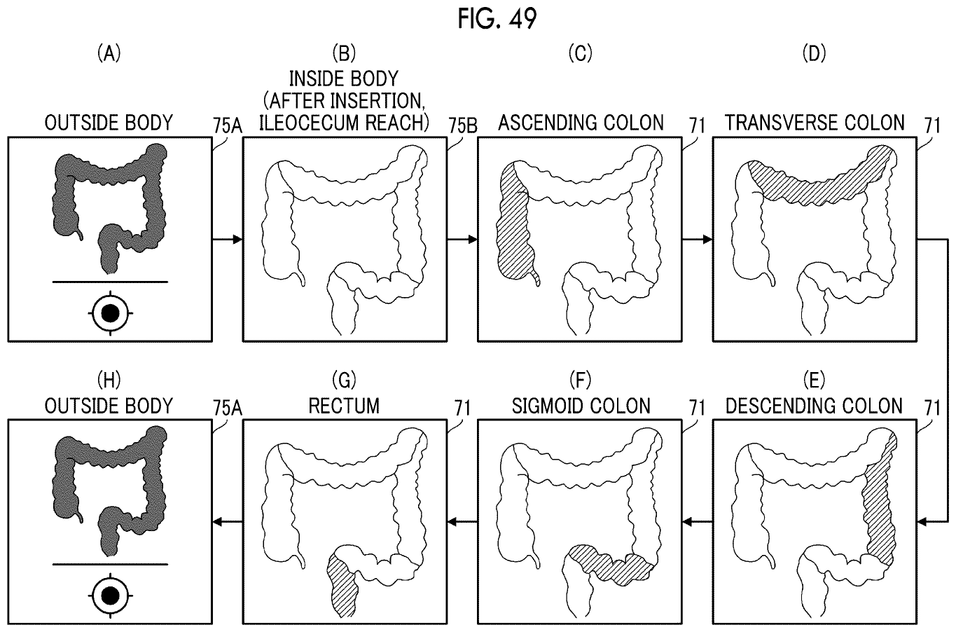

FIG. 49 is a diagram illustrating an example of switching information displayed at the display position of the site selection box.

Each figure illustrates an example of a case where five sites (ascending colon, transverse colon, descending colon, sigmoid colon, and rectum) can be selected in the site selection box 71.

(A) of FIG. 49 illustrates information displayed at the display position of the site selection box in a case where the endoscope is outside of the body cavity (case of non-insertion). As illustrated in the figure, in a case where the endoscope is outside of the body cavity, the outside-body icon 75A is displayed at the display position of the site selection box.

(B) of FIG. 49 illustrates information displayed at the display position of the site selection box in a case where the endoscope is inserted into the body cavity. As illustrated in the figure, in a case where it is detected that the endoscope is inserted into the body cavity, instead of the outside-body icon 75A, the inside-body icon 75B is displayed at the display position of the site selection box.

(C) of FIG. 49 illustrates information displayed at the display position of the site selection box in a case where the ileocecum is detected and in a case where the ileocecum being reached is manually input. As illustrated in the figure, in a case where the ileocecum is detected from the image, or in a case where the ileocecum being reached is manually input, instead of the inside-body icon 75B, the site selection box 71 is displayed. In this case, in the schema diagram, the site selection box 71 is displayed in a state where the ascending colon is selected.

(D) of FIG. 49 illustrates the display of the site selection box 71 in a case where the transverse colon is selected. As illustrated in the figure, in the schema diagram, the display is switched to the state where the transverse colon is selected.

(E) of FIG. 49 illustrates the display of the site selection box 71 in a case where the descending colon is selected. As illustrated in the figure, in the schema diagram, the display is switched to the state where the descending colon is selected.

(F) of FIG. 49 illustrates the display of the site selection box 71 in a case where the sigmoid colon is selected. As illustrated in the figure, in the schema diagram, the display is switched to the state where the sigmoid colon is selected.

(G) of FIG. 49 illustrates the display of the site selection box 71 in a case where the rectum is selected. As illustrated in the figure, in the schema diagram, the display is switched to the state where the rectum is selected.

(I) of FIG. 49 illustrates information displayed at the display position of the site selection box in a case where the endoscope is pulled out of the body cavity. As illustrated in the figure, in a case where it is detected that the endoscope is pulled out of the body cavity, the outside-body icon 75A is displayed at the display position of the site selection box.

### Modification Example

[Manual Input of Insertion and/or Pulling-Out]

In the embodiment described above, the insertion of the endoscope into the body cavity and the pulling-out of the endoscope from the body cavity are automatically detected from the image, but a configuration can be adopted in which the insertion of the endoscope into the body cavity and/or the pulling-out of the endoscope from the body cavity can be manually input. For example, a configuration can be adopted in which the insertion and/or the pulling-out is manually input by an operation using the button provided on the operation part 22 of the endoscope 20, an operation using the input device 50 (including foot switch, audio input device, and the like), and the like. Accordingly, it is possible to manually cope with a case where the detection cannot be automatically performed or the like.

[Recording of Image]

The image (video and static image) acquired during the examination can be held in association with the examination information. In this case, for example, the image can be held by being divided into sections such as “from the insertion confirmation to the ileocecum being reached” and “from the ileocecum being reached to the pulling-out confirmation”.

Further, the image acquired during a period from the ileocecum being reached to the pulling-out confirmation can be held in association with the information on the site. Accordingly, this makes it easier to specify an image in a case of generating a report.

[Ileocecum Reached Icon]

Even in a case where the ileocecum is automatically detected, the ileocecum reached icon 76B may be displayed on the screen. In this case, in a case where the ileocecum is detected, the ileocecum reached icon 76B is displayed on the screen for a fixed time.

Further, in a case where the ileocecum reached icon 76B is displayed even in a case where the ileocecum is automatically detected, it is preferable that the detection can be canceled. Accordingly, it is possible to prevent the site selection box from being displayed due to the erroneous detection. The cancellation is accepted for a fixed time from the start of the display of the ileocecum reached icon 76B, and in a case where there is no cancellation, the detection of the ileocecum is confirmed. Further, in a case where the detection is confirmed, the site selection box is displayed. Further, similar to the case of the manual input, it is preferable to display the progress bar on the screen during the period for accepting the cancellation.

### Fifth Embodiment

As described above, by holding the information on the site during the examination (during the observation), various kinds of information obtained during the examination can be recorded in association with the information on the site. For example, in a case where the recognition processing is performed on the endoscopic image, a result of the recognition processing can be recorded in association with the information on the site.

Further, by recording various kinds of information obtained during the examination in association with the information on the site, these pieces of information can be extracted or presented on a site-by-site basis after the examination. For example, by recording the result of the recognition processing performed during the examination in association with the information on the site, the result of the recognition process can be extracted or presented on a site-by-site basis.

Hereinafter, the endoscopic image diagnosis support system having a function of recording the result of the recognition processing performed during the examination in association with the information on the site and a function of outputting the series of recognition processing results in a predetermined format will be described. Note that the functions are provided as functions of the endoscopic image processing device. Therefore, in the following, only the above-described functions of the endoscopic image processing device will be described.

[Endoscopic Image Processing Device]

[Configuration]

In the present embodiment, as the recognition processing, a case of performing processing of determining the severity of inflammatory bowel disease (IBD), particularly ulcerative colitis (UC), from the endoscopic image will be described as an example. In particular, a case of determining the severity of the ulcerative colitis using Mayo Endoscopic Subscore (hereinafter, referred to as “Mayo score” or “MES”) will be described as an example.

The Mayo score (MES) is one of indices representing the severity of the ulcerative colitis, and indicates classifications of endoscopic findings for the ulcerative colitis. The Mayo score is classified into the following four grades.
Grade 0: normal or inactive (remission) findings
Grade 1: mild disease (erythema, decreased vascular pattern, mild hemorrhage (friability))
Grade 2: moderate disease (marked erythema, absent vascular pattern, hemorrhage (friability), erosions)
Grade 3: severe disease (spontaneous bleeding, ulceration)

In the present embodiment, the recognition processing is performed on the static image captured during the examination (during the observation), and the Mayo score is determined. The result (determination result of the Mayo score) of the recognition processing is recorded in association with the information on the site. More specifically, the result is recorded in association with the information on the site selected in a case of capturing the static image. After the examination has ended, the recognition result of the recognition processing for each site is displayed in a list. In the present embodiment, the results are displayed in a list using schema diagrams.

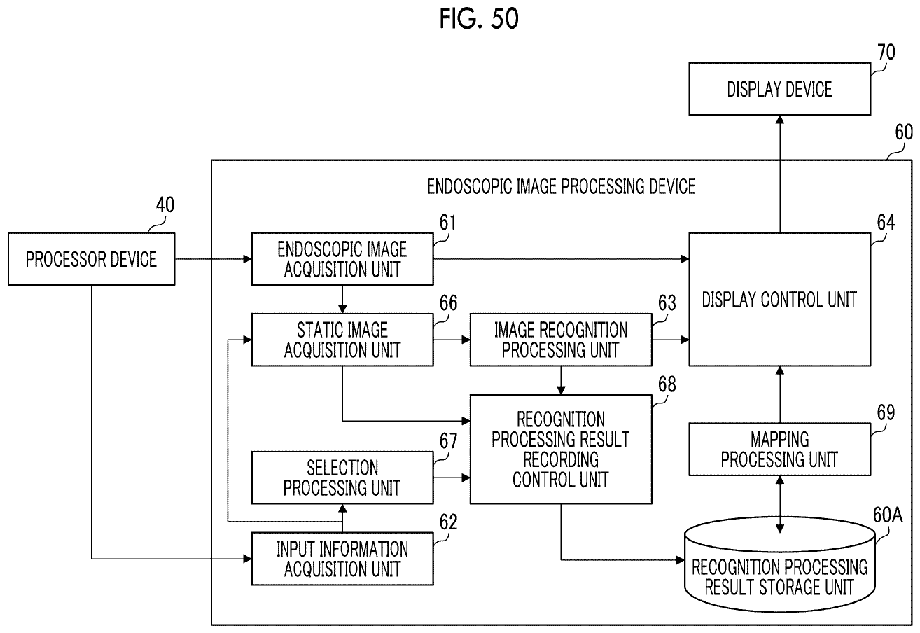

FIG. 50 is a block diagram of functions of the endoscopic image processing device for recording and outputting the result of the recognition processing.

As illustrated in the figure, for recording and outputting the result of the recognition processing, the endoscopic image processing device 60 has functions of the endoscopic image acquisition unit 61, the input information acquisition unit 62, the image recognition processing unit 63, the display control unit 64, a static image acquisition unit 66, a selection processing unit 67, a recognition processing result recording control unit 68, a mapping processing unit 69, and a recognition processing result storage unit 60A. The functions of the endoscopic image acquisition unit 61, the input information acquisition unit 62, the image recognition processing unit 63, the display control unit 64, the static image acquisition unit 66, the selection processing unit 67, the recognition processing result recording control unit 68, and the mapping processing unit 69 are realized by the processor of the endoscopic image processing device 60 executing predetermined programs. Further, the function of the recognition processing result storage unit 60A is realized by the main storage unit and/or auxiliary storage unit of the endoscopic image processing device 60.

The endoscopic image acquisition unit 61 acquires the endoscopic image from the processor device 40.

The input information acquisition unit 62 acquires information input from the input device 50 and the endoscope 20 via the processor device 40. In the acquired information, an imaging instruction for the static image and a rejection instruction for the result of the recognition processing are included. The imaging instruction for the static image is performed, for example, by a shutter button provided on the operation part 22 of the endoscope 20. The rejection instruction for the result of the recognition processing is performed by the foot switch. This point will be described later.

The static image acquisition unit 66 acquires the static image according to the imaging instruction for the static image from the user. For example, the static image acquisition unit 66 acquires, as the static image, an image of a frame displayed on the display device 70 at the time point when an instruction to capture the static image is given. The acquired static image is applied to the image recognition processing unit 63 and the recognition processing result recording control unit 68.

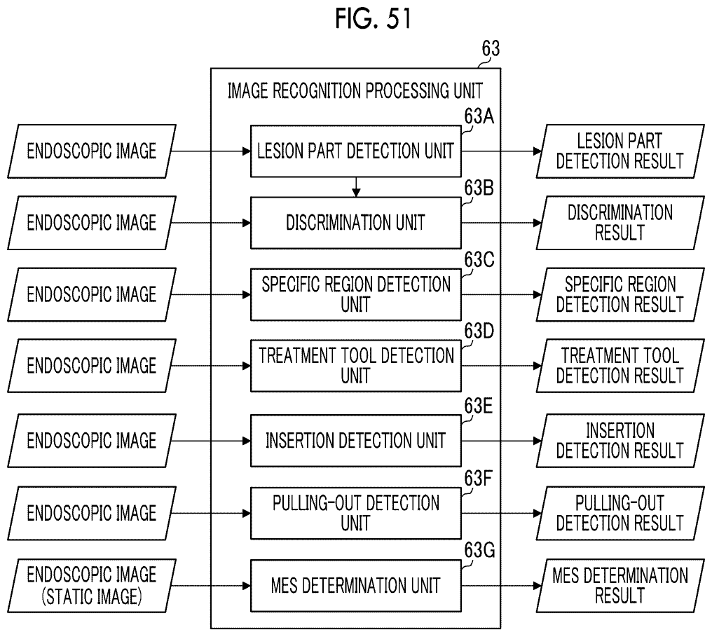

FIG. 51 is a block diagram of main functions of the image recognition processing unit.

As illustrated in the figure, the image recognition processing unit 63 of the present embodiment further includes a function of an MES determination unit 63G.

The IVIES determination unit 63G performs the image recognition on the captured static image, and determines the Mayo score (MES). That is, the static image is input, and the Mayo score is output. The MES determination unit 63G is configured by the AI or the trained model trained using a machine learning algorithm or deep learning. More specifically, the IVIES determination unit 63G is configured by the AI or the trained model trained to output the Mayo score from the static image of the endoscope. The determination result is applied to the display control unit 64 and the recognition processing result recording control unit 68.

The display control unit 64 controls display of the display device 70. As in the first embodiment described above, the display control unit 64 displays the image (endoscopic image) captured by the endoscope 20 on the display device 70 in real time. Further, predetermined information is displayed on the display device 70 according to an operation situation of the endoscope, a processing result of the image recognition of the image recognition processing unit 63, and the like. This information includes the determination result of the Mayo score. Display of the screen will be described later.

The selection processing unit 67 performs selection processing of the site and selection processing of adopting or rejecting the result of the recognition processing on the basis of the information acquired via the input information acquisition unit 62. In the present embodiment, the selection processing of the site and the selection processing of adopting or rejecting the result of the recognition processing are performed on the basis of the operation information of the foot switch.

As described above, for the selection processing of the site, processing of switching the site being selected in order is performed each time the foot switch is operated.

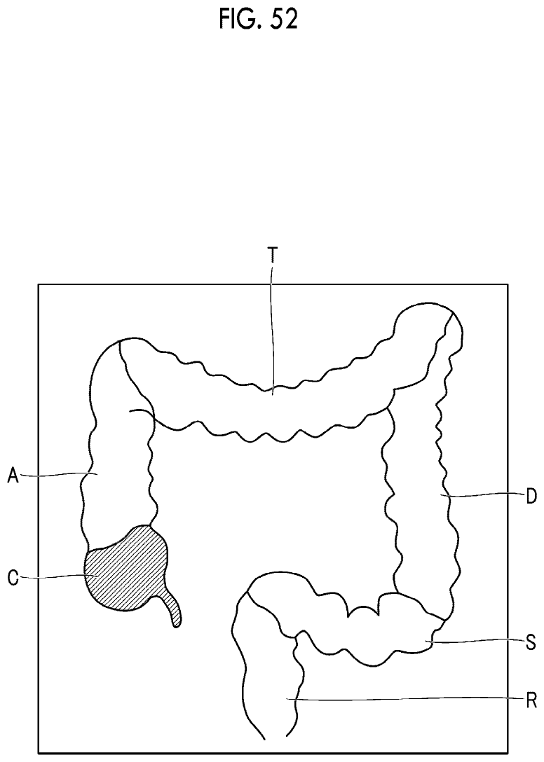

FIG. 52 is a diagram illustrating an example of the site selection box. As illustrated in the figure, in the present embodiment, the large intestine is selected from six sites. Specifically, the large intestine is selected from six sites of “Cecum” indicated by symbol C, “ASCENDING COLON” indicated by symbol A, “TRANSVERSE COLON” indicated by symbol T, “DESCENDING COLON” indicated by symbol D, “Sigmoid colon” indicated by symbol S, and “Rectum” indicated by symbol R. Therefore, in the present embodiment, each time the foot switch is operated, (1) cecum C, (2) ascending colon A, (3) transverse colon T, (4) descending colon D, (5) sigmoid colon S, and (6) rectum R are looped and switched in this order. FIG. 52 illustrates an example of the display of the site selection box in a case where the site being selected is the cecum C.

The selection processing of adopting or rejecting the result of the recognition processing, that is, the determination result of the Mayo score, is performed as follows. That is, only the rejection instruction is accepted within a certain period. In a case where there is no rejection instruction within a certain period, adoption is confirmed. The rejection instruction is performed by pressing the foot switch for a long time. In the present embodiment, in a case where the foot switch is pressed for a long time within a fixed time (time T5) from the display of the Mayo score on the screen of the display device 70, the operation is processed as rejection. On the other hand, in a case where a fixed time (time T5) has elapsed without the foot switch being pressed for a long time, adoption is confirmed. The recording of the recognition processing result (determination result of the Mayo score) is canceled by the rejection. This processing will be described below in detail.

The recognition processing result recording control unit 68 performs processing of recording information on the captured static image and on the result (determination result of the Mayo score) of the recognition processing for the static image in the recognition processing result storage unit 60A. The information on the static image and on the result of the recognition processing for the static image is recorded in association with the information on the site selected in a case where the static image is captured.

The mapping processing unit 69 performs processing of generating data indicating the result of the series of recognition processing. In the present embodiment, the data indicating the result of the series of recognition processing is generated using the schema diagram. Specifically, data (hereinafter, referred to as map data) obtained by mapping the result of the recognition processing for each site is generated using the schema diagram.

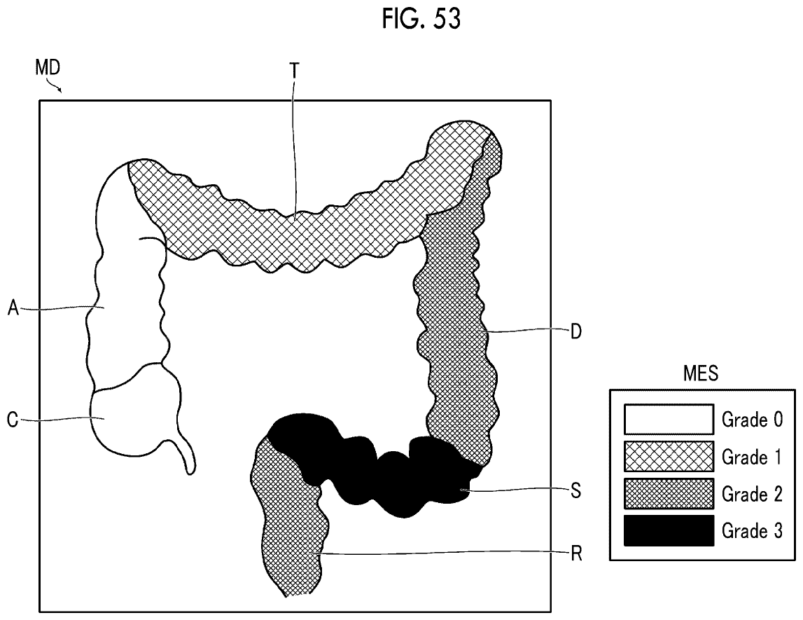

FIG. 53 is a diagram illustrating an example of map data.

As illustrated in the figure, in the present embodiment, map data MD is generated by assigning a color corresponding to the result of the recognition processing to each site on the schema diagram and mapping the recognition processing. Specifically, the map data MD is generated by assigning the color corresponding to the Mayo score (MES) to each site on the schema diagram. FIG. 53 illustrates an example of a case where the Mayo score of the cecum C is zero (Grade 0), the Mayo score of the ascending colon A is zero (Grade 0), the Mayo score of the transverse colon T is one (Grade 1), the Mayo score of the descending colon D is two (Grade 2), the Mayo score of the sigmoid colon S is three (Grade 3), and the Mayo score of the rectum R is two (Grade 2).

In this manner, by displaying the result (Mayo score) of the recognition processing of each site in color on the schema diagram, it is possible to easily understand the information on each site (severity of the ulcerative colitis in the present embodiment).

The generated map data MD is applied to the display control unit 64, and is output to the display device 70. In the present embodiment, the map data MD is an example of second information.

[Operation]

The operation of the endoscopic image processing device of the present embodiment configured as described above is as follows.

Here, only the function of recording the result of the recognition processing and the function of generating the map data on the basis of the recorded information will be described.

[Function of Recording Result of Recognition Processing]

The function of recording the result (determination result of the Mayo score) of the recognition processing is enabled in a case where the function is turned on. Hereinafter, the function of recording the determination result of the Mayo score is referred to as a Mayo score recording function. For example, ON and OFF of the Mayo score recording function are performed on a predetermined setting screen.

As described above, the Mayo score is recorded in association with the information on the site. Thus, first, the selection processing of the site in the endoscopic image processing device of the present embodiment will be described.

Selection of the site is performed in the site selection box. As in the first embodiment described above, the site selection box is displayed on the screen in a case where the ileocecum is detected from the endoscopic image. In addition, in the present embodiment, the site selection box is displayed on the screen by a manual input of the ileocecum being reached. Further, in the present embodiment, the selection processing of the site is ended by the detection of the pulling-out of the endoscope from the body cavity or by the manual input of the pulling-out.

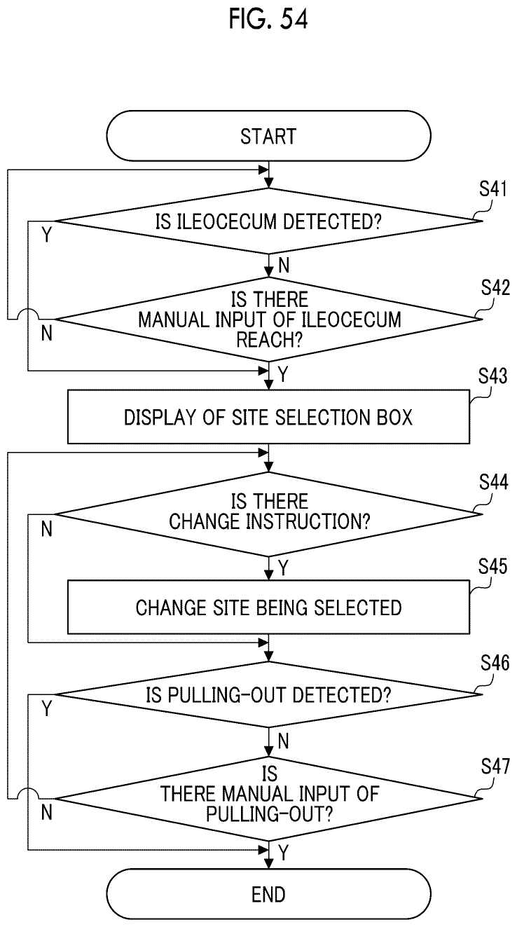

FIG. 54 is a flowchart illustrating a procedure of the selection processing of the site.

First, it is determined whether or not the ileocecum is detected (Step S41). In a case where it is determined that the ileocecum is not detected, it is determined whether or not there is a manual input of the ileocecum being reached (Step S42).

In a case where the ileocecum is detected, or in a case where there is a manual input of the ileocecum being reached, the site selection box is displayed at a predetermined position on the screen of the display device 70 (Step S43). In this case, the site selection box is displayed in a state where one site is selected in advance. In the present embodiment, the site selection box is displayed in a state where the cecum C is selected (refer to FIG. 52). Further, the site selection box is displayed by being enlarged for a fixed time, and then is displayed by being reduced to a normal size.

After the display of the site selection box is started, it is determined whether or not there is a change instruction of a site (Step S44). The change instruction of the site is performed by the foot switch. Therefore, it is determined whether or not there is a change instruction of the site by determining whether or not the foot switch is pressed.

In a case where it is determined that there is a change instruction of the site, the site being selected is changed (Step S45). The site is switched in order each time the foot switch is pressed. The information on the site being selected is held in the main storage unit, for example. The display of the site selection box is updated by the change of the site.

After the site being selected is changed, it is determined whether or not the pulling-out is detected (Step S46). Also in a case where it is determined in Step S44 that there is no change instruction of the site, it is determined whether or not the pulling-out is detected (Step S46).

In a case where it is determined that the pulling-out is not detected, it is determined whether or not there is a manual input of the pulling-out (Step S47). In a case where it is determined that there is a manual input of the pulling-out, the processing of the site selection is ended. Also in a case where it is determined in Step S46 that the pulling-out is detected, the selection processing of the site is ended.

On the other hand, in a case where it is determined that there is no manual input of the pulling-out, the processing returns to Step S44, and it is determined whether or not there is a change instruction of the site.

In this manner, the selection of the site is performed using the site selection box displayed on the screen.

Next, the recording processing of the result (determination result of the Mayo score) of the recognition processing will be described.

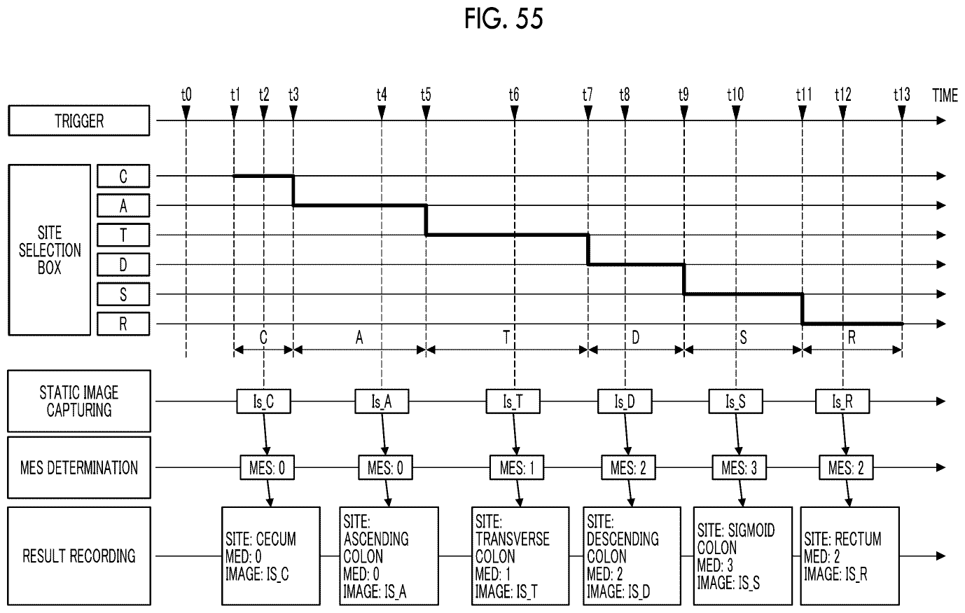

FIG. 55 is a diagram illustrating an outline of the recording processing of the Mayo score. FIG. 55 illustrates the flow of the series of recording processing from the start to the end of the examination.

It is assumed that the endoscope is inserted into the body cavity at a time point of time point to. Then, at time point t1, in a case where the ileocecum is detected from the image, or in a case where the ileocecum being reached is manually input, the site selection box is displayed on the screen of the display device 70. In this case, the site selection box is displayed in a state where the cecum C is selected. In a case of changing the site, the site being selected is switched by operating the foot switch.

While the cecum C is being selected, at time point t2, in a case where an instruction to capture a static image is given, the static image is captured. A captured static image Is_C is applied to the MES determination unit 63G, and the Mayo score is determined. The determined Mayo score (MES: 0) and the captured static image Is_C are recorded in the auxiliary storage unit in association with the information on the site (cecum C).

At time point t3, in a case where an instruction to change the site is given by operating the foot switch, the site being selected is switched from the cecum C to the ascending colon A. At the same time, the display of the site selection box is updated. That is, the display is updated such that the site being selected is the ascending colon A.

While the ascending colon A is being selected, at time point t4, in a case where an instruction to capture a static image is given, the static image is captured. A captured static image Is_A is applied to the MES determination unit 63G, and the Mayo score is determined. The determined Mayo score (MES: 0) and the captured static image Is_A are recorded in the auxiliary storage unit in association with the information on the site (ascending colon A).

At time point t5, in a case where an instruction to change the site is given by operating the foot switch, the site being selected is switched from the ascending colon A to the transverse colon T. At the same time, the display of the site selection box is updated. That is, the display is updated such that the site being selected is the transverse colon T.

While the transverse colon T is being selected, at time point t6, in a case where an instruction to capture a static image is given, the static image is captured. A captured static image Is_T is applied to the MES determination unit 63G, and the Mayo score is determined. The determined Mayo score (MES: 1) and the captured static image Is_T are recorded in the auxiliary storage unit in association with the information on the site (transverse colon T).

At time point t7, in a case where an instruction to change the site is given by operating the foot switch, the site being selected is switched from the transverse colon T to the descending colon D. At the same time, the display of the site selection box is updated. That is, the display is updated such that the site being selected is the descending colon D.

While the descending colon D is being selected, at time point t8, in a case where an instruction to capture a static image is given, the static image is captured. A captured static image Is_D is applied to the MES determination unit 63G, and the Mayo score is determined. The determined Mayo score (MES: 2) and the captured static image Is_D are recorded in the auxiliary storage unit in association with the information on the site (descending colon D).

At time point t9, in a case where an instruction to change the site is given by operating the foot switch, the site being selected is switched from the descending colon D to the sigmoid colon S. At the same time, the display of the site selection box is updated. That is, the display is updated such that the site being selected is the sigmoid colon S.

While the sigmoid colon S is being selected, at time point t10, in a case where an instruction to capture a static image is given, the static image is captured. A captured static image Is_S is applied to the MES determination unit 63G, and the Mayo score is determined. The determined Mayo score (MES: 3) and the captured static image Is_S are recorded in the auxiliary storage unit in association with the information on the site (sigmoid colon S).

At time point t11, in a case where an instruction to change the site is given by operating the foot switch, the site being selected is switched from the sigmoid colon S to the rectum R. At the same time, the display of the site selection box is updated. That is, the display is updated such that the site being selected is the rectum R.

While the rectum R is being selected, at time point t12, in a case where an instruction to capture a static image is given, the static image is captured. A captured static image Is_R is applied to the MES determination unit 63G, and the Mayo score is determined. The determined Mayo score (MES: 2) and the captured static image Is_R are recorded in the auxiliary storage unit in association with the information on the site (rectum R).

Then, at time point t13, in a case where the pulling-out is detected, or in a case where the pulling-out is manually input, the display of the site selection box disappears from the screen of the display device 70.

The examination is ended as described above. In this manner, in a case where the static image is captured during the examination, the recognition processing is performed on the captured static image by the MES determination unit 63G, and the Mayo score is determined. The determined Mayo score and the captured static image are recorded in the auxiliary storage unit in association with the information on the site being selected.

Note that the determination result of the Mayo score by the MES determination unit 63G is recorded only in a case where the user adopts the output determination result.

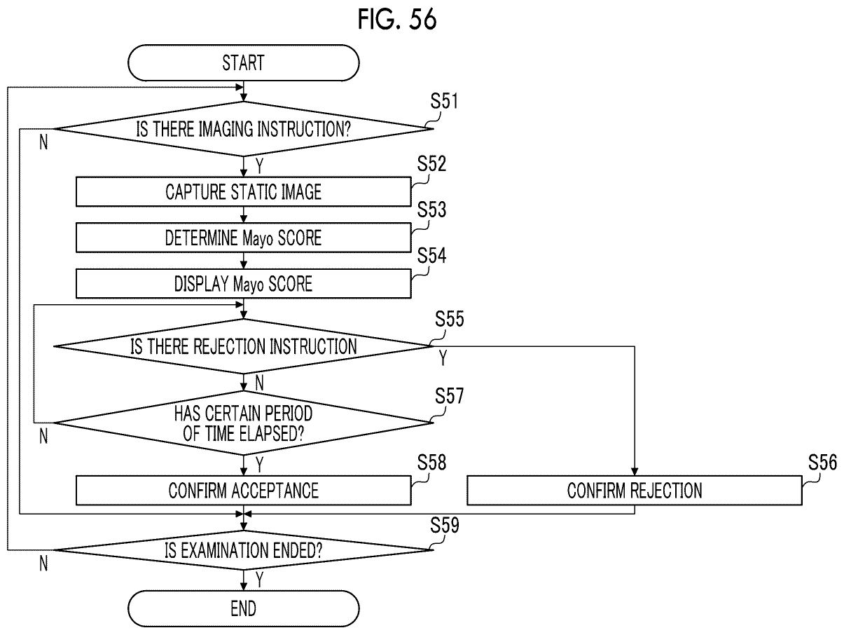

FIG. 56 is a flowchart illustrating a procedure of processing of determining the Mayo score and adopting or rejecting the results.

First, it is determined whether or not there is an imaging instruction for a static image (Step S51). In a case where it is determined that there is an imaging instruction, the static image is captured (Step S52). In a case where the static image is captured, the recognition processing is performed on the captured static image by the MES determination unit 63G, and the Mayo score is determined (Step S53). The determination result is displayed on the display device 70 for a fixed time (time T5).

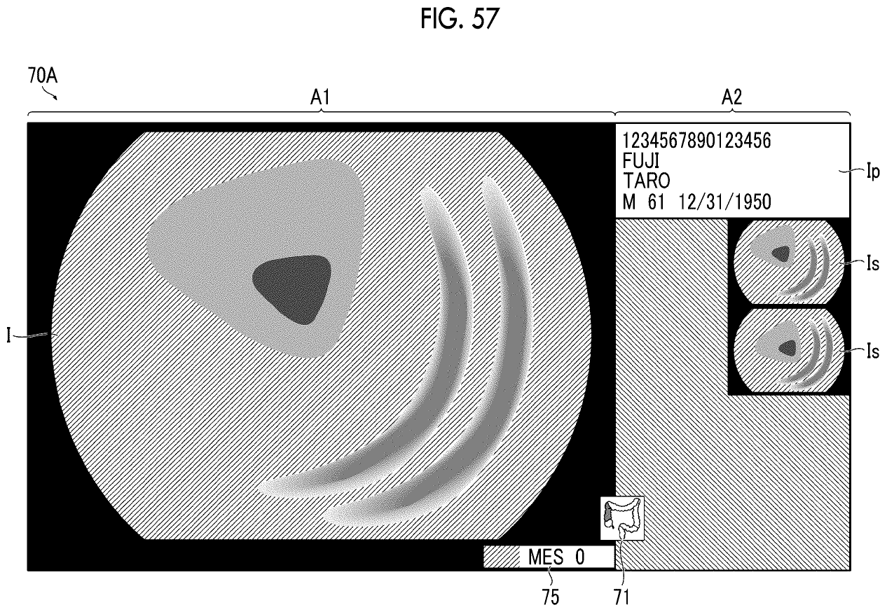

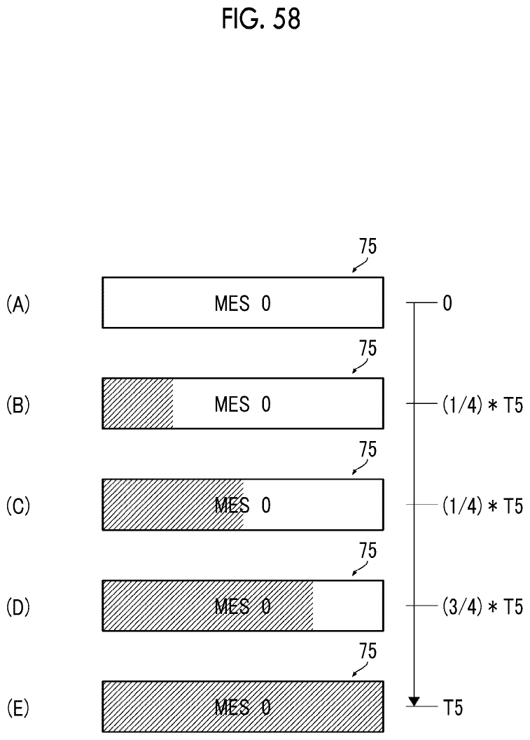

FIG. 57 is a diagram illustrating an example of display of the determination result of the Mayo score.

As illustrated in the figure, a Mayo score display box 75 is displayed at a fixed position on the screen 70A, and the determination result of the Mayo score is displayed in the Mayo score display box 75. In the example illustrated in the figure, the Mayo score display box 75 is displayed in the vicinity of the site selection box 71. In the present embodiment, the region where the Mayo score display box 75 is displayed is an example of a fourth region. Further, the Mayo score displayed in the Mayo score display box 75 is an example of first information.

The Mayo score display box 75 is displayed on the screen 70A for a fixed time (time T5). Therefore, after a fixed time has elapsed from the start of the display, the display disappears from the screen.

In the present embodiment, the Mayo score display box 75 serves as a progress bar, and the background color thereof is changed over time from the left side to the right side of the screen.

FIG. 58 is a diagram illustrating a temporal change of the display of the Mayo score display box.

(A) of FIG. 58 illustrates a display state in a case where the display has started. Further, (B) to (D) of FIG. 58 respectively illustrate a display state after a time of (1/4)*T5 has elapsed from the start of the display, a display state after a time of (2/4)*T5 has elapsed from the start of the display, and a display state after a time of (3/4)*T5 has elapsed from the start of the display. Further, (E) of FIG. 58 illustrates a display state after a fixed time (time T5) has elapsed from the start of the display. As illustrated in the figure, the background color is changed over time from the left side to the right side of the screen. In this example, the white portion indicates the remaining time. A fixed time (time T5) has elapsed at a stage where the entire background color has been switched.

In a case where the display of the Mayo score display box 75 is started, it is determined whether or not there is a rejection instruction for the determination result of the Mayo score displayed in the Mayo score display box 75 (Step S55). The rejection instruction is performed by an operation of pressing the foot switch for a long time. Further, the rejection instruction is accepted only while the determination result of the Mayo score is displayed.

In a case where it is determined that there is a rejection instruction, the rejection is confirmed (Step S56). In this case, the determination result of the Mayo score is not recorded, and only the static image is recorded in association with the information on the site.

On the other hand, in a case where it is determined that there is no rejection instruction, it is determined whether or not a fixed time (time T5) has elapsed from the start of the display of the Mayo score display box 75 (Step S57). In a case where it is determined that a fixed time has not elapsed, the processing returns to Step S55, and it is determined again whether or not there is a rejection instruction. On the other hand, in a case where it is determined that a fixed time has elapsed, the adoption is confirmed. In this case, the determination result of the Mayo score and the static image are recorded in association with the information on the site. In the present embodiment, time T5 for which the determination result of the Mayo score is displayed is an example of fourth time.

Then, it is determined whether or not the examination has ended (Step S59). In the present embodiment, it is determined whether or not the examination has ended depending on whether or not the endoscope is pulled out of the body cavity. Therefore, in a case where the pulling-out is detected, or in a case where the pulling-out is manually input, it is determined that the examination has ended.

In a case where it is determined that the examination has ended, the processing is ended. On the other hand, in a case where it is determined that the examination has continued, the processing returns to Step S51, and it is determined whether or not there is an imaging instruction for a static image.

In this manner, in the present embodiment, it is possible for the user to arbitrarily select the adoption or rejection of the determination result of the Mayo score of the MES determination unit 63G. Accordingly, it is possible to prevent unintended results from being recorded.

Further, in a case of selecting the adoption or rejection of the result, it is possible to save the user's time and effort for the selection operation of the adoption or rejection by adopting a configuration in which only the rejection instruction is accepted. Accordingly, it is possible to concentrate on the examination (observation).

[Function of Generating Map Data]

The map data MD is generated according to a generation instruction from the user after the examination has ended. For example, the generation instruction is performed on a predetermined operation screen by displaying the operation screen using a keyboard, a mouse, or the like.

In a case where an instruction to generate the map data is given, the map data MD is generated by the mapping processing unit 69. The mapping processing unit 69 generates the map data MD on the basis of the result (determination result of the Mayo score) of the series of recognition processing recorded in the auxiliary storage unit. Specifically, the map data MD is generated by assigning a color corresponding to the determined Mayo score to each site on the schema diagram (refer to FIG. 53).

For example, the map data is generated as an image in a format based on the international standard Digital Imaging and Communications in Medicine (DICOM). The generated map data MD is displayed on the display device 70 via the display control unit 64.

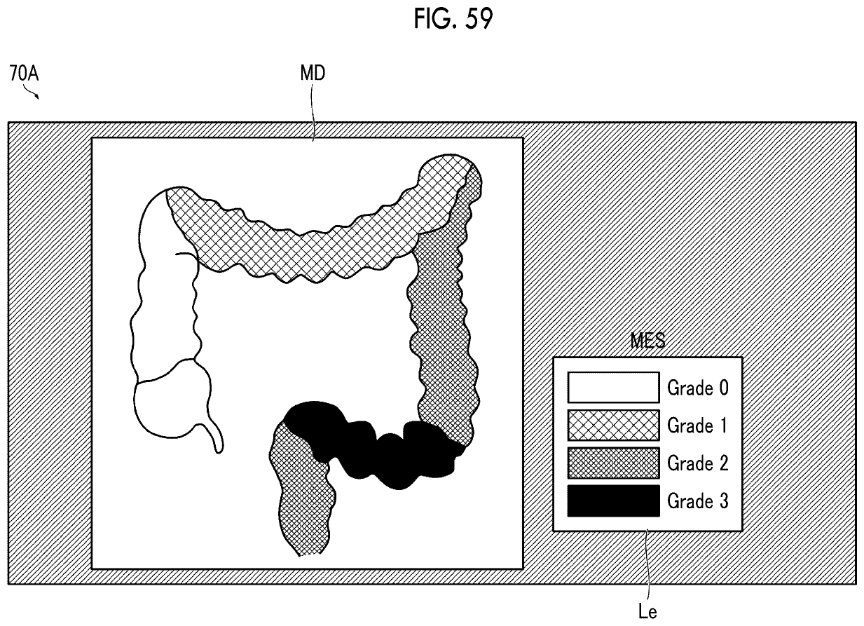

FIG. 59 is a diagram illustrating an example of the display of the map data.

As illustrated in the figure, the map data MD is displayed on the screen 70A of the display device. In the example illustrated in the figure, a legend Le is displayed at the same time.

It is possible for the user to understand the severity of the ulcerative colitis in each site at a glance by viewing the map data MD.

The map data MD is output to the endoscope information management system 100 according to the instruction from the user. The endoscope information management system 100 records the acquired map data MD in the database 120 including the examination information.

### Modification Example

[Case of Performing Recognition Processing a Plurality of Times on One Site]

There is a case where the recognition processing is performed a plurality of times on one site. In this case, all the results of the recognition processing (excluding the results of the recognition processing for rejection) are recorded in association with the information on the site. For example, in a case where, in the transverse colon T, the static image is captured a plurality of times and the Mayo score is determined a plurality of times, all the images and scores are recorded. In this case, the recognition results are recorded in chronological order so that each recognition result is distinguishable. For example, the result of each recognition processing is recorded in association with the information on the imaging date and time or the elapsed time from the start of the examination.

In a case where a plurality of Mayo scores are recorded for one site, the map data is generated as follows.

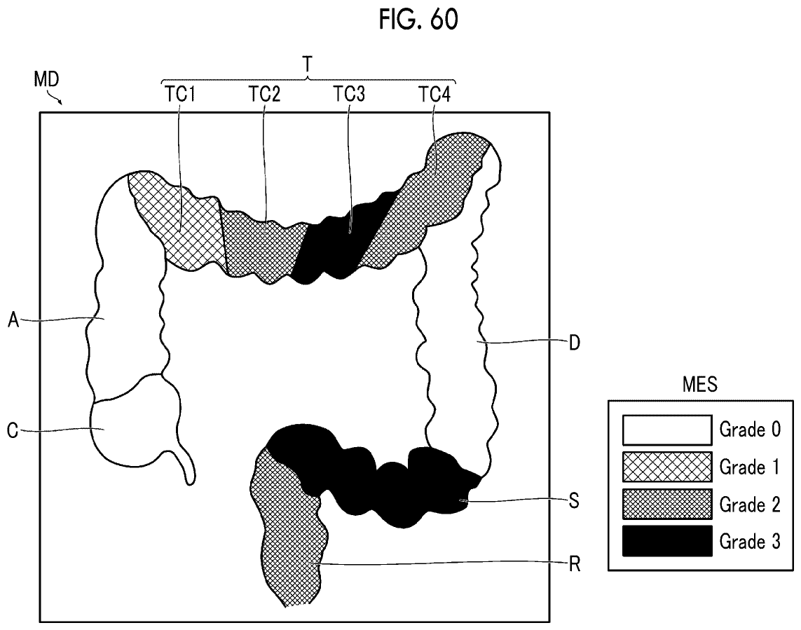

FIG. 60 is a diagram illustrating an example of the map data in a case where a plurality of Mayo scores are recorded for one site. FIG. 60 illustrates an example of a case in which four Mayo scores are associated and recorded for the transverse colon T.

As illustrated in the figure, the site for which a plurality of Mayo scores are recorded is further divided into a plurality of sites, and the results are displayed. FIG. 60 illustrates an example of a case in which four Mayo scores are recorded for the transverse colon T, and thus, the site of the transverse colon T of the schema diagram is divided into four sites along an observation direction. Sites obtained by further dividing the site divided by default (in this example, cecum C, ascending colon A, transverse colon T, descending colon D, sigmoid colon S, and rectum R) are referred to as detailed sites. In the example illustrated in FIG. 60, the transverse colon T is divided into four detailed sites TC1 to TC4. The detailed sites TC1 to TC4 are set by roughly equally dividing a target site. The sites are TC1, TC2, TC3, and TC4 from an upstream side in the observation direction (direction from the cecum to the rectum).

The Mayo scores are assigned in order from the upstream side in the observation direction in chronological order. Therefore, a first Mayo score is assigned to the detailed site TC1 in chronological order. A second Mayo score is assigned to the detailed site TC2 in chronological order. A third Mayo score is assigned to the detailed site TC3 in chronological order. A fourth Mayo score is assigned to the detailed site TC4 in chronological order.

FIG. 60 illustrates a case where, in chronological order, the first Mayo score is one (Grade 1), the second Mayo score is two (Grade 2), the third Mayo score is three (Grade 3), and the fourth Mayo score is two (Grade 2).

In this manner, in a case where a plurality of results of the recognition processing are recorded for one site, the site is divided into a plurality of sites (detailed sites), and the results are displayed. Accordingly, the results of the recognition processing can be displayed without omission. In the present embodiment, the transverse colon T is an example of a first site. Further, the four detailed sites TC1 to TC4 obtained by dividing the transverse colon T are examples of second sites.

[Modification Examples of Map Data]

In the embodiment described above, the map data is generated using the schema diagram of the hollow organ as the examination target (observation target), but the format of the map data is not limited thereto.

### (1) First Modification Example of Map Data

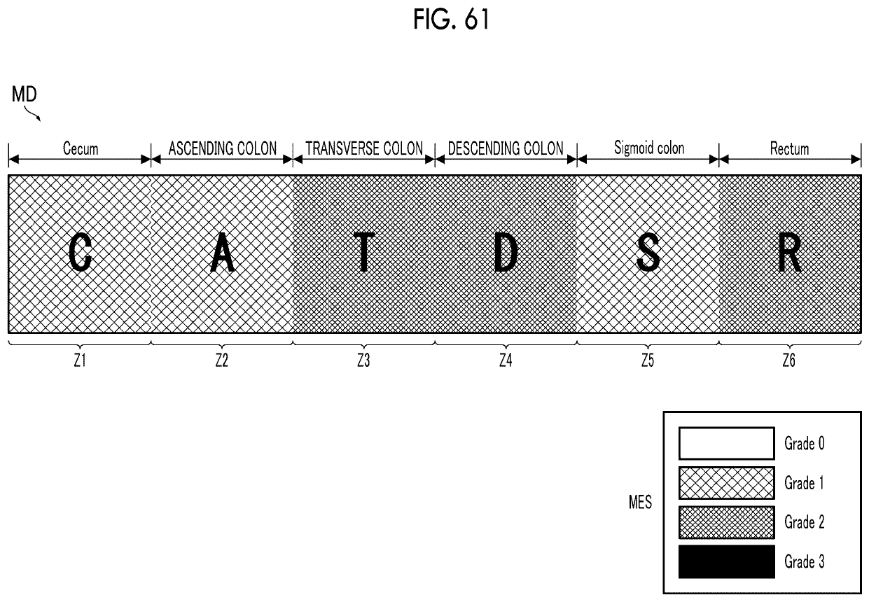

FIG. 61 is a diagram illustrating another example of the map data.

FIG. 61 illustrates an example of a case where the map data MD is generated using a strip graph. The map data MD is generated by equally dividing a rectangular frame extending in the horizontal direction into a plurality of regions according to the number of sites. For example, in a case where the number of sites set in the hollow organ as the examination target is six, the frame is equally divided into six regions along the horizontal direction. Each site is assigned to each divided region. The sites are assigned in order from a right region toward a left region of the frame along the observation direction.

FIG. 61 is an example of a case in which the examination target is the large intestine, and illustrates an example of a case where the large intestine is divided into six sites (cecum C, ascending colon A, transverse colon T, descending colon D, sigmoid colon S, and rectum R).

The cecum C is assigned to a first divided region Z1. The ascending colon A is assigned to a second divided region Z2. The transverse colon T is assigned to a third divided region Z3. The descending colon D is assigned to a fourth divided region Z4. The sigmoid colon S is assigned to a fifth divided region Z5. The rectum R is assigned to a sixth divided region Z6.

Therefore, the Mayo score of the cecum C is displayed in the first divided region Z1. The Mayo score of the ascending colon A is displayed in the second divided region Z2. The Mayo score of the transverse colon T is displayed in the third divided region Z3. The Mayo score of the descending colon D is displayed in the fourth divided region Z4. The Mayo score of the sigmoid colon S is displayed in the fifth divided region Z5. The Mayo score of the rectum R is displayed in the sixth divided region Z6.

The Mayo score is displayed in a color corresponding to the score (Grade). FIG. 61 illustrates an example of a case where the Mayo score of the cecum C is one (Grade 1), the Mayo score of the ascending colon A is one (Grade 1), the Mayo score of the transverse colon T is two (Grade 2), the Mayo score of the descending colon D is two (Grade 2), the Mayo score of the sigmoid colon S is one (Grade 1), and the Mayo score of the rectum R is two (Grade 2).

A symbol indicating the assigned site is displayed in each of the divided regions Z1 to Z6. In the example illustrated in FIG. 61, the initials of the assigned sites are displayed. Therefore, a symbol “C” indicating that the cecum is assigned is displayed in the first divided region Z1. A symbol “A” indicating that the ascending colon is assigned is displayed in the second divided region Z2. A symbol “T” indicating that the transverse colon is assigned is displayed in the third divided region Z3. A symbol “D” indicating that the descending colon is assigned is displayed in the fourth divided region Z4. A symbol “S” indicating that the sigmoid colon is assigned is displayed in the fifth divided region Z5. A symbol “R” indicating that the rectum is assigned is displayed in the sixth divided region Z6.

### (2) Second Modification Example of Map Data

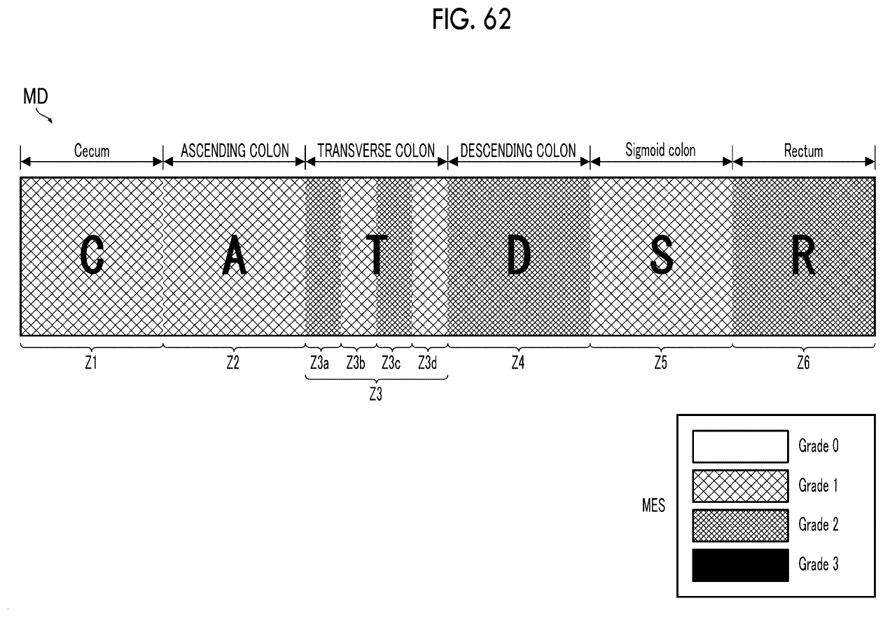

FIG. 62 is a diagram illustrating another example of the map data.

FIG. 62 illustrates an example of a case where a plurality of recognition processing results are recorded for one site in the map data with the form illustrated in FIG. 61.

FIG. 62 illustrates an example of a case where four Mayo scores are associated and recorded for the transverse colon T. In this case, the region where the transverse colon T is assigned is further divided, and the results are displayed. Since the region where the transverse colon T is assigned is the third divided region Z3, the third divided region Z3 is further divided. In this example, the region is divided into four. The division is performed along a longitudinal direction of the frame, and the target region is equally divided.

The region obtained by further dividing the divided region is referred to as a detailed divided region. In the example illustrated in FIG. 62, the third divided region Z3 is divided into four detailed divided regions Z3a to Z3d.

The Mayo scores are assigned in order from the upstream side in the observation direction in chronological order. Therefore, a first Mayo score is assigned to the detailed divided region Z3a in chronological order. A second Mayo score is assigned to the detailed divided region Z3b in chronological order. A third Mayo score is assigned to the detailed divided region Z3c in chronological order. A fourth Mayo score is assigned to the detailed divided region Z3d in chronological order.

FIG. 62 illustrates a case where, in chronological order, the first Mayo score is two (Grade 2), the second Mayo score is one (Grade 1), the third Mayo score is two (Grade 2), and the fourth Mayo score is one (Grade 1).

### (3) Third Modification Example of Map Data

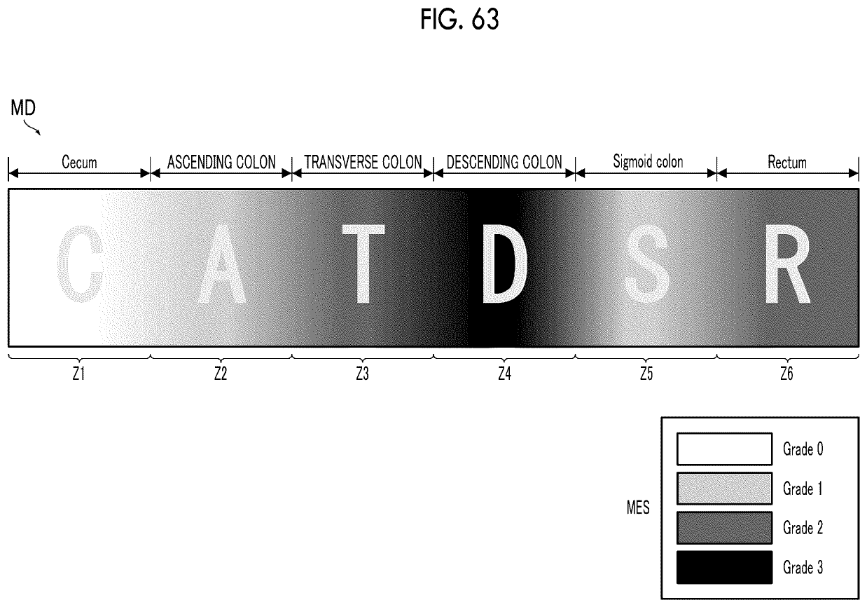

FIG. 63 is a diagram illustrating another example of the map data.

As illustrated in the figure, the map data MD in this example is generated by performing gradation processing on a boundary of each site. That is, the map data is generated such that the color is expressed to be gradually changed in the boundaries of the divided regions indicating respective sites.

FIG. 63 illustrates an example of a case where the Mayo score of the cecum C is zero (Grade 0), the Mayo score of the ascending colon A is one (Grade 1), the Mayo score of the transverse colon T is two (Grade 2), the Mayo score of the descending colon D is three (Grade 3), the Mayo score of the sigmoid colon S is one (Grade 1), and the Mayo score of the rectum R is two (Grade 2).

In this manner, for the map data, a site and a result of the recognition processing associated with each site need only be understood, and the display format or the like thereof is not particularly limited.

Further, in each example described above, a format representing the result of the recognition processing using colors is adopted, but a format representing the result using densities may be adopted. Further, a format representing the result using a design, a pattern, or the like may be adopted.

[Presentation of Map Data]

As described above, the map data MD is output to the endoscope information management system 100 according to the instruction from the user, and is recorded as the examination information.

The endoscope information management system 100 can have a function of presenting the map data to the user, as a function of supporting the diagnosis. In this case, it is preferable to present the map data in a format to be compared with past data.

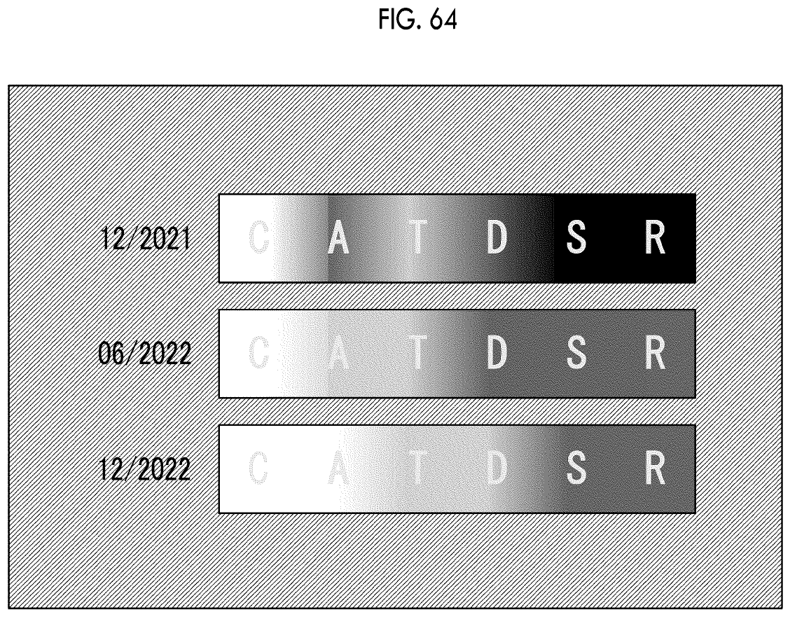

FIG. 64 is a diagram illustrating an example of the presentation of the map data.

For example, in response to a request from the user terminal 200 or the like, the endoscope information management system 100 displays the map data of the corresponding patient (subject) on the screen of the user terminal 200. In this case, in a case where a plurality of pieces of map data are present, the map data are arranged in chronological order and displayed in response to an instruction from the user. FIG. 64 illustrates an example of a case where the map data are arranged and displayed in chronological order from top to bottom of the screen.

In this manner, the diagnosis or the like can be facilitated by displaying the map data in a format to be compared with data of examinations performed in the past.

[Generation of Map Data]

In the embodiment described above, a configuration is adopted in which the map data is generated after the examination has ended, but a configuration can be adopted in which the map data is generated during the examination. For example, a configuration can be adopted in which the map data is generated at a timing when the site being selected is switched. In this case, for example, at a timing when the site is switched, the map data for the site before switching is generated, and the map data is updated. Further, in a case where the map data is generated during the examination in this manner, the generated map data may be displayed on the screen during the examination.

[Recognition Processing]

In the embodiment described above, a case where the Mayo score is determined from the static image of the endoscope and is recorded in association with the information on the site is exemplified, but the information to be recorded in association with the information on the site is not limited thereto. A configuration in which other recognition processing results are recorded can be adopted.

Further, similar to the Mayo score, for outputting scores as the recognition results, it is preferable to generate and present the map data.

Further, in the embodiment described above, a configuration is adopted in which the Mayo score is determined from the static image, but a configuration can be adopted in which the Mayo score is determined from a video. That is, a configuration can be adopted in which the recognition processing is performed on the image of each frame of the video.

### Other Embodiments

[Application to Other Medical Images]

In the embodiment described above, the image captured using a flexible endoscope (electronic endoscope) is used as the processing target image, but the application of the present invention is not limited thereto, and the present invention can be applied to a case where a medical image captured by other modalities such as an ultrasound diagnostic apparatus, X-ray equipment, digital mammography, a computed tomography (CT) device, and a magnetic resonance imaging (MM) device are used as the processing target. Further, the present invention can be applied to a case where an image captured by a rigid endoscope is used as a processing target.

[Hardware Configuration]

Further, the functions of the processor device 40 and of the endoscopic image processing device 60 in the endoscope system 10 are realized by various processors. Similarly, the functions of the endoscope information management device 110 in the endoscope information management system 100 can be realized by various processors.

The various processors include a CPU and/or a graphics processing unit (GPU) as a general-purpose processor executing a program and functioning as various processing units, a programmable logic device (PLD) as a processor of which the circuit configuration can be changed after manufacture, such as a field-programmable gate array (FPGA), and a dedicated electrical circuit as a processor having a circuit configuration designed exclusively for executing specific processing such as an application-specific integrated circuit (ASIC). The program is synonymous with software.

One processing unit may be configured by one processor among these various processors, or may be configured by two or more same or different kinds of processors. For example, one processing unit may be configured by a plurality of FPGAs, or by a combination of a CPU and an FPGA. Further, a plurality of processing units may be configured by one processor. As an example where a plurality of processing units are configured by one processor, first, there is a form where one processor is configured by a combination of one or more CPUs and software as typified by a computer used in a client, a server, or the like, and this processor functions as a plurality of processing units. Second, there is a form where a processor fulfilling the functions of the entire system including a plurality of processing units via one integrated circuit (IC) chip as typified by a system-on-chip (SoC) or the like is used. In this manner, various processing units are configured by using one or more of the above-described various processors as hardware structures.

Further, in the embodiment described above, the processor device 40 and the endoscopic image processing device 60 constituting the endoscope system 10 are separately configured, but the processor device 40 may have the function of the endoscopic image processing device 60. That is, the processor device 40 and the endoscopic image processing device 60 can be integrated. Similarly, the light source device 30 and the processor device 40 can be integrated.

[Examination Target]

In the embodiment described above, a case where the large intestine is examined is exemplified, but the application of the present invention is not limited thereto. The present invention can be similarly applied to a case where other hollow organs are examined. For example, the present invention can be similarly applied to a case where a stomach, a small intestine, or the like is examined.

[Treatment Tool]

In the embodiment described above, biopsy forceps and snares are exemplified as the treatment tool, but the treatment tool that can be used in the endoscope is not limited thereto. Treatment tools can be used as appropriate depending on the hollow organ as the examination target, the content of the treatment, and the like.

[Additional Remarks]

The following additional remarks are further disclosed with respect to the embodiments described above.

(Additional Remark 1)

An information processing apparatus including:
a first processor,
in which the first processor is configured to
   acquire an image captured using an endoscope,
   display the acquired image in a first region on a screen of a first display unit,
   display a plurality of sites of a hollow organ as an observation target in a second region on the screen of the first display unit, and
   accept selection of one site from among the plurality of sites.

(Additional Remark 2)

The information processing apparatus described in Additional Remark 1,
in which the first processor is configured to
   detect a specific region of the hollow organ from the acquired image, and
   display the plurality of sites in the first region in a case where the specific region is detected.

(Additional Remark 3)

The information processing apparatus described in Additional Remark 2,
in which the first processor displays the plurality of sites in the second region in a state where a site to which the detected specific region belongs is selected in advance from among the plurality of sites.

(Additional Remark 4)

The information processing apparatus described in Additional Remark 1,
in which, in a case where a display instruction of the plurality of sites is accepted, the first processor displays the plurality of sites in the first region in a state where one site is selected in advance from among the plurality of sites.

(Additional Remark 5)

The information processing apparatus described in any one of Additional Remarks 1 to 4,
in which the first processor displays the plurality of sites in the second region using a schema diagram.

(Additional Remark 6)

The information processing apparatus described in Additional Remark 5,
in which the first processor displays the site being selected such that the site being selected is distinguishable from the other sites, in the schema diagram displayed in the second region.

(Additional Remark 7)

The information processing apparatus described in any one of Additional Remarks 1 to 6,
in which the second region is set in a vicinity of a position where a treatment tool appears within the image displayed in the first region.

(Additional Remark 8)

The information processing apparatus described in Additional Remark 7,
in which the first processor displays the second region in an emphasized manner during a first time in a case where selection of the site is accepted.

(Additional Remark 9)

The information processing apparatus described in any one of Additional Remarks 1 to 8,
in which the first processor continuously accepts selection of the site after display of the plurality of sites is started.

(Additional Remark 10)

The information processing apparatus described in any one of Additional Remarks 1 to 9,
in which the first processor is configured to
   detect a plurality of specific regions from the acquired image, and
   execute processing of prompting selection of the site in a case where at least one of the plurality of specific regions is detected.

(Additional Remark 11)

The information processing apparatus described in any one of Additional Remarks 1 to 10,
in which the first processor is configured to
   detect a specific detection target from the acquired image, and
   execute processing of prompting selection of the site in a case where the detection target is detected.

(Additional Remark 12)

The information processing apparatus described in Additional Remark 11,
in which the detection target is at least one of a lesion part or a treatment tool.

(Additional Remark 13)

The information processing apparatus described in Additional Remark 12,
in which the first processor stops acceptance of the selection of the site during a second time after the detection target is detected.

(Additional Remark 14)

The information processing apparatus described in any one of Additional Remarks 11 to 13,
in which the first processor records information on the detection target in association with information on the selected site.

(Additional Remark 15)

The information processing apparatus described in any one of Additional Remarks 9 to 14,
in which the first processor displays the second region in an emphasized manner as processing of prompting selection of the site.

(Additional Remark 16)

The information processing apparatus described in any one of Additional Remarks 1 to 15,
in which the first processor is configured to
   detect a treatment tool from the acquired image,
   choose a plurality of treatment names corresponding to the detected treatment tool,
   display the plurality of chosen treatment names in a third region on the screen of the first display unit,
   accept selection of one treatment name from among the plurality of treatment names from start of the display until a third time elapses, and
   stop acceptance of selection of the site while selection of the treatment name is accepted.

(Additional Remark 17)

The information processing apparatus described in Additional Remark 16,
in which the first processor records information on the selected treatment name in association with information on the selected site.

(Additional Remark 18)

The information processing apparatus described in any one of Additional Remarks 1 to 17,
in which the first processor is configured to
   perform recognition processing on the acquired image, and
   record a result of the recognition processing in association with information on the selected site.

(Additional Remark 19)

The information processing apparatus described in Additional Remark 18,
in which the first processor performs the recognition processing on the image captured as a static image.

(Additional Remark 20)

The information processing apparatus described in Additional Remark 19,
in which the first processor displays first information indicating the result of the recognition processing in a fourth region on the screen of the first display unit.

(Additional Remark 21)

The information processing apparatus described in Additional Remark 20,
in which the first processor is configured to
   accept adoption or rejection of the result of the recognition processing indicated by the first information, and
   record the result of the recognition processing in a case where the result of the recognition processing is adopted.

(Additional Remark 22)

The information processing apparatus described in Additional Remark 21,
in which the first processor accepts only a rejection instruction, and confirms adoption in a case where the rejection instruction is not accepted from start of the display of the first information until a fourth time elapses.

(Additional Remark 23)

The information processing apparatus described in any one of Additional Remarks 18 to 22,
in which the first processor is configured to
   generate second information indicating a result of the recognition processing for each of the sites, and
   display the second information on the first display unit.

(Additional Remark 24)

The information processing apparatus described in Additional Remark 23,
in which the first processor is configured to
   divide a first site for which a plurality of the results of the recognition processing are recorded, among the plurality of sites, into a plurality of second sites, and
   generate the second information indicating the result of the recognition processing for each of the second sites, regarding the first site.

(Additional Remark 25)

The information processing apparatus described in Additional Remark 24,
in which the first processor is configured to
   set the second sites by equally dividing the first site, and
   generate the second information by assigning the results of the recognition processing to the second sites in chronological order along an observation direction.

(Additional Remark 26)

The information processing apparatus described in any one of Additional Remarks 23 to 25,
in which the first processor generates the second information using a schema diagram.

(Additional Remark 27)

The information processing apparatus described in any one of Additional Remarks 23 to 25,
in which the first processor generates the second information using a strip graph divided into a plurality of regions.

(Additional Remark 28)

The information processing apparatus described in any one of Additional Remarks 23 to 27,
in which the first processor generates the second information indicating the result of the recognition processing using a color or a density.

(Additional Remark 29)

The information processing apparatus described in any one of Additional Remarks 23 to 28,
in which the first processor determines severity of ulcerative colitis using the recognition processing.

(Additional Remark 30)

The information processing apparatus described in Additional Remark 29,
in which the first processor determines the severity of the ulcerative colitis using Mayo Endoscopic Subscore.

(Additional Remark 31)

The information processing apparatus described in any one of Additional Remarks 1 to 30,
in which the first processor accepts selection of the site after insertion of the endoscope is detected or after insertion of the endoscope is confirmed by a user input.

(Additional Remark 32)

The information processing apparatus described in any one of Additional Remarks 1 to 31,
in which the first processor accepts selection of the site until pulling-out of the endoscope is detected or until pulling-out of the endoscope is confirmed by a user input.

(Additional Remark 33)

The information processing apparatus described in any one of Additional Remarks 1 to 15,
in which the first processor is configured to
   detect a treatment tool from the acquired image,
   display a plurality of options regarding a treatment target in a fifth region on the screen of the first display unit in a case where the treatment tool is detected from the image, and
   accept selection of one of the plurality of options displayed in the fifth region.

(Additional Remark 34)

The information processing apparatus described in Additional Remark 33,
in which the plurality of options regarding the treatment target are a plurality of options for a detailed site or a size of the treatment target.

(Additional Remark 35)

The information processing apparatus described in any one of Additional Remarks 1 to 15,
in which the first processor is configured to
   display a plurality of options regarding a region of interest in a fifth region on the screen of the first display unit in a case where a static image to be used in a report is acquired, and
   accept selection of one of the plurality of options displayed in the fifth region.

(Additional Remark 36)

The information processing apparatus described in Additional Remark 35,
in which the plurality of options regarding the region of interest are a plurality of options for a detailed site or a size of the region of interest.

(Additional Remark 37)

The information processing apparatus described in any one of Additional Remarks 1 to 36,
in which the first processor records a captured static image in association with information on the selected site and/or information on the selected treatment name.

(Additional Remark 38)

The information processing apparatus described in Additional Remark 37,
in which the first processor records, as a candidate for an image to be used in a report or a diagnosis, the captured static image in association with the information on the selected site and/or information on the selected treatment name.

(Additional Remark 39)

The information processing apparatus described in Additional Remark 38,
in which the first processor acquires, as the candidate for the image to be used in the report or the diagnosis, a most recent static image in terms of time among static images captured before a time point when selection of the site is accepted, or an oldest static image in terms of time among static images captured after the time point when the selection of the site is accepted.

(Additional Remark 40)

A report creation support device that supports creation of a report, including:
a second processor,
in which the second processor is configured to
   display a report creation screen with at least an input field for a site, on a second display unit,
   acquire information on the site selected in the information processing apparatus described in any one of Additional Remarks 1 to 39,
   automatically input the acquired information on the site to the input field for the site, and
   accept correction of the automatically input information of the input field for the site.

(Additional Remark 41)

The report creation support device described in Additional Remark 40,
in which the second processor displays the input field for the site such that the input field for the site is distinguishable from other input fields on the report creation screen.

(Additional Remark 42)

A report creation support device that supports creation of a report, including:
a second processor,
in which the second processor is configured to
   display a report creation screen with at least input fields for a site and a static image, on a second display unit,
   acquire information on the site and the static image selected in the information processing apparatus described in any one of Additional Remarks 37 to 39,
   automatically input the acquired information on the site to the input field for the site,
   automatically input the acquired static image to the input field for the static image, and
   accept correction of the automatically input information of the input field for the site and the automatically input static image of the input field for the static image.

(Additional Remark 43)

An endoscope system including:
an endoscope;
the information processing apparatus described in any one of Additional Remarks 1 to 39; and
an input device.

(Additional Remark 44)

An information processing method including:
a step of acquiring an image captured using an endoscope;
a step of displaying the acquired image in a first region on a screen of a first display unit;
a step of detecting a specific region in a hollow organ from the acquired image;
a step of displaying a plurality of sites constituting the hollow organ to which the detected specific region belongs, in a second region on the screen of the first display unit; and
a step of accepting selection of one site from among the plurality of sites.

(Additional Remark 45)

An information processing apparatus including:
a first processor,
in which the first processor is configured to
   acquire an image captured using an endoscope,
   display the acquired image in a first region on a screen of a first display unit,
   display a plurality of sites of a hollow organ as an observation target in a second region on the screen of the first display unit,
   accept selection of one site from among the plurality of sites,
   detect a treatment tool from the acquired image,
   choose a plurality of treatment names corresponding to the detected treatment tool,
   display the plurality of chosen treatment names in a third region on the screen of the first display unit, and
   accept selection of one treatment name from among the plurality of treatment names from start of the display until a third time elapses.

(Additional Remark 46)

The information processing apparatus described in Additional Remark 45,
in which the first processor records a captured static image in association with information on the selected treatment name and/or information on the selected site.

(Additional Remark 47)

The information processing apparatus described in Additional Remark 46,
in which the first processor records, as a candidate for an image to be used in a report or a diagnosis, the static image captured during treatment in association with information on the selected treatment name and/or information on the selected site.

(Additional Remark 48)

The information processing apparatus described in Additional Remark 47,
in which the first processor acquires, as the candidate for the image to be used in the report or the diagnosis, a most recent static image in terms of time among static images captured before a time point when selection of the treatment name is accepted, or an oldest static image in terms of time among static images captured after the time point when the selection of the treatment name is accepted.

(Additional Remark 49)

A report creation support device that supports creation of a report, including:
a second processor,
in which the second processor is configured to
   display a report creation screen with at least input fields for a treatment name, a site, and a static image, on a second display unit,
   acquire information on the treatment name, information on the site, and the static image selected in the information processing apparatus described in any one of Additional Remarks 45 to 48,
   automatically input the acquired information on the treatment name to the input field for the treatment name,
   automatically input the acquired information on the site to the input field for the site,
   automatically input the acquired static image to the input field for the static image, and
   accept correction of the automatically input information of the input fields for the treatment name and the site, and the automatically input static image of the input field for static image.

### EXPLANATION OF REFERENCES

1: endoscopic image diagnosis support system
10: endoscope system
20: endoscope
21: insertion part of endoscope
21A: distal end portion of insertion part
21B: bendable portion of insertion part
21C: soft portion of insertion part
21a: observation window of distal end portion
21b: illumination window of distal end portion
21c: air/water supply nozzle of distal end portion
21d: forceps outlet of distal end portion
22: operation part of endoscope
22A: angle knob of operation part
22B: air/water supply button of operation part
22C: suction button of operation part
22D: forceps insertion port of operation part
23: connection part of endoscope
23A: cord of connection part
23B: light guide connector of connection part
23C: video connector of connection part
30: light source device
40: processor device
41: endoscope control unit of processor device
42: light source control unit of processor device
43: image processing unit of processor device
44: input control unit of processor device
45: output control unit of processor device
50: input device
60: endoscopic image processing device
60A: recognition processing result storage unit
61: endoscopic image acquisition unit of endoscopic image processing device
62: input information acquisition unit of endoscopic image processing device
63: image recognition processing unit of endoscopic image processing device
63A: lesion part detection unit of image recognition processing unit
63B: discrimination unit of image recognition processing unit
63C: specific region detection unit of image recognition processing unit
63D: treatment tool detection unit of image recognition processing unit
63E: insertion detection unit
63F: pulling-out detection unit
63G: MES determination unit
64: display control unit of endoscopic image processing device
65: examination information output control unit of endoscopic image processing device
66: static image acquisition unit
67: selection processing unit
68: recognition processing result recording control unit
69: mapping processing unit
70: display device
70A: screen of display device
71: site selection box displayed on screen of display device
72: treatment tool detection icon displayed on screen of display device
73: treatment name selection box displayed on screen of display device
74: progress bar displayed on screen of display device
75: Mayo score display box
75A: outside-body icon
75B: inside-body icon
76A: insertion detection icon
76B: ileocecum reached icon
76C: pulling-out detection icon
77A: progress bar displayed on screen of display device
77B: progress bar displayed on screen of display device
77C: progress bar displayed on screen of display device
80: treatment tool
90: detailed site selection box
91: countdown timer
92: size selection box
93: audio input icon
100: endoscope information management system
110: endoscope information management device
111: examination information acquisition unit of endoscope information management device
112: examination information recording control unit of endoscope information management device
113: information output control unit of endoscope information management device
114: report creation support unit of endoscope information management device
114A: report creation screen generation unit of report creation support unit
114B: automatic input unit of report creation support unit
114C: report generation unit of report creation support unit
120: database
130: selection screen
131: captured image display region of selection screen
132: detection list display region of selection screen
132A: card displayed in detection list display region
133: merge processing region of selection screen
140: detailed input screen
140A: input field for endoscopic image (static image)
140B1: input field for information on site
140B2: input field for information on site
140B3: input field for information on site
140C1: input field for information on diagnosis result
140C2: input field for information on diagnosis result
140C3: input field for information on diagnosis result
140D: input field for information on treatment name
140E: input field for information on size of lesion
140F: input field for information on classification by naked eye
140G: input field for information on hemostatic method
140H: input field for information on specimen number
140I: input field for information on JNET classification
140J: input field for other information
200: user terminal
A1: main display region of screen during examination
A2: secondary display region of screen during examination
A3: discrimination result display region of screen during examination
Ar: forceps direction
F: frame surrounding lesion region in endoscopic image
I: endoscopic image
Ip: information regarding patient
Is: static image
Is_A: static image
Is_C: static image
Is_D: static image
Is_R: static image
Is_S: static image
Is_T: static image
MD: map data
P: lesion part
C: cecum
A: ascending colon
T: transverse colon
TC1: site obtained by further dividing transverse colon (detailed site)
TC2: site obtained by further dividing transverse colon (detailed site)
TC3: site obtained by further dividing transverse colon (detailed site)
TC4: site obtained by further dividing transverse colon (detailed site)
D: descending colon
S: sigmoid colon
R: rectum
Sc: schema diagram
Z1: divided region of map data (first divided region)
Z2: divided region of map data (second divided region)
Z3: divided region of map data (third divided region)
Z3a: region obtained by further dividing third divided region (detailed divided region)
Z3b: region obtained by further dividing third divided region (detailed divided region)
Z3c: region obtained by further dividing third divided region (detailed divided region)
Z3d: region obtained by further dividing third divided region (detailed divided region)
Z4: divided region of map data (fourth divided region)
Z5: divided region of map data (fifth divided region)
Z6: divided region of map data (sixth divided region)
S1 to S11: procedure of processing of accepting input of site
S21 to S37: procedure of processing of accepting input of treatment name
S41 to S47: procedure of selection processing of site
S51 to S59: procedure of processing of determining Mayo score and adopting or rejecting results

What is claimed is:

1. An information processing apparatus comprising:
a first processor,
wherein the first processor is configured to
acquire an image captured using an endoscope,
display the acquired image in a first region on a screen of a first display unit,
display a plurality of sites of a hollow organ as an observation target in a second region on the screen of the first display unit, and
accept selection of one site from among the plurality of sites.

2. The information processing apparatus according to claim 1,
wherein the first processor is configured to
detect a specific region of the hollow organ from the acquired image, and
display the plurality of sites in the first region in a case where the specific region is detected.

3. The information processing apparatus according to claim 2,
wherein the first processor displays the plurality of sites in the second region in a state where a site to which the detected specific region belongs is selected in advance from among the plurality of sites.

4. The information processing apparatus according to claim 1,
wherein, in a case where a display instruction of the plurality of sites is accepted, the first processor displays the plurality of sites in the first region in a state where one site is selected in advance from among the plurality of sites.

5. The information processing apparatus according to claim 1,
wherein the first processor displays the plurality of sites in the second region using a schema diagram.

6. The information processing apparatus according to claim 5,
wherein the first processor displays the site being selected such that the site being selected is distinguishable from the other sites, in the schema diagram displayed in the second region.

7. The information processing apparatus according to claim 1,
wherein the second region is set in a vicinity of a position where a treatment tool appears within the image displayed in the first region.

8. The information processing apparatus according to claim 7,
wherein the first processor displays the second region in an emphasized manner during a first time in a case where selection of the site is accepted.

9. The information processing apparatus according to claim 1,
wherein the first processor continuously accepts selection of the site after display of the plurality of sites is started.

10. The information processing apparatus according to claim 1,
wherein the first processor is configured to
detect a plurality of specific regions from the acquired image, and
execute processing of prompting selection of the site in a case where at least one of the plurality of specific regions is detected.

11. The information processing apparatus according to claim 1,
wherein the first processor is configured to
detect a specific detection target from the acquired image, and
execute processing of prompting selection of the site in a case where the detection target is detected.

12. The information processing apparatus according to claim 11,
wherein the detection target is at least one of a lesion part or a treatment tool.

13. The information processing apparatus according to claim 12,
wherein the first processor stops acceptance of the selection of the site during a second time after the detection target is detected.

14. The information processing apparatus according to claim 11,
wherein the first processor records information on the detection target in association with information on the selected site.

15. The information processing apparatus according to claim 9,
wherein the first processor displays the second region in an emphasized manner as processing of prompting selection of the site.

16. The information processing apparatus according to claim 1,
wherein the first processor is configured to
detect a treatment tool from the acquired image,
choose a plurality of treatment names corresponding to the detected treatment tool,
display the plurality of chosen treatment names in a third region on the screen of the first display unit,
accept selection of one treatment name from among the plurality of treatment names from start of the display until a third time elapses, and
stop acceptance of selection of the site while selection of the treatment name is accepted.

17. The information processing apparatus according to claim 16,
wherein the first processor records information on the selected treatment name in association with information on the selected site.

18. The information processing apparatus according to claim 1,
wherein the first processor is configured to
perform recognition processing on the acquired image, and
record a result of the recognition processing in association with information on the selected site.

19. The information processing apparatus according to claim 18,
wherein the first processor performs the recognition processing on the image captured as a static image.

20. The information processing apparatus according to claim 19,
wherein the first processor displays first information indicating the result of the recognition processing in a fourth region on the screen of the first display unit.

21. The information processing apparatus according to claim 20,
wherein the first processor is configured to
accept adoption or rejection of the result of the recognition processing indicated by the first information, and
record the result of the recognition processing in a case where the result of the recognition processing is adopted.

22. The information processing apparatus according to claim 21,
wherein the first processor accepts only a rejection instruction, and confirms adoption in a case where the rejection instruction is not accepted from start of the display of the first information until a fourth time elapses.

23. The information processing apparatus according to claim 18,
wherein the first processor is configured to
generate second information indicating a result of the recognition processing for each of the sites, and
display the second information on the first display unit.

24. The information processing apparatus according to claim 23,
wherein the first processor is configured to
divide a first site for which a plurality of the results of the recognition processing are recorded, among the plurality of sites, into a plurality of second sites, and
generate the second information indicating the result of the recognition processing for each of the second sites, regarding the first site.

25. The information processing apparatus according to claim 24,
wherein the first processor is configured to
set the second sites by equally dividing the first site, and
generate the second information by assigning the results of the recognition processing to the second sites in chronological order along an observation direction.

26. The information processing apparatus according to claim 23,
wherein the first processor generates the second information using a schema diagram.

27. The information processing apparatus according to claim 23,
wherein the first processor generates the second information using a strip graph divided into a plurality of regions.

28. The information processing apparatus according to claim 23,
wherein the first processor generates the second information indicating the result of the recognition processing using a color or a density.

29. The information processing apparatus according to claim 23,
wherein the first processor determines severity of ulcerative colitis using the recognition processing.

30. The information processing apparatus according to claim 29,
wherein the first processor determines the severity of the ulcerative colitis using Mayo Endoscopic Subscore.

31. The information processing apparatus according to claim 1,
wherein the first processor accepts selection of the site after insertion of the endoscope is detected or after insertion of the endoscope is confirmed by a user input.

32. The information processing apparatus according to claim 1,
wherein the first processor accepts selection of the site until pulling-out of the endoscope is detected or until pulling-out of the endoscope is confirmed by a user input.

33. The information processing apparatus according to claim 1,
wherein the first processor is configured to
detect a treatment tool from the acquired image,
display a plurality of options regarding a treatment target in a fifth region on the screen of the first display unit in a case where the treatment tool is detected from the image, and
accept selection of one of the plurality of options displayed in the fifth region.

34. The information processing apparatus according to claim 33,
wherein the plurality of options regarding the treatment target are a plurality of options for a detailed site or a size of the treatment target.

35. The information processing apparatus according to claim 1,
wherein the first processor is configured to
display a plurality of options regarding a region of interest in a fifth region on the screen of the first display unit in a case where a static image to be used in a report is acquired, and
accept selection of one of the plurality of options displayed in the fifth region.

36. The information processing apparatus according to claim 35,
wherein the plurality of options regarding the region of interest are a plurality of options for a detailed site or a size of the region of interest.

37. The information processing apparatus according to claim 1,
wherein the first processor records a captured static image in association with information on the selected site.

38. The information processing apparatus according to claim 37,
wherein the first processor records, as a candidate for an image to be used in a report or a diagnosis, the captured static image in association with the information on the selected site.

39. The information processing apparatus according to claim 38,
wherein the first processor acquires, as the candidate for the image to be used in the report or the diagnosis, a most recent static image in terms of time among static images captured before a time point when selection of the site is accepted, or an oldest static image in terms of time among static images captured after the time point when the selection of the site is accepted.

40. A report creation support device that supports creation of a report, comprising:
a second processor,
wherein the second processor is configured to
display a report creation screen with at least an input field for a site, on a second display unit,
acquire information on the site selected in the information processing apparatus according to claim 1,
automatically input the acquired information on the site to the input field for the site, and
accept correction of the automatically input information of the input field for the site.

41. The report creation support device according to claim 40,
wherein the second processor displays the input field for the site such that the input field for the site is distinguishable from other input fields on the report creation screen.

42. A report creation support device that supports creation of a report, comprising:
a second processor,
wherein the second processor is configured to
display a report creation screen with at least input fields for a site and a static image, on a second display unit,
acquire information on the site and the static image selected in the information processing apparatus according to claim 37,
automatically input the acquired information on the site to the input field for the site,
automatically input the acquired static image to the input field for the static image, and
accept correction of the automatically input information of the input field for the site and the automatically input static image of the input field for the static image.

43. An endoscope system comprising:
an endoscope;
the information processing apparatus according to claim 1; and
an input device.

44. An information processing method comprising:
a step of acquiring an image captured using an endoscope;
a step of displaying the acquired image in a first region on a screen of a first display unit;
a step of detecting a specific region in a hollow organ from the acquired image;
a step of displaying a plurality of sites constituting the hollow organ to which the detected specific region belongs, in a second region on the screen of the first display unit; and
a step of accepting selection of one site from among the plurality of sites.
